(19) United States
(12) Patent Application Publication
(10) Pub. No.: US 2010/0204058 A1
(43) Pub. Date: Aug. 12, 2010

(54) Profiling for Determination of Response to Treatment for Inflammatory Disease

(76) Inventors: Howard Yuan-Hao Chang, Stanford, CA (US); William H. Robinson, Palo Alto, CA (US); David Fiorentino, Stanford, CA (US); Lorinda Chung, Stanford, CA (US); Adam Adler, Emerald Hills, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/694,980

(22) Filed: Jan. 27, 2010

Related U.S. Application Data
(60) Provisional application No. 61/147,983, filed on Jan. 28, 2009.

(51) Int. Cl. C40B 30/04; C12Q 1/68; C40B 40/06; C07H 21/00

(52) U.S. Cl. 506/9; 435/6; 506/16; 536/24.33; 536/24.3

(57) ABSTRACT

The present invention relates to compositions and methods for treating, characterizing, and diagnosing autoimmune diseases or other inflammatory diseases. In particular, the present invention provides gene expression profiles as well as novel TKI Responsive Signature(s) useful for the diagnosis, characterization, prognosis and treatment of autoimmune disease or other inflammatory diseases.

This invention was made with support from the National Institutes of Health. The Government has certain rights in this invention.

### BACKGROUND OF THE INVENTION

Autoimmune diseases and other inflammatory diseases are estimated to affect 5% of the U.S. and world populations (Jacobson et al. (1997) Clin Immunol. Immunopathol. 84:223-43). In normal individuals immune responses provide protection against viral and bacterial infections. In autoimmune diseases and other inflammatory diseases, these same cellular responses involve host tissues, causing organ and/or tissue damage, e.g., to the joints, skin, pancreas, brain, thyroid, lungs, liver or gastrointestinal tract. Further manifestations of inflammatory disorders are caused by dysregulated host cell responses in the chronic inflammatory state. More than 100 distinct autoimmune diseases and other inflammatory diseases exist, and examples include rheumatoid arthritis, multiple sclerosis, Crohn's disease, psoriasis, primary biliary cirrhosis, systemic sclerosis, idiopathic pulmonary fibrosis and other diseases. The methods and compositions to be described relate to responsive signatures for the treatment of inflammatory disorders.

### Tyrosine Kinases

Phosphorylation of target proteins by kinases is an important mechanism in signal transduction and for regulating enzyme activity. Tyrosine kinases (TK) are a class of over 100 distinct enzymes that transfer a phosphate group from ATP to a tyrosine residue in a polypeptide (Table 1). Tyrosine kinases phosphorylate signaling, adaptor, enzyme and other polypeptides, causing such polypeptides to transmit signals to activate (or inactive) specific cellular functions and responses. There are two major subtypes of tyrosine kinases, receptor tyrosine kinases and cytoplasmic/non-receptor tyrosine kinases.

### Receptor Tyrosine Kinases

To date there have been approximately 60 receptor tyrosine kinases (RTKs; also known as tyrosine receptor kinases (TRK)) described in humans. These kinases are high affinity receptors for hormones, growth factors and cytokines (Robinson et al. (2001) Oncogene 19:5548-57). The binding of hormones, growth factors and/or cytokines generally activates these kinases to promote cell growth and division. Exemplary kinases include insulin-like growth factor receptor, epidermal growth factor receptor, platelet-derived growth factor receptor, etc. Most receptor tyrosine kinases are single subunit receptors but some, for example the insulin receptor, are multimeric complexes. Each monomer contains an extracellular N-terminal region, a single transmembrane spanning domain of 25-38 amino acids, and a C-terminal intracellular domain. The extracellular N-terminal region is composed of a very large protein domain which binds to extracellular ligands e.g. a particular growth factor or hormone. The C-terminal intracellular region provides the kinase activity of these receptors. To date, approximately 20 different subclasses of receptor tyrosine kinases have been identified (Robinson et al. (2001) Oncogene 19:5548-57). Receptor tyrosine kinases are key regulators of normal cellular processes and play a critical role in the development and progression of many types of cancer (Zwick et al. (2001) Endocr. Relat. Cancer 8:161-173).

RTKs include an extracellular binding site for their ligand, a transmembrane domain, and a kinase domain within the cytoplasm. The RTKs further include an ATP-binding site, a domain to bind the kinase substrate, and a catalytic site to transfer the phosphate group. The catalytic site lies within a cleft which can be in an open (active) or closed (inactive) form. The closed form allows the substrate and other residues to be brought into the catalytic site, and the open form grants access to ATP to drive the catalytic reaction (Roskoski, R. (2005) Biochem. Biophys. Res. Commun. 338:1307-15).

The class III RTKs, which include PDGFRa, PDGFRb, c-Fms, c-Kit and Fms-like tyrosine kinase 3 (Flt-3) (Table 1), are distinguished from other classes of RTKs in having five immunoglobulin-like domains within their extracellular binding site as well as a 70-100 amino acid insert within the kinase domain (Roskoski, R. (2005) Biochem. Biophys. Res. Commun. 338:1307-15). Structural similarities among class III RTKs results in cross-reactivity with respect to ligands, as evidenced in the case of imatinib blocking PDGFRa, PDGFRb, c-Fms, and c-Kit. Platelet-derived growth factor receptors (PDGFR) include PDGFR-alpha (PDGFRa) and the PDGFR-beta (PDGFRb) (Yu, J. et al, (2001) Biochem Biophys Res Commun. 282:697-700). The PDGF B-chain homodimer PDGF BB activates both PDGFRa and PDGFRb, and promotes proliferation, migration and other cellular functions in fibroblast, smooth muscle and other cells. The PDGF-A chain homodimer PDGF AA activates PDGFRa only. PDGF-AB binds PDGFRa with high-affinity and in the absence of PDGFRa can bind at a lower affinity (Seifert, R. A., et al, (1993), J Biol Chem. 268(6):4473-80). Recently, additional PDGFR ligands have been identified including PDGF-CC and PDGF-DD. Fibroblasts and other mesenchymal cells express fibroblast-growth factor receptor (FGFR) which mediates tissue repair, wound healing, angiogenesis and other cellular functions.

There are several direct and indirect ways to block tyrosine kinase activity, including: (i) competitive inhibition of ATP binding site, (ii) interfering with the cleft transition from open to closed forms (i.e., stabilizing either the open or closed forms), (iii) directly blocking the substrate from binding to the binding site of a tyrosine kinases, and (iv) blocking production or recruitment of ligand or substrate. Imatinib, CGP53716 and GW2580 are examples of small molecule tyrosine kinase inhibitors that are competitive inhibitors of ATP binding to the kinase. Imatinib binds the closed (inactive) form of Abl, while the open (active) form is sterically incompatible for imatinib binding. ATP cannot bind to the TK when imatinib is bound, and the substrate cannot be phosphorylated. The small molecule tyrosine kinase inhibitors approved to date bind the ATP-binding site and block ATP from binding, thereby inhibiting the tyrosine kinase from phosphorylating its substrate target. Table 1 provides a list of protein tyrosine kinases.

TABLE 1
Tyrosine Kinases (TKs): Overview of Cellular Distributions and Cellular
Functions
Tyrosine kinase Receptor:

| | |
|---|---|
| Cells expressing kinase | Cellular function |

PDGFR family (Class III RTKs):

| | | |
|---|---|---|
| c-Fms | Monocytes, macrophages, osteoclasts | Cell growth, proliferation, differentiation, |
| | | survival, and priming |
| PDGFRa | Fibroblasts, smooth muscle cells, | Cell growth, proliferation, differentiation and |
| | keratinocytes, glial cells, chondrocytes | survival |
| PDGFRb | Fibroblasts, smooth muscle cells, | Cell growth, proliferation, differentiation and |
| | keratinocytes, glial cells, chondrocytes | survival |
| c-Kit | Haematopoietic progenitor cells, mast | Cell growth, proliferation, differentiation and |
| | cells, primordial germ cells, interstitial | survival |
| | cells of Cajal | |
| Flt-3 | Haematopoietic progenitor cells | Cell growth, proliferation, differentiation and |
| | | survival |

VEGFR family:

| | | |
|---|---|---|
| VEGFR1 | Monocytes, macrophages, endothelial | Monocyte and macrophage migration; vascular |
| | cells | permeability |
| VEGFR2 | Endothelial cells | Vasculogenesis; angiogenesis |
| VEGFR3 | Lymphatic endothelial cells | Vasculogenesis; lymphangiogenesis |
| FGFR | Fibroblasts and other mesenchymal | Tissue repair, wound healing, angiogenesis |
| family: | cells | |

Non-receptor (cytoplasmic):

| | | |
|---|---|---|
| ABL family: | Ubiquitous | Cell proliferation, survival, cell adhesion and |
| | | migration |

JAK family:

| | | |
|---|---|---|
| JAK1 | Ubiquitous | Cytokine signaling |
| JAK2 | Ubiquitous | Hormone-like cytokine signaling |
| AK3 | T cells, B cells, NK cells, myeloid cells | common-gamma chain cytokine signaling |
| TYK2 | Ubiquitous | Cytokine signaling |

SRC-A family:

| | | |
|---|---|---|
| FGR | Myeloid cells (monocytes, | Terminal differentiation |
| | macrophages, granulocytes) | |
| FYN | Ubiquitous | Cell growth; T cell receptor, regulation of brain |
| | | function, and adhesion mediated signaling |
| SRC | Ubiquitous | Cell development, growth, replication, adhesion, |
| | | motility |
| YES | Ubiquitous | Maintaining tight junctions; transmigration of IgA |
| | | across epithelial cells |

SRC-B family:

| | | |
|---|---|---|
| BLK | B cells, thymocytes | B cell proliferation and differentiation; thymopoiesis |
| HCK | Myeloid cells, lymphoid cells | Proliferation, differentiation, migration |
| LCK | T cells, NK cells | T-cell activation; KIR activation |
| LYN | Myeloid cells, B cells, mast | BCR signaling; FceR1 signaling |
| | cells | |

SYK family:

| | | |
|---|---|---|
| SYK | Ubiquitous | Proliferation, differentiation, phagocytosis; tumor |
| | | suppressor |
| ZAP70 | T cells, NK cells | T-cell activation; KIR activation |

### SUMMARY OF THE INVENTION

The present invention relates to compositions and methods for treating, characterizing, and diagnosing autoimmune diseases and other inflammatory diseases. In particular, the present invention provides novel tyrosine kinase inhibitor responsive gene signatures (TKI Responsive Signature) useful for the diagnosis, characterization, and treatment of autoimmune diseases and other inflammatory diseases. The present invention further provides tyrosine kinase inhibitor responsive signatures that, when detected in a sample as a gene expression profile, act as significant predictors of clinical outcome.

The present invention relates to compositions and methods for characterizing and treating autoimmune diseases and other inflammatory diseases. In particular, the present invention provides TKI Responsive Signatures useful for the selection of treatment for autoimmune diseases and other inflammatory diseases. The TKI Responsive Signatures comprises the genes and polypeptides encoded by the genes that are differentially expressed in the selected autoimmune diseases and other inflammatory diseases, and an example is shown in Table 2.

In certain embodiments, the present invention provides methods of determining the presence or absence of a TKI Responsive Signature, comprising: a) providing a biological sample from a subject, and b) detecting gene or polypeptide expression in the biological sample under conditions such that the presence or absence of a TKI Responsive Signature in the tissue sample is determined. In certain embodiments, the methods of the present invention further comprise guiding selection of a particular therapeutic agent to treat the patient, for example a small molecule TKI.

In certain embodiments, detecting a TKI Responsive Signature comprises determining the expression levels of polynucleotides comprising a TKI Responsive Signature. In certain embodiments, the detecting of a TKI Responsive Signature profile comprises detecting mRNA expression comprising a TKI Responsive Signature. In some embodiments, the detection of mRNA expression is via Northern blot. In some embodiments, the detection of mRNA expression is via RT-PCR, real-time PCR or quantitative PCR using primer sets that specifically amplify the polynucleotides comprising the TKI Responsive Signature gene set. In certain embodiments, the detection of mRNA comprises exposing a sample to nucleic acid probes complementary to polynucleotides comprising a TKI Responsive Signature. In some embodiments, the mRNA of the sample is converted to cDNA prior to detection. In some embodiments, the detection of mRNA is via microarrays that comprise a TKI Responsive Signature. The number of genes in a TKI Responsive Signature is usually at least 3, 4, 5, 6, 7, 8, 9, at least 10, at least 20, at least 25, at least 30, at least 35, at least 40, at least 45, or the set of 49 genes, as set forth in Tables 2 and 3, herein.

In certain embodiments, the determining of expression levels of one or more genes in a biological sample of the patient afflicted with an autoimmune disease or other inflammatory disease comprises detecting polypeptides encoded by polynucleotides comprising a TKI Responsive Signature. In some embodiments, the detection of polypeptide expression comprises exposing a sample to antibodies specific to the polypeptides and detecting the binding of the antibodies to the polypeptides by, for example, quantitative immunofluorescence or ELISA. Other detection means are known to one of ordinary skill in the art see e.g., U.S. Pat. No. 6,057,105.

In certain embodiments, reagents and methods for predicting a subject's clinical outcome (including, but not limited to, disease progression and response to therapy with a TKI) are provided using the TKI Responsive Signature of the present invention. TKI Responsive Signature comprising identified genes involved in multiple functional pathways, including cell proliferation, matrix and vascular remodeling, immune signaling, immune function and growth factor signaling are provided that are predictive of disease progression and survival and can thus be used to classify patients afflicted with an autoimmune disease or other inflammatory disease into responsive or non-responsive subclasses and further provide a diagnosis, provide a prognosis, select a therapy, or monitor a therapy. In certain embodiments, a method of classifying an autoimmune disease or other inflammatory disease comprises: a) providing a patient sample, for example by obtaining a lesional biopsy from a subject; b) determining expression or activity of at least one polynucleotide or polypeptide selected from a TKI Responsive Signature; and c) classifying the patient with the autoimmune disease or other inflammatory disease as belonging to a TKI non-responsive class or a TKI responsive class based on the results of b). In certain embodiments, the method further comprises providing a diagnosis, prognosis, selecting a therapy, or monitoring a therapy.

According to certain of the inventive methods, the presence or amount of a gene product, e.g., a polypeptide or a nucleic acid is detected in a sample derived from a subject (e.g. a sample of tissue or cells obtained from a patient afflicted with an autoimmune or other inflammatory disease or a blood sample obtained from the subject). In certain embodiments, the subject is a human. In some embodiments, the subject is an individual who has or can have an autoimmune or other inflammatory disease. The sample can be subjected to a number of processing steps prior to or in the course of detection.

In some embodiments of the invention, hierarchical clustering can be used to assess the similarity between a TKI Responsive Signature and the TKI Responsive Signature gene expression profile from a patient sample. In other embodiments, a decision tree algorithm is used to identify patients with clinically meaningful differences in outcome. Other methods may utilize classification algorithms, regression analysis, principal components analysis, multivariate analysis, predictive models, and combinations thereof.

In another embodiment, prognostic algorithms are provided, which combine the results of multiple expression determinations and/or other clinical and laboratory parameters, and which will discriminate between individuals who will respond to the TKI therapy of interest, and those who will not respond. In some embodiments TKI Responsive Signature profiles are analyzed in combination with clinical, imaging, laboratory and genetic parameters to assess an individual patient's disease state and thereby determine if they would benefit from initiation of TKI therapy.

In one use of such an algorithm, a reference dataset is obtained, which comprises, as a minimum, a TKI Responsive Signature profile as identified herein. Such a database may include positive controls representative of disease subtypes; and may also include negative controls. The dataset optionally includes a profile for clinical indices, metabolic measures, genetic information, and the like. The disease dataset is then analyzed to determine statistically significant matches between datasets, usually between reference datasets and test datasets and control datasets. Comparisons may be made between two or more datasets.

In certain embodiments, the present invention provides kits for detecting autoimmune disease or other inflammatory disease expression profiles in a subject, comprising: a) at least one reagent capable of specifically detecting at least one gene of a subset of genes from the TKI Responsive Signature gene set in a biological sample, such as tissue or cell sample from a subject with an autoimmune disease or other inflammatory disease, and b) instructions for using the reagent(s) for detecting the presence or absence of an TKI Responsive Signature profile in the biological sample. In some embodiments, the at least one reagent comprises nucleic acid probes complementary to mRNA of at least one gene of a TKI Responsive Signature. In some embodiments, the at least one reagent comprises antibodies or antibody fragments that specifically bind to at least one gene product of a TKI Responsive Signature.

Examples of autoimmune diseases and other inflammatory diseases from which samples can be isolated or enriched for use in accordance with the invention include, but are not limited to, rheumatoid arthritis, multiple sclerosis, inflammatory bowel diseases (Crohn's disease, ulcerative colitis, and other inflammatory bowel diseases), systemic lupus ertythematosius (SLE), psoriasis, systemic sclerosis, autoimmune diabetes thyroid (Grave's disease and Hashimoto's thyroiditis), autoimmune diseases involving the peripheral nerves (Guillain-Barre Syndrome and other autoimmune peripheral neuropathies), autoimmune diseases involving the CNS (in addition to MS, acute disseminated encephalomyelitis [ADEM] and neuromyelitis optica [NMO]), autoimmune diseases involving the skin (in addition to psoriasis, pemphigoid (bullous), pemphigus foliaceus, pemphigus vulgaris, coeliac sprue-dermatitis, and vitiligo), the liver and gastrointestinal system (primary biliary cirrhosis, pernicious anemia, autoimmune hepatitis), the lungs (systemic sclerosis, pulmonary artery hypertensions, idiopathic pulmonary fibrosis) and the eye (autoimmune uveitis). There are also multiple “autoimmune rheumatic” autoimmune diseases and other inflammatory diseases including Sjögren's syndrome, discoid lupus, antiphospholipid syndrome, CREST, mixed connective tissue disease (MCTD), polymyositis and dermatomyositis, and Wegener's granulomatosus.

The present invention thus provides for the first time a TKI Responsive Signature that is predictive of clinical outcome in response to treatment with a TKI. The TKI Responsive Signatures shown in Tables 2, 3, 5, 6, 7, or 8 are established as predictive of a response to TKI therapy. In some embodiments of the present invention, the TKI Responsive Signature is used clinically to classify a patient afflicted with an autoimmune disease or other inflammatory disease as low-responsive or non-responsive to TKI treatment, or likely to be responsive or responsive to TKI treatment category. The TKI Responsive Signature can further be used to provide a diagnosis, prognosis, and select a therapy based on the classification of a patient with the particular autoimmune disease or other inflammatory disease as low-responsive or non-responsive to TKI treatment, or likely to be responsive, or responsive to TKI treatment as well as to monitor the response to therapy over time. In some embodiments, the TKI Responsive Signature can be used experimentally to test and assess lead compounds including, for example, small molecules, siRNAs, genetic therapies, and antibodies for the inhibition of tyrosine kinases to treat an autoimmune disease or other inflammatory disease.

Compositions and methods are provided for prognostic classification of individuals into groups that are informative of the individual's responsiveness to a therapy of interest. Therapies of interest include the administration of tyrosine kinase inhibitors. Examples of such inhibitors include imatinib, sorafenib, sunitinib, dasatinib, axitinib, nilotinib, pazopanib, batalanbib, cediranib, ZIRINIV, Rnsuriniv, AMG06, MLN518, AZD0530 and analogs or mimetics thereof. Autoimmune diseases and other inflammatory diseases of interest include, without limitation, autoimmune diseases and other inflammatory diseases such as systemic sclerosis, rheumatoid arthritis, Crohn's disease, graft-vs-host disease, primary biliary cirrhosis, pulmonary artery hypertension, psoriasis, multiple sclerosis, etc.

Other features, objects, and advantages of the invention will be apparent from the detailed description below.

### BRIEF DESCRIPTION OF THE DRAWINGS

FIG. 1. Effect of TKI (imatinib) on digital ulcers, interstitial lung disease, and collagen architecture in a patient with SSc. (A) Digital ulcer located over the left fourth proximal interphalangeal joint prior to imatinib therapy. (B) Healing of digital ulcer after 3 months of imatinib therpy. (C) HRCT of the chest prior to imatinib therapy demonstrates patchy infiltrates associated with ground glass opacities in the bilateral lower lobes. (D) HRCT after 3 months of imatinib therapy shows resolution of ground glass opacities. (E) Hematoxylin and eosin stained skin biopsy from the right arm taken prior to imatinib therapy shows dense, eosinophilic, tightly packed collagen bundles of the papillary and reticular dermis with an average dermal thickness of 2.81 mm (Magnification 100×). (F) Skin biopsy after 3 months of imatinib taken within 1 cm of initial biopsy shows normalization of collagen architecture, with loose spacing and thinning of collagen bundles and an average dermal thickness of 2.31 mm.

FIG. 2. Imatinib reduces PDGFRb and Abl activation in SSc skin and function in SSc fibroblasts. (A-D) Immunohistochemical staining of serial skin biopsy samples obtained pre-treatment (A,C) and one month following the initiation of imatinib treatment (B,D) with anti-phospho-PDGFRb (A,B) and anti-phospho-Abl (C,D) antibodies. Boxed areas of upper panels (200× magnification), are presented at higher magnification in their corresponding lower panels (600×). Results are representative of those obtained from multiple sections from two independent patients. Phospho-PDGFRb was observed in interstitial fibroblasts as well as perivascular spindle-like cells and some cells resembling mast cells. Phospho-Abl was observed in endothelial cells in small vessels and in scattered dermal fibroblasts. (E) Stimulation of a SSc fibroblast line with PDGF (10 ng/ml), TGF-b (0.5 ng/ml), PDGF+TGF-b, or PDGF+TGF-b+imatinib (1 mM). Proliferation was quantitated after 48 hours by 3H-thymidine incorporation (Y axis). Results are representative of experiments performed on two independent SSc fibroblast lines, and similar results were obtained with normal fibroblast lines.

FIG. 3. An TKI Responsive Signature is present in most diffuse SSc. (A) The TKI Responsive Signature was determined by applying Significance Analysis of Microarrays (SAM) to identify mRNA that exhibited statistically significant changes in their levels in pre-treatment as compared to post-treatment skin biopsy samples derived from the two TKI (imatinib)-treated SSc patients. SAM identified 1050 genes that were changed by imatinib therapy in both patients (FDR<0.001), and this TKI Responsive Signature is represented by the bar to the left of the heatmap image (red represents an increase, and green a decrease, in mRNA expression post-treatment; the genes comprising the TKI Responsive Signature are presented in Table 3. The genes comprising the TKI Responsive Signature were then used to organize via unsupervised hierarchical clustering the 75 gene expression profiles derived from skin biopsies from SSc, limited SSc/CREST, morphea and health control patients contained in a database. The results of the hierarchical clustering are presented as a heatmap, with each column representing the mRNA profile of a sample, and rows representing the genes present in the TKI Responsive Signature. Unsupervised hierarchical clustering revealed two distinct clusters, with the TKI Responsive Signature expression pattern being similar to one of the clusters, and this cluster being highly enriched for diffuse SSc samples (29 out of the 31 gene expression profiles contained in this cluster are from diffuse SSc, P<10-8, chi-square). This cluster of gene expression profiles derived from most of the diffuse SSc samples exhibited a pattern of gene activation and repression concordant with the TKI Responsive Signature, including alterations in the expression of genes involved in cell proliferation (red), immune signaling (blue), matrix remodeling (tan), and growth factor signaling (pink) (indicated to the right of the heatmap). The other cluster contained most of the profiles derived from limited/CREST, morphea and normal subjects, and the gene expression profiles from these patients did not exhibit the imatinib-responsive signature (this cluster contains 44 gene expression profiles, including 14 from normal skin, 15 from limited SSc/CREST, 5 from morphea, and 10 from diffuse SSc). (B) Reduction in the wound signature by imatinib in two patients with SSc. Replicate array analysis was performed for each sample; mean+standard deviation is shown.

FIG. 4. Cell types that contribute to the TKI Responsive Signature derived in FIG. 3. The TKI-responsive gene expression signature was isolated from gene expression profiles of 11 individual cell types that are likely to be present in skin. Using UniGene ID to convert the genes, 485 of 1050 imatinib-responsive genes were isolated. Imatinib-responsive genes that are specifically expressed in a given cell type are highlighted on the right. The percentages of the genes specifically expressed in fibroblasts, endothelial cells, B-cells, or multiple cell types are provided.

FIG. 5. A 49 gene TKI Responsive Signature is identified in multiple autoimmune diseases and other inflammatory diseases. (A) Seventy-five gene expression profiles derived from Scleroderma (SSc) skin biopsies (the disease subtype is indicated for each sample by color) were analyzed by unsupervised hierarchical clustering. The expression pattern of the 49 gene TKI Responsive Signature prior to TKI (Imatinib) treatment are represented by the bar on the left of the heatmap image (red indicates increased expression, green indicates decreased expression). (B) Fifteen gene expression profiles of Rheumatoid arthritis (RA) and Osteoarthritis (OA) synovial tissues were analyzed by unsupervised hierarchical clustering. (C) Thirty-six gene expression profiles of Crohn's disease (CD), Ulcerative colitis (UC), infectious colitis (INF), and Normal Control (Normal) bowel biopsies were analyzed by unsupervised hierarchical clustering. (D) Twenty-six gene expression profiles of lung biopsies derived from patients with Idiopathic pulmonary fibrosis were analyzed by unsupervised hierarchical clustering. This dataset included one gene profile from a patient with SSc, and one gene profile from a patient with mixed connective tissue disease (MCTD).

FIG. 6. Identification of core PDGFR-Abl-Kit and PDGFR-Abl-Kit-Fms TKI Responsive Signatures. (A) To identify a core set of genes that distinguish autoimmune diseases driven by the PDGFR, Abl, and Kit tyrosine kinases, gene expression profiles from samples derived from Scleroderma and Idiopathic pulmonary fibrosis patients were clustered with all 1050 TKI Responsive Signature genes. Genes that robustly distinguish the disease samples and normal controls were identified for each disease type, and the overlap between the two lists of genes formed a core PDGFR-Abl-Kit Responsive Signature comprising 22 genes. (B) Seventy-five gene expression profiles of Scleroderma samples (top) and 26 gene expression profiles of Fibrosis samples (bottom) were analyzed by unsupervised hierarchical clustering of the 22 genes comprising the PDGFR-Abl-Kit Responsive Signature. (C) To identify genes that distinguish autoimmune diseases driven by the PDGFR, Kit, and Fms tyrosine kinases, Crohn's disease/Ulcerative colitis and Rheumatoid arthritis/Osteoarthritis samples were clustered with all 1050 genes comprising the TKI Responsive Signature. Genes that robustly distinguish the disease samples and normal controls were identified for each disease type, and the overlap between the two lists of genes formed a core PDGFR-Kit-Fms Responsive Signature comprising 17 genes. (D) Nineteen gene expression profiles of Crohn's disease and Ulcerative colitis samples (top) and 15 gene expression profiles of Rheumatoid arthritis/Osteoarthritis samples (bottom) were analyzed by unsupervised hierarchical clustering of the 17 gene PDGFR-Kit-Fms Responsive Signature.

### DETAILED DESCRIPTION OF THE EMBODIMENTS

### Definitions

To facilitate an understanding of the present invention, a number of terms and phrases are defined below:

The terms “TKI Responsive Signature”, “TKI Gene Signature”, “TKI Responsive Gene Signature”, and grammatical equivalents are used interchangeably herein to refer to gene signatures comprising genes differentially expressed in response to the presence of a TKI in cells associated with an autoimmune disease or other inflammatory disease compared to those cells or population of cells or those cells in the absence of the TKI. In some embodiments, the TKI Responsive Signature comprises genes differentially expressed in selected cells associated with the autoimmune or other inflammatory disease versus stimulated cells in the absence of a TKI by a fold change, for example by 2-fold reduced and/or elevated expression, and further limited by using a statistical analysis, for example, statistical algorithms including hierarchical clustering, Significance Analysis of Microarrays (SAM; Tusher et al, Proc Natl Acad Sci USA. 2001 98(9):5116-21), Prediction Analysis of Microarrays (PAM; Tibshirani et al, Proc Natl Acad Sci USA. 2002 99(10):6567-72), or other algorithms. In some embodiments, the genes differentially expressed in response to the presence of a TKI in cells associated with the selected autoimmune or other inflammatory disease cells can be predictive both retrospectively and prospectively of responsiveness to selected TKI therapy for a particular autoimmune disease or other inflammatory disease.

“PDGFR, Abl, Kit, and Fms autoimmune disease or other inflammatory disease” refers to an autoimmune disease(s) and other inflammatory disease(s) that is in part mediated by dysregulated cellular responses regulated by the TKs PDGFR, Abl, Kit, and Fms. Examples of such cellular responses include PDGFR and Abl mediated fibroblast-lineage activation, proliferation and production of extracellular matrix, inflammatory mediator, and other products. Abl mediated activation of B cells produces autoantibodies. Kit-mediated mast cell activation produces and releases inflammatory mediators including bradykinin, histamine, cytokines, chemokines, and enzyme products. Fms-mediated differentiation of monocytes into macrophages and activation of macrophages produces inflammatory cytokines. The sequence of events resulting from alterations in cell proliferation, immune signaling, matrix remodeling, and growth factor signaling mediated by the PDGFR, Abl, Kit, and Fms TKs are characteristic of PDGFR, Abl, Kit, and Fms autoimmune diseases and other inflammatory diseases such as rheumatoid arthritis, multiple sclerosis, Crohn's disease, or psoriasis.

A “PDGFR, Abl, Kit, and Fms Responsive Signature” is a gene signature that arises due to and reflects excessive activation of PDGFR, Abl, Kit, and Fms with the consequent alterations in the expression of genes involved in PDGFR, Abl, Kit, and Fms mediated cell proliferation, immune signaling, matrix remodeling, and growth factor signaling.

“PDGFR, Kit, and Abl autoimmune disease or other inflammatory disease” refers to an autoimmune disease(s) and other inflammatory disease(s) that is in part mediated by dysregulated cellular responses regulated by the TKs PDGFR, Kit, and Abl. Examples of such cellular responses include PDGFR-mediate fibroblast-linage activation, proliferation and production of extracellular matrix, inflammatory mediator, and other products. Kit-mediated mast cell activation produces and releases inflammatory mediators including bradykinin, histamine, cytokines, chemokines, and enzyme products. Abl mediates activation of fibroblast-lineage cells, B-lineage cells, and other cell types. The sequence of events resulting from alterations in cell proliferation, immune signaling, matrix remodeling, and growth factor signaling mediated by the PDGFR, Kit, and Abl TKs are characteristic of PDGFR, Kit, and Abl autoimmune diseases and other inflammatory diseases such as systemic lupus erythrematosus, autoimmune hepatitis, primary biliary cirrhosis, idiopathic pulmonary fibrosis, or systemic sclerosis.

A “PDGFR, Kit, and Abl Responsive Signature” is a gene signature that arises due to and reflects excessive activation of PDGFR, Kit, and Abl with the consequent alterations in the expression of genes involved in PDGFR, Kit, and Abl cell proliferation, immune signaling, matrix remodeling, and growth factor signaling.

The term “class III tyrosine kinase receptors” refers to a subclass of receptor tyrosine kinases (RTKs). The class III RTKs, which include PDGFRa, PDGFRb, c-Fms, c-Kit and Fms-like tyrosine kinase 3 (Flt-3), are distinguished from other classes of RTKs in having five immunoglobulin-like domains within their extracellular binding site as well as a 70-100 amino acid insert within the kinase domain (Roskoski, R. (2005) Biochem. Biophys. Res. Commun. 338:1307-15). Structural similarities among class III RTKs results in cross-reactivity with respect to ligands, as evidenced in the case of imatinib blocking PDGFRa, PDGFRb, c-Fms, and c-Kit.

Platelet-derived growth factor receptors (PDGFR) include PDGFR-alpha (PDGFRa) and the PDGFR-beta (PDGFRb) (Yu, J. et al, (2001)Biochem Biophys Res Commun. 282:697-700). The PDGF B-chain homodimer PDGF BB activates both PDGFRa and PDGFRb, and promotes proliferation, migration and other cellular functions in fibroblast, smooth muscle and other cells. The PDGF-A chain homodimer PDGF AA activates PDGFRa only. PDGF-AB binds PDGFRa with high-affinity and in the absence of PDGFRa can bind at a lower affinity (Seifert, R. A., et al, (1993), J Biol Chem. 268(6):4473-80). Recently, additional PDGFR ligands have been identified including PDGF-CC and PDGF-DD. Fibroblasts and other mesenchymal cells express fibroblast-growth factor receptor (FGFR) which mediates tissue repair, wound healing, angiogenesis and other cellular functions.

As used herein, the terms “low levels”, “decreased levels”, “low expression”, “reduced expression” or “decreased expression” in regards to gene expression are used herein interchangeably to refer to expression of a gene or genes in a cell, population of cells or tissue, particularly a cell, population of cells, or tissue associated with the autoimmune disease and other inflammatory disease, at levels less than the expression of that gene in a second cell, population of cells or tissue, for example normal fibroblasts or normal skin. “Low levels” of gene expression refers to expression of a gene or genes in a cell, population of cells or tissue, particularly a cell, population of cells or tissue associated with the autoimmune disease and other inflammatory disease, at levels: 1) half that or below expression levels of the same gene in normal or control cells or 2) at the lower limit of detection using conventional techniques. “Low levels” of gene expression can be determined by detecting decreased to nearly undetectable amounts of a polynucleotide (mRNA, cDNA, etc.) in selected cells or tissue compared to control cells or tissue by, for example, quantitative RT-PCR or microarray analysis. Alternatively “low levels” of gene expression can be determined by detecting decreased to nearly undetectable amounts of the encoded protein or proteins in cells or tissue compared to control cells or tissue by, for example, ELISA, Western blot, quantitative immunofluorescence, protein array analysis, etc.

Flt3 is expressed in hematopoietic cells and is a class III receptor tyrosine kinase that also contributes to aberrant cellular responses in autoimmune and other inflammatory diseases. Flt3 activates a transcriptional program, upregulating specific genes and downregulating other specific genes, that contributes to TKI responsive gene signatures.

The terms “high levels”, “increased levels”, “high expression”, “increased expression” or “elevated levels” in regards to gene expression are used herein interchangeably to refer to expression of a gene or genes in a cell, population of cells or tissue, particularly a cell, population of cells or tissue associated with the autoimmune disease or other inflammatory disease, at levels higher than the expression of that gene or genes in a second cell, population of cells or tissue, particularly a cell, population of cells or tissue associated with the autoimmune disease or other inflammatory disease. “Elevated levels” of gene expression refers to expression of a gene in a cell, population of cells or tissue at levels twice that or more of expression levels of the same gene or genes in control cells or tissue. “Elevated levels” of gene expression can be determined by detecting increased amounts of a polynucleotide (mRNA, cDNA, etc.) in cells or tissue associated with an autoimmune disease or other inflammatory disease compared to control cells or tissue by, for example, quantitative RT-PCR or microarray analysis. Alternatively “elevated levels” of gene expression can be determined by detecting increased amounts of encoded protein in cells or tissue compared to control cells or tissue by, for example, ELISA, Western blot, quantitative immunofluorescence, etc.

The term “undetectable levels” or “loss of expression” in regards to gene expression as used herein refers to expression of a gene in a cell, population of cells or tissue, particularly a cell, population of cells or tissue associated with the autoimmune disease or other inflammatory disease, at levels that cannot be distinguished from background using conventional techniques such that no expression is identified. “Undetectable levels” of gene expression can be determined by the inability to detect levels of a polynucleotide (mRNA, cDNA, etc.) in cells or tissue above background by, for example, quantitative RT-PCR or microarray analysis. Alternatively “undetectable levels” of gene expression can be determined by the inability to detect levels of a protein in cells or tissue above background by, for example, ELISA, Western blot, immunofluorescence, etc.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having an autoimmune disease or other inflammatory disease” refers to a subject that presents one or more symptoms indicative of an autoimmune disease or other inflammatory disease who is being screened for an autoimmune disease or other inflammatory disease (e.g., during a routine physical). A subject suspected of having an autoimmune disease or other inflammatory disease can also have one or more risk factors. A subject suspected of having an autoimmune disease or other inflammatory disease has generally not been tested for an autoimmune disease or other inflammatory disease. However, a “subject suspected of having an autoimmune or other inflammatory disease” encompasses an individual who has received an initial diagnosis but for whom the severity of the disease is not known. The term further includes people who once had an autoimmune disease or other inflammatory disease (e.g., an individual in remission).

As used herein, the term “subject at risk for an autoimmune or other inflammatory disease” refers to a subject with one or more risk factors for developing an autoimmune or other inflammatory disease. Risk factors include, but are not limited to, gender, age, genetic predisposition, positive laboratory tests, environmental exposure, smoking cigarettes, previous incidents of an autoimmune disease or other inflammatory disease, family history of autoimmune diseases and other inflammatory diseases, and lifestyle.

As used herein, the term “subject diagnosed with an autoimmune disease or other inflammatory disease” refers to a subject(s) who have been examined, tested and found to have autoimmune or other inflammatory disease based on established diagnostic criteria. Established diagnostic criteria typically include one or more of the following: clinical symptoms (for example, joint pain, weakness in an limb, diarrhea, difficulty breathing, etc), findings on physical examination (for example, synovitis, motor weakness, abdominal tenderness, pulmonary crackles), laboratory test results (for example, blood rheumatoid factor, spinal fluid oligoclonal bands, etc), results from imaging studies (for example, bone erosions on hand X-rays, white matter lesions on brain magnetic resonance imaging, ground glass opacities on chest CT), results from invasive procedures and biopsies (for example, ulcerated mucosa on endoscopic examination, inflammatory cells in synovial fluid, specific features on histologic or molecular analysis of biopsy tissue), and the results from molecular studies including the ones described herein.

As used herein, the term “characterizing autoimmune or other inflammatory disease in a subject” refers to the identification of one or more properties of an autoimmune or other inflammatory disease sample in a subject, including but not limited to, clinical characteristics, laboratory characteristics, genetic characteristics, gene expression characteristics and protein expression characteristics. Clinical characteristics include, for example, symptoms and findings on physical examination reflective of conditions involving the skin, joints, lungs, liver, bowel, nervous system and other organs. An autoimmune or inflammatory disease can be characterized by the identification of the expression of one or more genes, including but not limited to, the genes/markers disclosed herein. Likewise, autoimmune disease or other inflammatory disease can be characterized by the identification of the expression and/or activation of one or more proteins, including but not limited to, the proteins disclosed herein.

As used herein, the terms “autoimmune or other inflammatory disease marker(s)”, refers to a gene or genes or a protein, polypeptide, or peptide expressed by the gene or genes whose expression level, alone or in combination with other genes, is correlated with the TKI Responsive Signature. The correlation can relate to either an increased or decreased expression of the gene (e.g. increased or decreased levels of mRNA, or the polypeptide or peptide encoded by the gene).

A “gene profile,” “gene pattern,” “expression pattern,” “expression profile,” “gene expression profile” or grammatical equivalents refer to identified expression levels of at least one polynucleotide or protein expressed in a biological sample and thus refer to a specific pattern of gene expression that provides a unique identifier of a biological sample, for example, an autoimmune disease or other inflammatory disease pattern of gene expression obtained by analyzing an autoimmune disease or other inflammatory disease sample in comparison to a reference sample will be referred to as a “TKI Responsive Signature gene profile” or a “TKI Responsive Signature expression pattern”. “Gene patterns” can be used to diagnose a disease, make a prognosis, select a therapy, and/or monitor a disease or therapy after comparing the gene pattern to a TKI Responsive Signature.

Correlation of gene signatures derived from a patient or group of patients with a particular disease, with the TKI Responsive Signature, can be determined using statistical methods and algorithms. An analytic classification process may use any one of a variety of statistical analytic methods to assess the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc. Using any one of these methods, a gene (or protein) expression dataset is used to generate a predictive signature profile.

The predictive TKI Responsive Signatures demonstrated herein utilize the results of multiple gene expression determinations, and provide an algorithm that will classify with a desired degree of accuracy an individual as belonging to a particular state, where a state may be autoimmune, inflammatory, or non-autoimmune or non-inflammatory. Classification of interest include, without limitation, the assignment of a sample to one or more of the autoimmune or other inflammatory disease states: (i) TKI responsive state versus TKI non-responsive state, (ii) PDGFR-Kit-Fms TKI responsive state versus PDGFR-Kit-Fms TKI non-responsive state, (iii) PDGFR-Kit-Abl TKI responsive state versus PDGFR-Kit-Abl TKI non-responsive state, (iv) small molecule therapeutic responsive state versus small molecule therapeutic non-responsive state, (v) biological therapeutic responsive state versus biological therapeutic non-responsive state, or (vi) need for additional tests versus no need for additional tests.

Classification can be made according to predictive methods that set a threshold for determining the probability that a sample belongs to a given class, such as a TKI responsive state. The probability preferably is at least 50%, or at least 60% or at least 70% or at least 80% or higher. Classifications also may be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In the development of a predictive signature, it may be desirable to select a subset of markers, i.e. at least 2, at least 3, at least 4, at least 5, at least 6, at least 7, at least 8, at least 9, at least 10, at least 15, at least 20, at least 25, at least 30, at least 40, at least 50 up to the complete set of markers. Usually a subset of markers will be chosen that provides for the needs of the quantitative sample analysis, e.g. availability of reagents, convenience of quantitation, etc., while maintaining a highly accurate predictive signature.

Also provided are reagents and kits thereof for practicing one or more of the above-described methods. The subject reagents and kits thereof may vary greatly. Reagents of interest include reagents specifically designed to analyze gene expression associated with autoimmune diseases or other inflammatory diseases, and response to TKIs. The kits may further include a software package for statistical analysis of one or more phenotypes, and may include a reference database for calculating the probability of classification. The kit may include reagents employed in the various methods, such as devices for withdrawing and handling blood samples, tubes, spin columns, DNA arrays and reagents, qPCR primers and reagents, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means may be present in the kits.

As used herein, the term “a reagent that specifically detects expression levels” refers to reagents used to detect the expression of one or more genes (e.g., including but not limited to, the autoimmune or other inflammatory disease responsive markers of the present invention). Examples of suitable reagents include but are not limited to, nucleic acid probes capable of specifically hybridizing to the gene of interest, PCR primers capable of specifically amplifying the gene of interest, and antibodies capable of specifically binding to proteins expressed by the gene of interest. Other non-limiting examples can be found in the description and examples below.

As used herein, the term “detecting a decreased or increased expression relative to control” refers to measuring the level of expression of a gene (e.g., the level of mRNA or protein) relative to the level in a control sample. Gene expression can be measured using any suitable method, including but not limited to, those described herein.

As used herein, the term “detecting a change in gene expression in a cell sample in the presence of said test compound relative to the absence of said test compound” refers to measuring an altered level of expression (e.g., increased or decreased) in the presence of a test compound relative to the absence of the test compound. Gene expression can be measured using any suitable method.

As used herein, the term “DNA arrays” includes microarrays used to perform multiplex characterization of mRNA expression. Such arrays are arrays of nucleic acids, or related molecules, that are used to hybridize to and thereby measure the levels of a many distinct mRNA simultaneously. Examples of DNA arrays include the Affymetrix HU-133 Plus 2.0 DNA array, the Agilent Whole Human Genome Oligo Microarray, the Stanford Functional Genomics Facility's HEEBO (human exon evidence-based oligonucleotide) arrays, as well as arrays produce by a variety of other sources.

As used herein, the term “instructions for using said kit for detecting an autoimmune disease or other inflammatory disease in said subject” includes instructions for using the reagents contained in the kit for the detection and characterization of an autoimmune disease or other inflammatory disease in a sample from a subject.

As used herein, “providing a diagnosis” or “diagnostic information” refers to any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a autoimmune disease or other inflammatory disease), information related to the nature or classification of a autoimmune disease or other inflammatory disease, information related to prognosis and/or information useful in selecting an appropriate treatment. Selection of treatment can include the choice of a particular tyrosine kinase inhibitor or other treatment modality, or a choice about whether to withhold or deliver therapy, etc.

As used herein, the terms “providing a prognosis”, “prognostic information”, or “predictive information” refer to providing information regarding the impact of the presence of an autoimmune disease or other inflammatory disease (e.g., as determined by the diagnostic methods of the present invention) on a subject's likelihood of responding to therapy, including tyrosine kinase inhibitor therapies, and future health (e.g., disease progression and death).

The term “responsive” in regards to those patients diagnosed with an autoimmune or other inflammatory disease who are likely to respond or have a higher probability of responding to TKI treatment as gene expression in their sample correlates with the TKI Responsive Signature than a patient having the autoimmune or other inflammatory disease whose gene expression in their samples did not correlate with the TKI Responsive Signature.

The term “non-responsive” in regards to patient(s) diagnosed with an autoimmune or other inflammatory disease or patient(s) who are unlikely to respond or have a lower probability of responding to TKI treatment as gene expression in their sample does not correlate than a patient with the autoimmune diseases or other inflammatory diseases whose gene expression profile does correlate with the TKI Responsive Signature. Correlation of gene signatures derived from a patient or group of patients with a particular autoimmune or other inflammatory disease, with the TKI Responsive Signature, is determined by statistical methods and algorithms as described above.

As used herein, the terms “biological sample”, “biopsy tissue”, “patient sample”, “autoimmune or other inflammatory disease sample” refers to a sample of cells, tissue or fluid that is removed from a subject for the purpose of determining if the sample contains autoimmune or other inflammatory disease tissue, for determining gene expression profile of that autoimmune disease or inflammatory disease tissue, or for determining the protein expression profile of that autoimmune or other inflammatory disease. In some embodiments, biopsy tissue or fluid is obtained because a subject is suspected of having an autoimmune or other inflammatory disease. The biopsy tissue or fluid is then examined for the presence or absence of autoimmune or inflammatory disease findings and/or TKI Responsive Signature expression. The biological sample, biological tissue, disease tissue or autoimmune disease tissue is obtained from autoimmune or other inflammatory disease tissue (e.g., blood samples, biopsy tissue) that has been removed from a subject (e.g., during phleobotomy or biopsies) and for example, may be a skin biopsy sample from a scleroderma patient; synovial tissue from an arthritis patient; intestinal biopsy sample from a Crohn's disease patient; lung biopsy in an idiopathic pulmonary fibrosis (IPF) patient, etc.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (e.g., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region can be present in a cDNA, genomic DNA or RNA form. When present in a DNA form, the oligonucleotide or polynucleotide can be single-stranded (i.e., the sense strand) or double-stranded. Suitable control elements such as enhancers/promoters, splice junctions, polyadenylation signals, etc. can be placed in close proximity to the coding region of the gene if needed to permit proper initiation of transcription and/or correct processing of the primary RNA transcript. Alternatively, the coding region utilized in the expression vectors of the present invention can contain endogenous enhancers/promoters, splice junctions, intervening sequences, polyadenylation signals, etc. or a combination of both endogenous and exogenous control elements.

As used herein the term “portion” when in reference to a nucleotide sequence (as in “a portion of a given nucleotide sequence”) refers to fragments of that sequence. The fragments can range in size from four nucleotides to the entire nucleotide sequence minus one nucleotide (10 nucleotides, 20, 30, 40, 50, 100, 200, etc.).

The phrases “hybridizes”, “selectively hybridizes”, or “specifically hybridizes” refer to the binding or duplexing of a molecule only to a particular nucleotide sequence under stringent hybridization conditions when that sequence is present in a complex mixture (e.g., a library of DNAs or RNAs). See, e.g., Andersen (1998) Nucleic Acid Hybridization Springer-Verlag; Ross (ed. 1997) Nucleic Acid Hybridization Wiley.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide. For high stringency hybridization, a positive signal is at least two times or 10 times background hybridization. Exemplary high stringency or stringent hybridization conditions include: 50% formamide, 5×SSC, and 1% SDS incubated at 42° C. or 5×SSC and 1% SDS incubated at 65° C., with a wash in 0.2×SSC and 0.1% SDS at 65° C. For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 sec, an annealing phase lasting 30-120 sec., and an extension phase of about 72° C. for 1-2 min.

Two-color labeling of nucleic acids derived from samples can be utilized in binding to the same or to separate arrays, in order to assay the level of binding in a sample compared to a control sample. From the ratio of one color to the other, for any particular array element, the relative abundance of ligands with a particular specificity in the two samples can be determined. In addition, comparison of the binding of the two samples provides an internal control for the assay. Competitive assays are well known in the art, where a competing samples of known specificity, may be included in the binding reaction.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide can be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide can be single-stranded), but can contain both the sense and anti-sense strands (i.e., the oligonucleotide or polynucleotide can be double-stranded).

As used herein the term “portion” when in reference to a protein (as in “a portion of a given protein”) refers to fragments of that protein. The fragments can range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA can be cleaved with restriction enzymes prior to electrophoresis. Following electrophoresis, the DNA can be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “RNA expression analysis,” as used herein refers to multiplex analysis of RNA by one of a variety of approaches. Examples of such approaches include DNA microarrays generated by printing oligonucleotides or in situ synthesis of oligonucleotides that will hybridize to the RNA produced from specific genes. RNA expression analysis can also be performed by multiplex PCR, where oligonucleotide primers are used to sequentially amplify nucleic acids sequences in RA derived from specific genes.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies can be detected by various methods, including the use of radiolabeled antibodies.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment. Mammalian species typically used for in vivo analysis include canines; felines; equines; bovines; ovines; etc. and primates, particularly humans. In vivo models, particularly small mammals, e.g. murine, lagomorpha, etc. may be used for experimental investigations. Animal models of interest include those for models of autoimmune diseases or other inflammatory diseases.

The terms “test compound” and “candidate compound” refers to any chemical entity, pharmaceutical, drug, and the like that is a candidate for use to treat or prevent a disease, illness, sickness, or disorder of bodily function (e.g., autoimmune disease or other inflammatory disease). Test compounds comprise both known and potential therapeutic compounds. A test compound can be determined to be therapeutic by screening using the screening methods of the present invention. In some

As used herein, the term “sample” includes a specimen or culture obtained from any source, as well as biological and environmental samples. Biological samples can be obtained from animals (including humans) and encompass fluids, solids, tissues, and gases. Biological samples include blood products, such as plasma, serum, as well as spinal fluid, joint fluid, and the like. In addition, biological samples include tissue obtained from tissue biopsies or the skin, lung, liver, colon, synovium, brain, muscle and other organs. Such examples are not however to be construed as limiting the sample types applicable to the present invention.

Before the subject invention is described further, it is to be understood that the invention is not limited to the particular embodiments of the invention described, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

Methods are also provided for optimizing therapy, by first classification, and based on that information, selecting the appropriate therapy, dose, treatment modality, etc. which optimizes the differential between delivery of an anti-proliferative treatment to the undesirable target cells, while minimizing undesirable toxicity. The treatment is optimized by selection for a treatment that minimizes undesirable toxicity, while providing for effective anti-proliferative activity.

Autoimmune Disease Or Other Inflammatory Disease. The compositions and methods of the invention find use in combination with a variety of autoimmune disease or other inflammatory conditions, which include, without limitation, the following conditions.

Fibrosis. Fibrosis is the formation or development of excess fibrous connective tissue in an organ or tissue as a reparative or reactive process, as opposed to formation of fibrous tissue as a normal constituent of an organ or tissue. Many autoimmune diseases or other inflammatory diseases result in the fibrosis of the targeted organ, which results in dysfunction. Inflammation resolution and fibrosis are inter-related conditions with many overlapping mechanisms, where macrophages, T helper cells, and fibroblasts each play important roles in regulating both processes. Following tissue injury, an inflammatory stimulus is often necessary to initiate tissue repair, where cytokines released from resident and infiltrating leukocytes stimulate proliferation and activation of fibroblasts. However, in many cases this drive stimulates an inappropriate pro-fibrotic response. In addition, activated fibroblasts can take on the role of traditional APCs, secrete pro-inflammatory cytokines, and recruit inflammatory cells to fibrotic foci, amplifying the fibrotic response in a vicious cycle.

Among the many pathologic conditions associated with fibrosis are included pulmonary fibrosis, renal fibrosis, hepatic fibrosis, cardiac fibrosis, and systemic sclerosis. Fibrotic processes in epithelial tissues (i.e. lung, liver, kidney and skin) share many of the same mechanisms and features, particularly epithelial-fibroblast cross-talk.

Systemic sclerosis is a rare chronic disease of unknown cause characterized by diffuse fibrosis, degenerative changes, and vascular abnormalities in the skin, joints, and internal organs (especially the esophagus, lower GI tract, lung, heart, and kidney). Common symptoms include Raynaud's syndrome, polyarthralgia, dysphagia, heartburn, and swelling and eventually skin tightening and contractures of the fingers. Lung, heart, and kidney involvement accounts for most deaths. Diagnosis is clinical, but laboratory tests help with confirmation. Emphasis is often on treatment of complications. Pathophysiology may involve vascular damage and activation of fibroblasts; collagen and other extracellular proteins in various tissues are overproduced.

Immunologic mechanisms and heredity (certain HLA subtypes) play a role in etiology. SSc-like syndromes can result from exposure to vinyl chloride, bleomycin, pentazocine, epoxy and aromatic hydrocarbons, contaminated rapeseed oil, or I-tryptophan.

In SSc, the skin develops more compact collagen fibers in the reticular dermis, epidermal thinning, loss of rete pegs, and atrophy of dermal appendages. T lymphocytes may accumulate, and extensive fibrosis in the dermal and subcutaneous layers develops. In the nail folds, capillary loops dilate and some microvascular loops are lost. In the extremities, chronic inflammation and fibrosis of the synovial membrane and surfaces and periarticular soft tissues occur.

SSc varies in severity and progression, ranging from generalized skin thickening with rapidly progressive and often fatal visceral involvement (SSc with diffuse scleroderma) to isolated skin involvement (often just the fingers and face) and slow progression (often several decades) before visceral disease develops. The latter form is termed limited cutaneous scleroderma or CREST syndrome (Calcinosis cutis, Raynaud's syndrome, Esophageal dysmotility, Sclerodactyly, Telangiectasias). In addition, SSc can overlap with other inflammatory rheumatic disorders, e.g., sclerodermatomyositis (tight skin and muscle weakness indistinguishable from polymyositis) and mixed connective tissue disease.

SSc may be classified as diffuse cutaneous (dcSSc) or limited cutaneous SSc (IcSSc). The latter is more insidious by nature, is associated with anticentromere antibodies, and is more vascular than is the more fibrotic diffuse form. Features of the CREST syndrome (calcinosis, Raynaud's phenomenon, esophageal dysmotility, sclerodactyly, telangiectasias) occur in both forms, but they differ in the extent of skin involvement. By nailfold capillaroscopy it has been shown that capillaries are both abnormal and reduced in number in both forms; neointima formation and media thickening also occur in both forms.

The immunologic abnormalities of SSc involve T and B lymphocytes. Early skin lesions show lymphocyte infiltration with enrichment of Th2 cells. Polarization of lymphocytes is also observed in the lungs of patients with dcSSc. Autoantibodies recognizing nuclear components are found in a majority of, if not all, patients with SSc and may define clinical subgroups. Autoantibodies are present early in the course of the disease, sometimes before the full-blown form develops, but they have not been shown to be directly pathogenic.

The search for effective antifibrotic agents in SSc has been a source of continuing disappointment. For many years D-penicillamine was the recommended antifibrotic therapy, but the first controlled trial showed no effect. More recently it was hoped that relaxin might be an effective antifibrotic therapy in SSc. Recent studies of cyclophosphamide indicate that this agent exerts significant but modest effects, confirming the findings of a number of earlier open-label trials, although long-term toxicity remains a problem with cyclophosphamide. Mycophenolate mofetil is used in several centers as an alternative to cyclophosphamide and seems to be well tolerated. However, no controlled data in support of its use are available. Immunoablation followed by autologous hematologic stem cell transplantation is at present under investigation in 2 controlled studies in progress in Europe and in the US. There are claims that the initial high-dose cyclophosphamide used for conditioning may be as effective as the complete stem cell treatment.

Distler et al. (2007) Arthritis Rheum. 56(1):311-22 investigated the effect of imatinib mesylate in SSc patients. Other studies have been published by Venalis et al. (2008) J Cell Mol Med.; Kay and High (2008) Arthritis Rheum. 58(8):2543-8; Pannu et al. (2008) Arthritis Rheum. 58(8):2528-37; and Soria et al. (2008) Dermatology. 216(2):109-17.

Rheumatoid Arthritis is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Environmental factors may also play a role. Immunologic changes may be initiated by multiple factors. About 0.6% of all populations are affected, women two to three times more often than men. Onset may be at any age, most often between 25 and 50 yr.

Prominent immunologic abnormalities that may be important in pathogenesis include immune complexes found in joint fluid cells and in vasculitis. Plasma cells produce antibodies that contribute to these complexes. Lymphocytes that infiltrate the synovial tissue are primarily T helper cells, which can produce pro-inflammatory cytokines. Macrophages and their cytokines (e.g., tumor necrosis factor, granulocyte-macrophage colony-stimulating factor) are also abundant in diseased synovium. Increased adhesion molecules contribute to inflammatory cell emigration and retention in the synovial tissue. Increased macrophage-derived lining cells are prominent along with some lymphocytes and vascular changes in early disease.

In chronically affected joints, the normally delicate synovium develops many villous folds and thickens because of increased numbers and size of synovial lining cells and colonization by lymphocytes and plasma cells. The lining cells produce various materials, including collagenase and stromelysin, which can contribute to cartilage destruction; interleukin-1, which stimulates lymphocyte proliferation; and prostaglandins. The infiltrating cells, initially perivenular but later forming lymphoid follicles with germinal centers, synthesize interleukin-2, other cytokines, RF, and other immunoglobulins. Fibrin deposition, fibrosis, and necrosis also are present. Hyperplastic synovial tissue (pannus) may erode cartilage, subchondral bone, articular capsule, and ligaments. PMNs are not prominent in the synovium but often predominate in the synovial fluid.

Onset is usually insidious, with progressive joint involvement, but may be abrupt, with simultaneous inflammation in multiple joints. Tenderness in nearly all inflamed joints is the most sensitive physical finding. Synovial thickening, the most specific physical finding, eventually occurs in most involved joints. Symmetric involvement of small hand joints (especially proximal interphalangeal and metacarpophalangeal), foot joints (metatarsophalangeal), wrists, elbows, and ankles is typical, but initial manifestations may occur in any joint.

Psoriasis is a chronic skin disease, characterized by scaling and inflammation. Psoriasis affects 1.5 to 2 percent of the United States population, or almost 5 million people. It occurs in all age groups and about equally in men and women. People with psoriasis suffer discomfort, restricted motion of joints, and emotional distress. When psoriasis develops, patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis.

When skin is wounded, a wound-healing program is triggered, also known as regenerative maturation. Lesional psoriasis is characterized by cell growth in this alternate growth program. In many ways, psoriatic skin is similar to skin healing from a wound or reacting to a stimulus such as infection, where the keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

The exact cause of psoriasis in humans is not known, although it is generally accepted that it has a genetic component, and a recent study has established that it has an autoimmune component. Whether a person actually develops psoriasis is hypothesized to depend on something “triggering” its appearance. Examples of potential “trigger factors” include systemic infections, injury to the skin (the Koebner phenomenon), vaccinations, certain medications, and intramuscular injections or oral steroid medications. The chronic skin inflammation of psoriasis is associated with hyperplastic epidermal keratinocytes and infiltrating mononuclear cells, including CD4+ memory T cells, neutrophils and macrophages.

SLE. Systemic lupus erythematosus (SLE) is an autoimmune disease characterized by polyclonal B cell activation, which results in a variety of anti-protein and non-protein autoantibodies (see Kotzin et al. (1996) Cell 85:303-306 for a review of the disease). These autoantibodies form immune complexes that deposit in multiple organ systems, causing tissue damage. SLE is a difficult disease to study, having a variable disease course characterized by exacerbations and remissions. For example, some patients may demonstrate predominantly skin rash and joint pain, show spontaneous remissions, and require little medication. The other end of the spectrum includes patients who demonstrate severe and progressive kidney involvement (glomerulonephritis) that requires therapy with high doses of steroids and cytotoxic drugs such as cyclophosphamide.

Multiple factors may contribute to the development of SLE. Several genetic loci may contribute to susceptibility, including the histocompatibility antigens HLA-DR2 and HLA-DR3. The polygenic nature of this genetic predisposition, as well as the contribution of environmental factors, is suggested by a moderate concordance rate for identical twins, of between 25 and 60%.

Many causes have been suggested for the origin of autoantibody production. Proposed mechanisms of T cell help for anti-dsDNA antibody secretion include T cell recognition of DNA-associated protein antigens such as histones and recognition of anti-DNA antibody-derived peptides in the context of class II MHC. The class of antibody may also play a factor. In the hereditary lupus of NZB/NZW mice, cationic IgG2a anti-double-stranded (ds) DNA antibodies are pathogenic. The transition of autoantibody secretion from IgM to IgG in these animals occurs at the age of about six months, and T cells may play an important role in regulating the IgG production.

Disease manifestations result from recurrent vascular injury due to immune complex deposition, leukothrombosis, or thrombosis. Additionally, cytotoxic antibodies can mediate autoimmune hemolytic anemia and thrombocytopenia, while antibodies to specific cellular antigens can disrupt cellular function. An example of the latter is the association between anti-neuronal antibodies and neuropsychiatric SLE.

The present invention provides compositions and methods for characterizing, diagnosing and treating autoimmune disease(s) or other inflammatory disease(s). In particular, the present invention provides an TKI Responsive Signature and TKI Responsive Signature profiles associated with autoimmune disease(s) or other inflammatory disease(s), as well as novel markers or combination of markers useful for identifying diseases and individual patients likely to response to TKI therapy.

### Autoimmune or Other Inflammatory Disease Markers

The present invention provides markers whose expression is specifically altered in autoimmune or other inflammatory disease (e.g. up regulated or down regulated). Such markers or combination of markers find use in the diagnosis and characterization and alteration (e.g., therapeutic targeting) of various autoimmune diseases (e.g. systemic sclerosis, rheumatoid arthritis etc) or other inflammatory diseases. The markers comprising a TKI Responsive Signature predictive of responsiveness to TKI treatment are provided in Tables 2 and 3. While these tables provide gene names, it is noted that the present invention contemplates the use of the nucleic acid sequences as well as the proteins or peptides encoded thereby, as well as fragments of the nucleic acid and peptides, in the diagnostic and therapeutic methods and compositions of the present invention.

### Autoimmune Disease or Inflammatory Disease TKI Gene Signature

The present invention provides the means and methods for classifying patients afflicted with an autoimmune disease or other inflammatory disease based upon the profiling of autoimmune or other inflammatory disease samples by comparing a gene expression profile of an autoimmune disease or other inflammatory disease sample from a patient to a TKI Responsive Signature. This invention identifies an autoimmune disease or other inflammatory disease signature(s) that are predictors of response to TKI treatment and progression of disease. The microarray data of the present invention identifies autoimmune or other inflammatory disease markers likely to play a role in autoimmune or other inflammatory disease development, progression, and/or maintenance while also identifying a TKI Responsive Signature useful in identifying patients afflicted with an autoimmune or other inflammatory disease into classes or categories of either low and non-responsive or likely or responsive to TKI therapy. Classification based on the detection of differentially expressed polynucleotides and/or proteins that comprise a TKI Responsive Signature profile when compared to a TKI Responsive Signature can be used to predict clinical course, predict sensitivity to TKI treatment, guide selection of an appropriate TKI therapy, and monitor treatment response. Furthermore, following the development of therapeutics targeting such markers, detection of TKI Responsive Signatures described in detail below will allow the identification of patients likely to benefit from such therapeutics.

As described herein, the invention employs methods for clustering genes into gene expression profiles by determining their expression levels in two different cell or tissue samples. The invention further envisions using these gene profiles as compared to a TKI Responsive Signature to predict clinical outcome including, for example, therapeutic response to a TKI, disease progression and death. The microarray data of the present invention identifies gene profiles comprising similarly and differentially expressed genes between two tissue samples, one a test sample and one a reference sample, including between autoimmune or inflammatory disease cells or tissue, and control cells or tissue. These broad gene expression profiles can then be further refined, filtered, and subdivided into gene signatures based on various different criteria including, but not limited to, fold expression change, statistical analyses (e.g. Significance Analysis of Microarrys (SAM), Prediction Analysis of Microarrays (PAM)), biological function (e.g. cell cycle regulators, transcription factors, proteases, etc.), some therapeutic targets (e.g. functional pathways, matrix and vascular remodeling, immune signaling, growth factor signaling), identified expression in additional patient samples, and ability to predict clinical response to TKI therapy.

Thus certain embodiments of the present invention, the genes differentially expressed in autoimmune or other inflammatory disease cells versus control cells include TKI Responsive Signatures. Tyrosine kinase (TK)-related genes include any, all or a subset of genes that become altered in expression (activated or repressed) as a result of or in association with the activation of specific TKs. TK-related genes with statistically increased or decreased expression in autoimmune or other inflammatory disease cells could comprise an autoimmune or inflammatory disease TKI Responsive Signature. Alternatively, all genes above or below a certain fold expression change could represent an autoimmune or other inflammatory disease TKI Responsive Signature. For example, all TK-related genes with a 1 fold or more reduced (or elevated, or both) expression in autoimmune or other inflammatory disease cells can comprise one autoimmune or other inflammatory disease TKI Responsive Signature, all TK-related genes with a 2 fold or more reduced (or elevated, or both) expression in autoimmune or other inflammatory disease cells can comprise another autoimmune or other inflammatory disease TKI Responsive Signature, and so on. In some embodiments, the genes differentially expressed in autoimmune or inflammatory disease cells or tissue versus control cells or tissue are filtered by using statistical analysis. For example, all genes with elevated (or reduced, or both) expression based on Significance Analysis of Microarrays (SAM) analysis with a false discovery rate less than 5% can comprise one autoimmune or other inflammatory disease TKI Responsive Signature. Furthermore, gene expression analysis of independent patient samples or different cell lines can be compared to any TKI Responsive Signature generated as described above. An autoimmune or other inflammatory disease TKI Responsive Signature can be modified, for example, by calculating individual phenotype association indices as described to increase or maintain the predictive power of a given autoimmune or other inflammatory disease TKI Responsive Signature. In addition an autoimmune or other inflammatory disease TKI Responsive Signature can be further narrowed or expanded gene by gene by excluding or including genes subjectively (e.g. inclusion of a some therapeutic target or exclusion of a gene included in another gene signatures).

In further embodiments, a broad gene expression profile such as those generated by DNA array analyses of the present invention can be further refined, filtered, or subdivided into gene signatures based on two or more different criteria. In some embodiments of the present invention the genes differentially expressed in autoimmune or other inflammatory disease cells versus control cells are subdivided into different autoimmune or other inflammatory disease TKI Responsive Signature based on their fold expression change as well as their biological function. The generated TKI Responsive Signature is then compared against gene expression analysis from independent patient populations (referred to as the patient datasets), including datasets deposited in NCBI's Gene Expression Omnibus (GEO; http://www.ncbi.nlm.nih.gov/geo/) with examples below. In certain embodiments, the genes differentially expressed in autoimmune or other inflammatory disease cells versus control cells are divided into different autoimmune or other inflammatory disease TKI Responsive Signature based on their fold expression change and by statistical analysis. An alternative approach is to quantify the similarity of a gene profile to a reference response profile. The Pearson correlation of the averaged expression pattern with the reference response profile is then calculated. The Pearson correlation data allows the sample to be assigned as having a positive correlation to the responder response profile, or as being anti-correlated with responder response profile.

A scaled approach may also be taken to the data analysis. Pearson correlation of the expression values of the response profile of a sample to the reference response profile centroid results in a quantitative score reflecting the response profile for each sample. The higher the correlation value, the more the sample resembles the reference, responder profile. A negative correlation value indicates the opposite behavior and higher expression of the non-responder profile. The threshold for the two classes can be moved up or down from zero depending on the clinical goal.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

The data may be subjected to non-supervised hierarchical clustering to reveal relationships among profiles. For example, hierarchical clustering may be performed, where the Pearson correlation is employed as the clustering metric. Clustering of the correlation matrix, e.g. using multidimensional scaling, enhances the visualization of functional homology similarities and dissimilarities. Multidimensional scaling (MDS) can be applied in one, two or three dimensions.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. Such data may be used for a variety of purposes, such as drug discovery, analysis of interactions between cellular components, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

Patient classification. The invention provides for methods of classifying patients according to their response to a therapy of interest, particularly to a tyrosine kinase inhibitor, e.g. imatinib, or an analog or mimetic thereof including other PDGFR, Kit and Abl TKIs or PDFGR, Kit and Fms TKIs. The methods of the invention can be carried out using any suitable probe for detection of a gene product that is differentially expressed in a patient sample associated with an autoimmune disease or other inflammatory disease. For example, mRNA (or cDNA generated from mRNA) expressed from a response profile gene can be detected using polynucleotide probes. In another example, the response profile gene product is a polypeptide, which polypeptides can be detected using, for example, antibodies that specifically bind such polypeptides or an antigenic portion thereof.

The present invention relates to methods and compositions useful in design of rational therapy, and the selection of patients for therapy. The term expression profile is used broadly to include a genomic expression profile, e.g., an expression profile of mRNAs, or a proteomic expression profile, e.g., an expression profile of one or more different proteins. Profiles may be generated by any convenient means for determining differential gene expression between two samples, e.g. quantitative hybridization of mRNA, labeled mRNA, amplified mRNA, cRNA, etc., quantitative PCR, ELISA for protein quantitation, and the like. A subject or patient sample, e.g., cells or collections thereof, e.g., tissues, is assayed. Samples are collected by any convenient method, as known in the art.

In certain embodiments, the expression profile obtained is a genomic or nucleic acid expression profile, where the amount or level of one or more nucleic acids in the sample is determined. In these embodiments, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained.

The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, using standard protocols, where cell types or tissues from which such nucleic acids may be generated include any tissue in which the expression pattern of the to be determined phenotype exists. Cells may be cultured prior to analysis.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative. Alternatively, non-array based methods for quantitating the levels of one or more nucleic acids in a sample may be employed, including quantitative PCR, and the like.

Where the expression profile is a protein expression profile, any convenient protein quantitation protocol may be employed, where the levels of one or more proteins in the assayed sample are determined. Representative methods include, but are not limited to; proteomic arrays, flow cytometry, standard immunoassays, etc.

Following obtainment of the expression profile from the sample being assayed, the expression profile is compared with a reference or control profile to classify the patient as a responder or non-responder. A reference or control profile is provided, or may be obtained by empirical methods from samples of cells exposed to imatinib. In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The difference values, i.e. the difference in expression may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. A statistical analysis step is then performed to obtain the weighted contribution of the set of predictive genes, as described above.

The classification is probabilistically defined, where the cut-off may be empirically derived. In one embodiment of the invention, a probability of about 0.4 may be used to distinguish between quiescent and induced patients, more usually a probability of about 0.5, and may utilize a probability of about 0.6 or higher. A “high” probability may be at least about 0.75, at least about 0.7, at least about 0.6, or at least about 0.5. A “low” probability may be not more than about 0.25, not more than 0.3, or not more than 0.4. In many embodiments, the above-obtained information about the cell/tissue being assayed is employed to predict whether a host, subject or patient should be treated with a therapy of interest and to optimize the dose therein.

### Reagents and Kits

Also provided are reagents and kits thereof for practicing one or more of the above-described methods. The subject reagents and kits thereof may vary greatly. Reagents of interest include reagents specifically designed for use in production of the above described expression profiles of response profile genes.

One type of such reagent is an array of probe nucleic acids in which response profile genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In certain embodiments, the number of genes that are from that is represented on the array is at least 10, at least 15, at least 20, at least 25, at least 30, at least 35, at least 40, at least 45, up to including all of the response profile genes, preferably utilizing the top ranked set of genes.

Another type of reagent that is specifically tailored for generating expression profiles of response profile genes is a collection of gene specific primers that is designed to selectively amplify such genes, for use in quantitative PCR and other quantitation methods. Gene specific primers and methods for using the same are described in U.S. Pat. No. 5,994,076, the disclosure of which is herein incorporated by reference. Of particular interest are collections of gene specific primers that have primers for is at least 10, at least 15, at least 20, at least 25, at least 30, at least 35, at least 40, at least 45, up to including all of the response profile genes. The subject gene specific primer collections may include only response profile genes, or they may include primers for additional genes.

The kits of the subject invention may include the above described arrays and/or gene specific primer collections. The kits may further include a software package for statistical analysis of one or more phenotypes, and may include a reference database for calculating the probability of susceptibility. The kit may include reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Assessment of Patient Outcomes

Patient outcomes and Responder status may be assessed using imaging-based criteria such as radiographic scores, clinical and laboratory criteria. Multiple different imaging, clinical and laboratory criteria and scoring systems have been and are being developed to assess disease activity and response to therapy in systemic sclerosis, rheumatoid arthritis, systemic lupus erythematosus, Crohn's disease, and many other autoimmune or other inflammatory diseases.

In rheumatoid arthritis, response to therapy is conventionally measured using the American College of Rheumatology (ACR) Criteria. The ACR response criteria are a composite score comprising clinical (swollen joint count, tender joint count, physician and patient response assessment, and health assessment questionnaire), and laboratory (acute phase response) parameters; level of improvement is reported as an ACR20 (20%), ACR50 (50%) or ACR70 (70%) response, which indicates percent change (improvement) from the baseline score. A number of clinical trails based on which the anti-TNFa agents infliximab (Remicade™) etanercept (Enbrel™) and adalimumab (Humira™) were approved to treat human RA utilized ACR response rates as a primary outcome measure.

Responses in rheumatoid arthritis many also be assessed using other response criteria, such as the Disease Activity Score (DAS), which takes into account both the degree of improvement and the patient's current situation. The DAS has been shown to be comparable in validity to the ACR response criteria in clinical trials. The definitions of satisfactory and unsatisfactory response, in accordance with the original DAS and DAS28. The DAS28 is an index consisting of a 28 tender joint count, a 28 swollen joint count, ESR (or CRP), and an optional general health assessment on a visual analogue scale (range 0-100) (Clinical and Experimental Rheumatology, 23(Suppl. 39):S93-99, 2005). DAS28 scores are being used for quantification of response mostly in European trials of (early) rheumatoid arthritis such as the COBRA or BeST studies.

Radiographic measures for response in RA include both conventional X-rays (plain films), and more recently magnetic resonance (MR) imaging, computed tomography (CT), ultrasound and other imaging modalities are being utilized to monitor RA patients for disease progression. Such techniques are used to evaluate patients for inflammation (synovitis), joint effusions, cartilage damage, bony erosions and other evidence of joint damage. Methotrexate, anti-TNFalpha (TNFa) agents and DMARD combinations have been demonstrated to reduce development of bony erosions and other measures of joint inflammation and destruction in RA patients. In certain cases, such as with anti-TNFa agents, healing of bony erosions has been observed.

For response to therapy in systemic lupus erythematosus there exist a variety of scoring systems including the Ropes system, the National Institutes of Health [NIH] system, the New York Hospital for Special Surgery system, the British Isles Lupus Assessment Group [BILAG] scale, the University of Toronto SLE Disease Activity Index [SLE-DAI], and the Systemic Lupus Activity Measure [SLAM] (Arthritis and Rheumatism, 32(9):1107-18, 1989). The BILAG assessment consists of 86 questions; some based on the patient's history, some on examination findings and others on laboratory results. The questions are grouped under eight headings: General (Gen), Mucocutaneous (Muc), Neurological (Cns), Musculoskeletal (Msk), Cardiovascular and Respiratory (Car), Vasculitis (Vas), Renal (Ren), and Haematological (Hae). Based on the answers, a clinical score is calculated. The SLEDAI is a weighted, cumulative index of lupus disease activity.

Crohn's disease activity may be measured using the Crohn's disease activity index (CDAI) (Gastroenterology 70:439-444, 1976). The CDAI is based on the 1. Number of liquid or very soft stools in one week, 2. Sum of seven daily abdominal pain ratings: (0=none, 1=mild, 2=moderate, 3=severe), 3. Sum of seven daily ratings of general well-being: (0=well, 1=slightly below par, 2=poor, 3=very poor, 4=terrible), 4. Symptoms or findings presumed related to Crohn's disease (arthritis or arthralgia, iritis or uveitis, erythema nodosum, pyoderma gangrenosum, apththous stomatitis, anal fissure, fistula or perirectal abscess, other bowel-related fistula, febrile (fever) episode over 100 degrees during past week), 5. Taking Lomotil or opiates for diarrhea, 6. Abnormal mass, and 7. Hematocrit [(Typical−Current)×6]. Other criteria and scoring systems may also be used.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications and patent applications cited in this specification are herein incorporated by reference as if each individual publication or patent application were specifically and individually indicated to be incorporated by reference.

### EXPERIMENTAL

### Example 1

### Response to a PDGFR, Kit and Abl TKI in Systemic Sclerosis

Systemic sclerosis (SSc) is an autoimmune disease in which the tyrosine kinases platelet derived growth factor receptor (PDGFR) and Abl contribute to the fibrosis and vasculopathy of the skin and internal organs. We describe two patients with early diffuse SSc who experienced reductions in cutaneous sclerosis in response to therapy with the PDGFR, Kit and Abl tyrosine kinase inhibitor imatinib mesylate.

Imatinib mesylate (Gleevec, Novartis, East Hanover, N.J.) is a small molecule that antagonizes specific tyrosine kinases. We describe herein two patients with early diffuse SSc who experienced clinical improvement in response to imatinib therapy and provide evidence that both c-Abl and PDGFR are targets of imatinib in scleroderma skin.

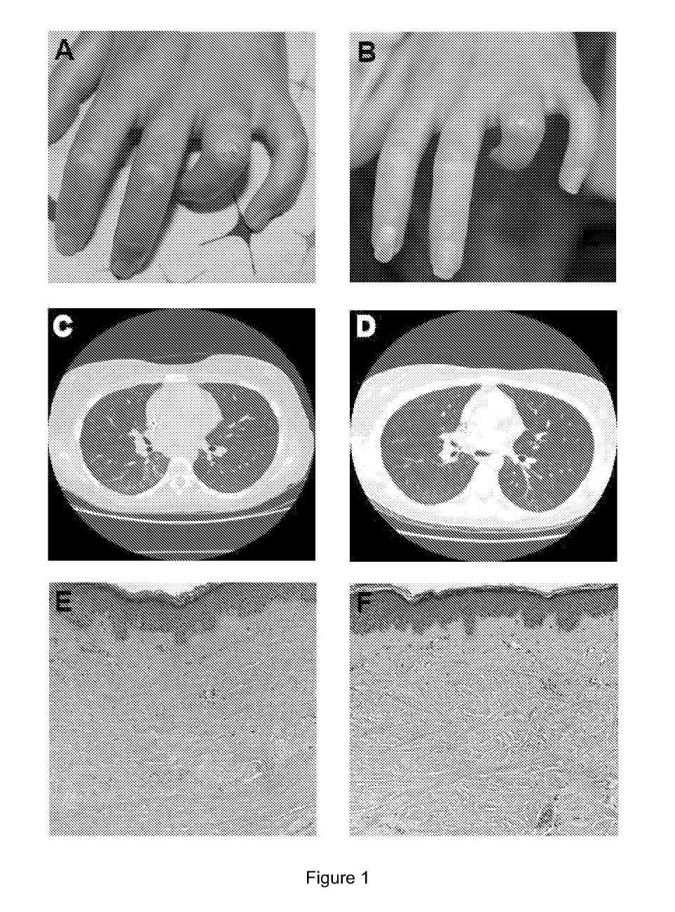

Patient 1. A 24-year old female with a 3-year history of diffuse SSc presented with increasing tightness of her skin and shortness of breath. The patient had a history of severe Raynaud's phenomenon and digital ulcerations (FIG. 1A) despite bilateral sympathectomies and treatment with multiple vasodilators. She suffered from arthritis requiring chronic prednisone at 10 mg daily. The patient had noticed increasing dyspnea on exertion and a high resolution computed tomography (HRCT) of the chest showed bibasilar ground glass opacities (FIG. 1C) consistent with interstitial lung disease (ILD). Pulmonary function tests showed a forced vital capacity (FVC) of 48% predicted and a diffusion capacity of carbon monoxide (DLCO) of 62% predicted. A transthoracic echocardiogram revealed a small pericardial effusion, but normal right ventricular systolic pressure (RVSP). The patient was intolerant to intravenous immunoglobulins and mycophenolate mofetil. She declined cyclophosphamide therapy and was referred to our center for a trial of imatinib.

Prior to initiating therapy, the patient's modified Rodnan skin thickness score (MRSS) was 36 (scale 0-51) and she had nine digital ulcers. Her complete blood count, comprehensive metabolic panel, creatine kinase, and urinalysis were within normal limits. C-reactive protein (CRP) level was 2.8 mg/dL (normal<0.5 mg/dL). A skin biopsy demonstrated thickened, closely packed collagen bundles with an average dermal thickness of 2.81 mm (FIG. 1E).

After three months of imatinib at 100 mg orally twice daily, the patient reported softening of her skin, increased joint mobility, and decreased shortness of breath. Physical examination revealed a MRSS of 21 and four digital ulcers (FIG. 1B). CRP had normalized to 0.2 mg/dL and the patient had been able to taper her prednisone to 5 mg daily. HRCT showed resolution of the interstitial changes (FIG. 1D) and a repeat TTE showed no evidence of a pericardial effusion. Repeat PFTs showed a slight improvement in her FVC to 52% predicted, but a decline in DLCO to 54% predicted. A repeat skin biopsy showed more widely spaced, thinner collagen bundles with an average dermal thickness of 2.31 mm (FIG. 1F).

Lesional skin biopsies of the upper extremities (upper arm or forearm) were obtained at baseline and during therapy after three months of therapy for histologic, immunohistochemical, and mRNA profiling via DNA array analyses. The protocol was approved by the institutional review board at Stanford University School of Medicine, and all patients provided written informed consent.

Patient 2. A 62-year old female with newly diagnosed diffuse SSc presented to our clinic with progressive cutaneous sclerosis. The patient had a 2-year history of Raynaud's phenomenon and noted increasing tightening of her skin over the previous 6 months. Initial therapies included benazepril for her Raynaud's and moderate doses of prednisone and methotrexate (12.5 mg/week) for her skin disease. The patient did not tolerate corticosteroid therapy and was referred to our center for investigational treatment with imatinib.

At initial evaluation, the patient had prominent capillary dilation and drop-out on nailfold capillaroscopy and her skin examination revealed a MRSS of 36. Her complete blood count, comprehensive metabolic panel, creatine kinase, urinalysis, and sedimentation rate were within normal limits. She had no evidence of ILD on HRCT of the chest and her PFTs were unremarkable. A baseline TTE showed a normal ejection fraction and an RVSP of 35 mmHg with a small pericardial effusion.

After 6 months of imatinib at 200 mg orally daily, the patient had noticed improvement in her skin tightening. Her Raynaud's worsened in severity during the winter season, but she did not develop any digital ulcers. On physical examination, her MRSS had improved to 20. Her PFTs and HRCT remained stable, and her TTE showed an RVSP of 23 mmHg and resolution of the pericardial effusion.

Lesional skin biopsies of the upper extremities (upper arm or forearm) were obtained at baseline and during therapy after one month of therapy for histologic, immunohistochemical, and mRNA profiling by DNA array analyses. The protocol was approved by the institutional review board at Stanford University School of Medicine, and all patients provided written informed consent.

### Example 2

### Effect of PDGFR, Kit and Abl TKI Therapy on Tyrosine Kinase Pathways In Vivo

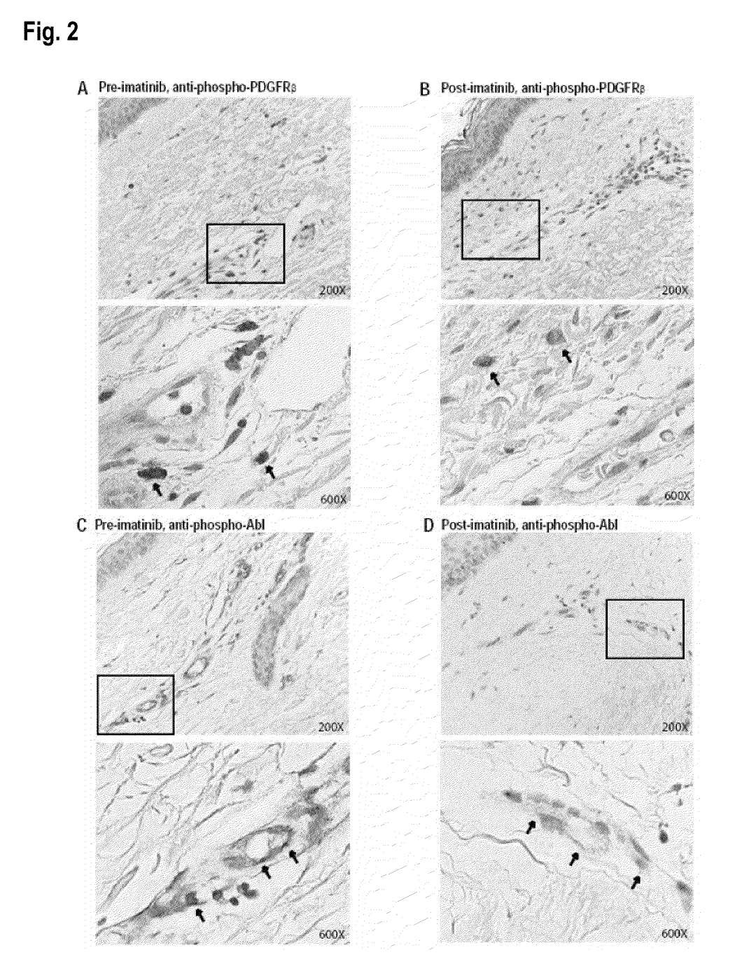

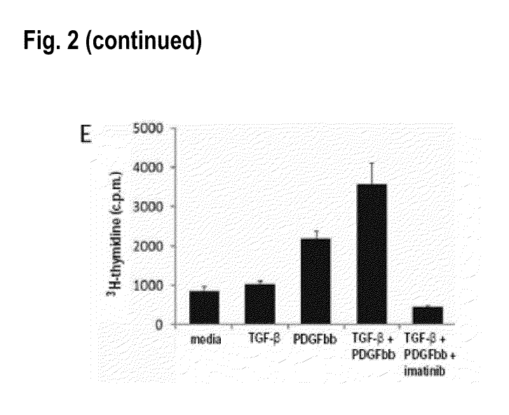

We performed immunohistochemical analysis on serial skin biopsies obtained pre-treatment and 1+ months following initiation of PDGFR, Kit and Abl TKI therapy with imatinib in Example 1. Tissue from skin biopsies was fixed in formalin and paraffin embedded. Sections were stained with antibodies specific for the phosphorylated (activated) states of the tyrosine kinases PDGFRb and c-Abl. An anti-phospho-PDGFRb antibody strongly stained dermal cells with fibroblast-like morphology in the pre-treatment sample (FIG. 2A), and there was a significant decrease in staining 1 month following initiation of imatinib therapy (FIG. 2B). Anti-phospho-Abl antibodies stained dermal vessels in the pre-treatment samples (FIG. 2C), and there was a significant decrease in staining 1 month following initiation of therapy (FIG. 2D).

Thus, immunohistochemistry demonstrated high levels of phospho-PDGFRbin dermal fibroblasts and phospho-Abl in vascular structures in pre-treatment skin biopsy samples, and reductions in phospho-PDGFRband phospho-Abl following initiation of imatinib therapy (FIG. 2A-D). Imatinib binds to the ATP-binding pockets to inhibit phosphorylation of the tyrosine kinases PDGFRband Abl, and these results suggest that imatinib-mediated inhibition of the activation of PDGFRband Abl is associated with the clinical benefit observed.

These results demonstrate that patients with SSc possessed high levels of phosphorylated (activated) PDGFRb and c-Abl in their pre-imatinib treatment samples, and treatment with the TKI imatinib is associated with a significant reduction in levels of phosphorylated PDGFRb and c-Abl.

### Example 3

### Effect of PDGFR, Kit and Abl TKI Therapy on Tyrosine Kinase Pathways In Vitro

Imatinib inhibits PDGF and TGF-β induced SSc fibroblast proliferation. To assess the ability of imatinib to inhibit PDGF and TGF-b induced fibroblast proliferation, titration curves for TGF-β and PDGF stimulation of SSc fibroblast proliferation were generated. Concentrations of TGF-β (0.5 ng/ml) and PDGF (10 ng/ml) that submaximally stimulated SSc fibroblast proliferation were selected and used alone, in combination, or in combination with imatinib (1 mM) to stimulate SSc fibroblast lines (FIG. 2E). As compared to the low level proliferation induced by PDGF or TGF-b alone, co-stimulation with PDGF and TGF-b synergistically induced SSc fibroblast proliferation (FIG. 2E; the increase in proliferation of the co-stimulated fibroblasts was two-times higher than the sum of the increases in proliferation observed with the individual stimuli). Imatinib completely abrogated SSc fibroblast proliferation induced by PDGF and TGF-\tilde{β}.

We thus demonstrated that PDGF and TGF-β0 each stimulate proliferation of SSc fibroblasts, while co-stimulation with PDGF+TGF-β synergistically induced proliferation. Addition of 1 mM imatinib, a concentration achieved in human dosing, inhibited the proliferation induced by PDGF+TGF-β (FIG. 2E). These data provide further evidence suggesting that aberrant activation of PDGFRb and Abl contribute to the pathogenesis of SSc, and that imatinib could provide benefit by inhibiting activation of these tyrosine kinases. Fibroblasts from patients with SSc have recently been shown to express increased levels of c-Kit, another tyrosine kinase potently inhibited by imatinib and that could play a significant role in the pathogenesis of SSc. The ability of imatinib to simultaneously inhibit multiple tyrosine kinase pathways involved in the pathogenesis of SSc likely contributes to the clinical benefit observed. Further, the effects in SSc were observed with lower doses of imatinib relative to those typically used to treat cancers. This may be due to the involvement of wild-type kinases in the pathogenesis of systemic sclerosis that are effectively inhibited at low doses of imatinib, while higher doses are needed to inhibit cancer cell growth mediated by mutated and aberrantly overexpressed kinases.

These results demonstrate that PDGFRb and Abl likely play a central role in the pathogenesis of SSc, and that inhibition of their activity using the TKI imatinib provides benefit in SSc.

### Example 4

### Identification of TKI Responsive Signature that Predicts Clinical Outcome in Systemic Sclerosis

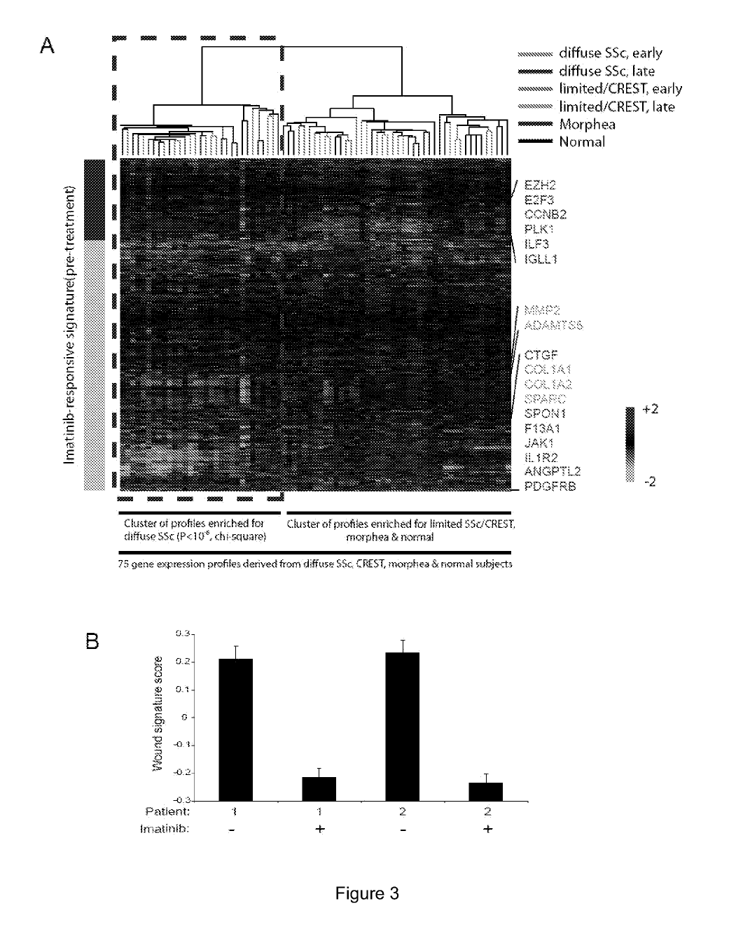

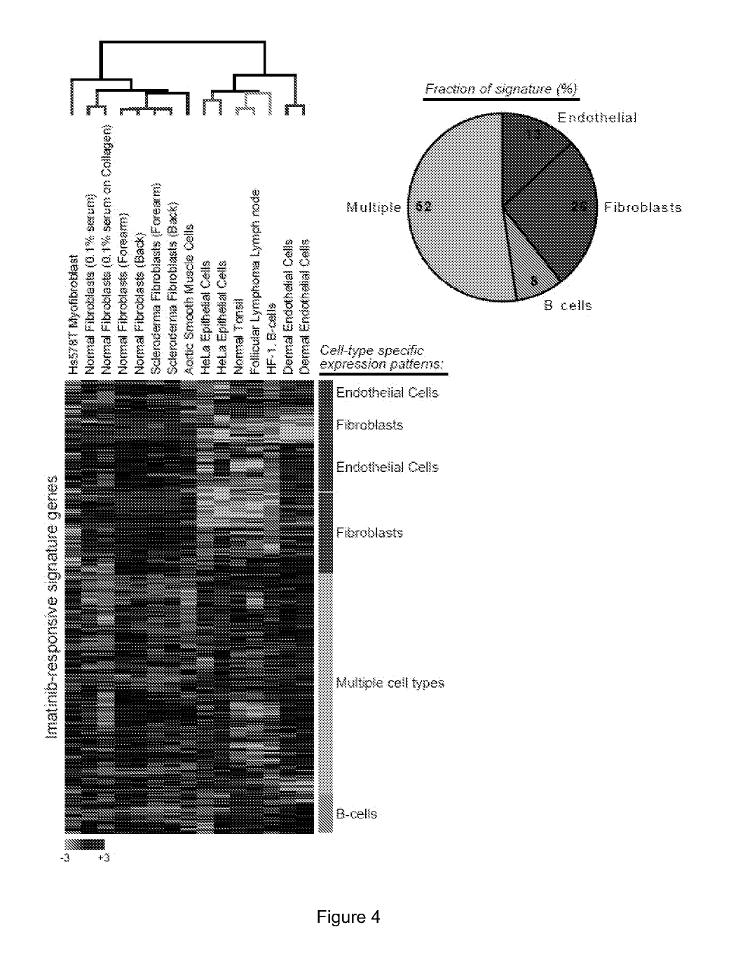

Identification of an imatinib-responsive gene signature. To gain further insights into the molecular mechanisms of imatinib action, we determined the global gene expression profiles of lesional skin before and after imatinib treatment. Comparison of gene expression patterns in the two patients before and after imatinib revealed a consistent set of 1032 genes, comprising a TKI Responsive Signature, that were changed by TKI therapy in both patients (FDR<0.001) (Table 2). To test whether the TKI Responsive Signature gene targets of imatinib in SSc, as defined in these two patients, may be generalizable to other patients with SSc or other fibrotic diseases, we interrogated the pattern of activation of the TKI Responsive Signature in a database of 75 gene expression profiles of SSc and control samples. We found that both early and late (≦ or >3 years in duration, respectively) diffuse SSc tended to express the TKI Responsive Signature, whereas most samples of normal skin, morphea, and limited SSc/CREST did not (FIG. 3A; P<10⁻⁸, chi-square).

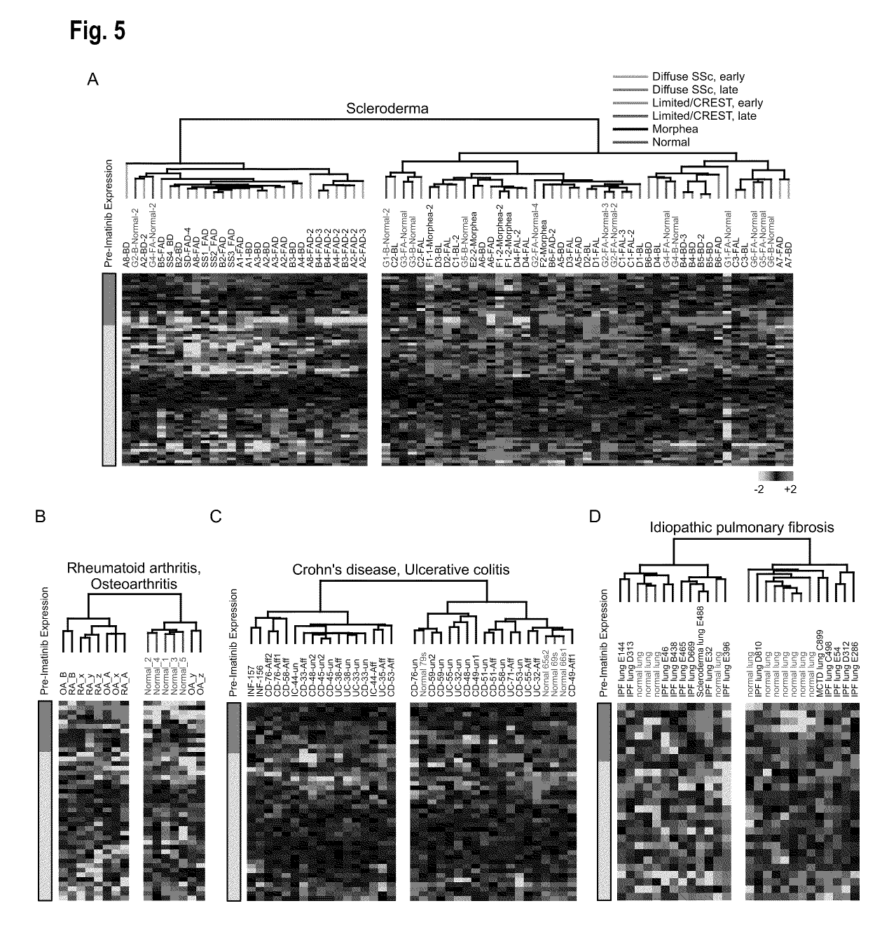

To determine which cell types may be contributing to the gene expression changes associated with TKI therapy, using previously published data and methodology we compared the TKI Responsive Signature to the gene expression profiles of 11 individual cell types that are likely to be present in skin. These 11 comparison cell types include normal and SSc fibroblasts, myofibroblasts, T and B cells, epithelial cells, and endothelial cells. This analysis suggests that about half of the expression changes can be attributed to one of three single cell types, including fibroblasts, endothelial cells and B cells, while the rest are likely expressed in multiple cell types (FIG. 5).

We characterized the global gene expression profiles in SSc skin before and after TKI (imatinib) treatment (FIG. 3). Because the post-treatment sample from patient 2 was obtained one month into imatinib treatment and before obvious clinical improvement, this gene expression signature may reflect the primary response of SSc to imatinib, rather than secondary changes associated with disease resolution. We identified a TKI Responsive Signature with genes involved in multiple functional pathways, including genes involved in cell proliferation, matrix and vascular remodeling, immune signaling, and growth factor signaling. The TKI Responsive Signature expression pattern was also specifically and frequently dysregulated in both early and late diffuse SSc. Importantly, consistent with the hypothesis that PDGF signaling may be activated in SSc, a TKI Responsive Signature of the transcriptional response of fibroblasts to serum, a principle component of which is PDGF, was induced in both SSc samples and substantially reduced by imatinib treatment (FIG. 3B; P<0.01, Student's t-test).

While case reports can highlight new disease entities or treatment options, they are traditionally limited by the uncertainty of general applicability. Here we use genomic profiling to bridge this gap. We identified a TKI Responsive Signature from our SSc patients undergoing experimental therapy with the TKI imatinib. By comparison with a larger database of gene profiles from patients with fibrosing disorders, we found that the majority of patients with diffuse SSc, but not limited SSc or morphea, also exhibit the same transcriptional TKI Responsive Signature. Diffuse SSc patients who express this TKI Responsive Signature may benefit clinically from imatinib.

Global transcriptional analysis of skin using oligonucleotide microarrays. Total RNA was extracted from snap frozen skin biopsies (taken adjacent to those processed for paraffin embedding) before and after TKI (imatinib) treatment using Qiagen RNeasy fibrous tissue kit. RNA was amplified using the Ambion Amino Allyl MessageAmp II aRNA kit. Amplified skin RNA (labeled with Cy5) and amplified Stratagene Human Universal Reference RNA (labeled with Cy3) were competitively hybridized to human exon evidence-based oligonucleotide (HEEBO) microarrays in duplicate as described.

Genes selected for analysis had fluorescent hybridization signal at least 1.5-fold over local background in either Cy5 or Cy3 channel and had technically adequate data in at least 75% of experiments. Genes were analyzed by mean value centering within the dataset for each patient. TKI Responsive Signature genes were identified using Significance Analysis of Microarrays with false discovery rate (FDR)<0.001. Samples were scored for their similarity to the transcriptional response of fibroblasts to serum as described by Chang et al. (2005) Proc Natl Acad Sci USA 2005; 102(10):3738-3743. The database of 75 SSc and control gene expression profiles are described by Milano et al. PLoS ONE. 2008; 3(7):e2696, and include 75 microarray analyses on 61 skin biopsies from 34 subjects, including samples from 18 patients with diffuse SSc, 7 with limited SSc, 3 with morphea, and 6 healthy controls. 817 of 1050 TKI Responsive Signature genes were successfully mapped in the SSc database using EntrezGene ID, and their pattern of expression was analyzed by unsupervised hierarchical clustering, revealing two distinct clusters. The TKI Responsive Signature expression pattern was similar to one of the clusters, which was highly enriched for diffuse SSc samples (29 of the 31 gene expression profiles in this cluster were derived from diffuse SSc, P<10⁻⁸, chi-square). The TKI Responsive Signature is provided in Table 2.

The cell types that express the genes contained in the TKI Responsive Signature derived in FIG. 3 and presented in Table 2 were further classified and characterized. The TKI-responsive gene expression signature was derived from gene expression profiles of 11 individual cell types that are likely to be present in skin. Using UniGene ID to convert the genes, 485 of 1050 imatinib-responsive genes were identified. The sequence of the gene or gene product may be found in public databases, including those listed in the table. Specific information regarding the genetic sequence at a particular date is also available from these databases. Imatinib-responsive genes that are specifically expressed in a given cell type are highlighted on the right. The percentages of the genes specifically expressed in fibroblasts, endothelial cells, B-cells, or multiple cell types are provided.

TABLE 2

| Gene Symbol | EntrezGene | UniGene | Imatinib response |
|---|---|---|---|
| ILF3 | 3609 | Hs.465885 | Repressed |
| DTYMK | 1841 | Hs.471873 | Repressed |
| WHSC1 | 7468 | Hs.113876 | Repressed |
| SLC12A9 | 56996 | Hs.521087 | Repressed |
| C1orf63 | 57035 | Hs.259412 | Repressed |
| NOM1 | 64434 | Hs.15825 | Repressed |
| ACSM3 | 6296 | Hs.653192 | Repressed |
| PMS1 | 5378 | Hs.111749 | Repressed |
| TMEM8 | 58986 | Hs.288940 | Repressed |
| IGKC | 3514 | | Repressed |
| ENOSF1 | 55556 | Hs.369762 | Repressed |
| SMA5 | 11042 | Hs.529793 | Repressed |
| ZNF331 | 55422 | Hs.185674 | Repressed |
| PPY2 | 23614 | Hs.20588 | Repressed |
| PDCD11 | 22984 | Hs.239499 | Repressed |
| ZNF518 | 9849 | Hs.657337 | Repressed |
| LOC442585 | 442585 | | Repressed |
| DFNB31 | 25861 | Hs.93836 | Repressed |
| HRBL | 3268 | | Repressed |
| CTPS | 1503 | Hs.473087 | Repressed |
| BYSL | 705 | Hs.106880 | Repressed |
| MGC15912 | 84972 | Hs.656176 | Repressed |
| PLK1 | 5347 | Hs.592049 | Repressed |
| UHRF1 | 29128 | Hs.108106 | Repressed |
| NOL5A | 10528 | | Repressed |
| NFS1 | 9054 | Hs.194692 | Repressed |
| ncRNA_U22_7 | | | Repressed |
| SLC25A37 | 51312 | Hs.122514 | Repressed |
| ANKHD1 | 54882 | | Repressed |
| HSUP1 | 441951 | | Repressed |
| CCNB1 | 891 | Hs.23960 | Repressed |
| KIDINS220 | 57498 | Hs.9873 | Repressed |
| ZNF587 | 84914 | Hs.288995 | Repressed |
| THOC3 | 84321 | Hs.548868 | Repressed |
| KIAA1804 | 84451 | Hs.547779 | Repressed |
| E2F3 | 1871 | Hs.269408 | Repressed |
| SERPINA1 | 5265 | Hs.525557 | Repressed |
| BIRC5 | 332 | Hs.514527 | Repressed |
| B4GALNT4 | 338707 | Hs.148074 | Repressed |
| HS3ST3A1 | 9955 | Hs.462270 | Repressed |
| TYMS | 7298 | Hs.592338 | Repressed |
| EIF2B5 | 8893 | Hs.283551 | Repressed |
| SPBC24 | 147841 | Hs.381225 | Repressed |
| PASK | 23178 | Hs.397891 | Repressed |
| CA9 | 768 | Hs.63287 | Repressed |
| KIF20A | 10112 | Hs.73625 | Repressed |
| C1orf107 | 27042 | Hs.194754 | Repressed |
| TGFB1 | 7040 | Hs.645227 | Repressed |
| LMNB2 | 84823 | Hs.538286 | Repressed |
| LOC391322 | 391322 | Hs.568022 | Repressed |
| SLC37A4 | 2542 | Hs.132760 | Repressed |
| CIT | 11113 | Hs.119594 | Repressed |
| CHEK2 | 11200 | Hs.291363 | Repressed |
| AYTL2 | 79888 | Hs.368853 | Repressed |
| MRPL35 | 51318 | Hs.433439 | Repressed |
| HN1 | 51155 | Hs.532803 | Repressed |
| UBE2S | 27338 | Hs.396393 | Repressed |
| HNRPH1 | 3187 | Hs.604001 | Repressed |
| ZWINT | 11130 | Hs.591363 | Repressed |
| RNASEH2A | 10535 | Hs.532851 | Repressed |
| C1orf63 | 57035 | Hs.259412 | Repressed |
| LOC389049 | 389049 | | Repressed |
| C9orf45 | 81571 | Hs.657064 | Repressed |
| CTA-246H3.1 | 91353 | Hs.567636 | Repressed |
| ANKHD1 | 54882 | | Repressed |
| KIAA0922 | 23240 | Hs.205572 | Repressed |
| TNFRSF6B | 8771 | Hs.434878 | Repressed |
| CARD14 | 79092 | Hs.655729 | Repressed |
| DTX3L | 151636 | Hs.518201 | Repressed |
| FLJ20273 | 54502 | Hs.518727 | Repressed |
| ASF1B | 55723 | Hs.26516 | Repressed |
| HBG2 | 3048 | Hs.302145 | Repressed |
| HNRPA2B1 | 3181 | Hs.487774 | Repressed |
| SERPINF2 | 5345 | Hs.159509 | Repressed |
| TAPBP | 6892 | Hs.370937 | Repressed |
| HBG1 | 3047 | Hs.295459 | Repressed |
| CKS2 | 1164 | Hs.83758 | Repressed |
| LOC375251 | 375251 | Hs.535591 | Repressed |
| KIF11 | 3832 | Hs.8878 | Repressed |
| HNRPH1 | 3187 | Hs.202166 | Repressed |
| ZNF207 | 7756 | Repressed | RNA |
| TAP1 | 6890 | Hs.352018 | Repressed |
| IGLC1 | 3537 | Repressed | RNA |
| WIPI2 | 26100 | Hs.122363 | Repressed |
| IGLL1 | 3543 | Hs.348935 | Repressed |
| EST_AI791445 | 28566 | | Repressed |
| SFRS2 | 6427 | Hs.584801 | Repressed |
| MICAL1 | 64780 | Hs.33476 | Repressed |
| ATP1B1 | 481 | Hs.291196 | Repressed |
| EZH2 | 2146 | Hs.444082 | Repressed |
| ncRNA_U25_0 | | | Repressed |
| GAGE7B | 26748 | Hs.460641 | Repressed |
| HIST1H4C | 8364 | Hs.46423 | Repressed |
| HERC4 | 26091 | Hs.51891 | Repressed |
| UBE2C | 11065 | Hs.93002 | Repressed |
| ANKRD36 | 375248 | Hs.541894 | Repressed |
| AURKA | 6790 | Hs.250822 | Repressed |
| PUS7 | 54517 | Hs.520619 | Repressed |
| TCF25 | 22980 | Hs.415342 | Repressed |
| LOC389221 | 389221 | | Repressed |
| FKSG24 | 84769 | Hs.515254 | Repressed |
| SFRS14 | 10147 | Hs.515271 | Repressed |
| PTTG1 | 9232 | Hs.350966 | Repressed |
| RHAG | 6005 | Hs.120950 | Repressed |
| SLC38A5 | 92745 | Hs.195155 | Repressed |
| SNRP70 | 6625 | Hs.467097 | Repressed |
| JARID1A | 5927 | Hs.654806 | Repressed |
| TRIM73 | 375593 | Hs.632307 | Repressed |
| PRPF38B | 55119 | Hs.342307 | Repressed |
| PVALB | 5816 | Hs.295449 | Repressed |
| LRWD1 | 222229 | Hs.274135 | Repressed |
| GRK6 | 2870 | Hs.235116 | Repressed |
| CCDC34 | 91057 | Hs.143733 | Repressed |
| RBM26 | 64062 | Hs.558528 | Repressed |
| TACC3 | 10460 | Hs.104019 | Repressed |
| LOC339047 | 339047 | Hs.513373 | Repressed |
| DENND2D | 79961 | Hs.557850 | Repressed |
| PRPF38A | 84950 | Hs.5301 | Repressed |
| UBE2T | 29089 | Hs.5199 | Repressed |
| DMXL2 | 23312 | Hs.511386 | Repressed |
| BTN3A2 | 11118 | Hs.376046 | Repressed |
| CDCA8 | 55143 | Hs.524571 | Repressed |
| MATK | 4145 | Hs.631845 | Repressed |
| CORO1A | 11151 | Hs.415067 | Repressed |
| RNU22 | 9304 | Hs.523739 | Repressed |
| MARK3 | 4140 | Hs.35828 | Repressed |
| MKI67 | 4288 | Hs.80976 | Repressed |
| MT1F | 4494 | Hs.513626 | Repressed |
| SDCCAG1 | 9147 | Hs.655964 | Repressed |
| PAXIP1 | 22976 | Hs.443881 | Repressed |
| SP140 | 11262 | Hs.632549 | Repressed |
| ING3 | 54556 | Hs.489811 | Repressed |
| GART | 2618 | Hs.473648 | Repressed |
| TTC13 | 79573 | Hs.424788 | Repressed |
| HBA1 | 3039 | Hs.449630 | Repressed |
| NUSAP1 | 51203 | Hs.615092 | Repressed |
| KIAA0286 | 23306 | Hs.591040 | Repressed |
| EST_AI791445 | 28566 | | Repressed |
| APOBEC3B | 9582 | Hs.226307 | Repressed |
| PRODH2 | 58510 | Hs.515366 | Repressed |
| NUDC | 10726 | Hs.263812 | Repressed |
| ZNF292 | 23036 | Hs.590890 | Repressed |
| C20orf72 | 92667 | Hs.320823 | Repressed |
| NOL1 | 4839 | Hs.534334 | Repressed |
| ZNF275 | 10838 | Hs.348963 | Repressed |
| LOC441019 | 441019 | Hs.568282 | Repressed |
| TRAF3 | 7187 | Hs.510528 | Repressed |
| LOC441260 | 441260 | | Repressed |
| TSEN54 | 283989 | Hs.655875 | Repressed |
| ZNF234 | 10780 | Hs.334586 | Repressed |
| EIF4A1::Y11161 | 1973 | Hs.129673 | Repressed |
| GNB1L | 54584 | Hs.105642 | Repressed |
| PRO0628 | 29053 | Hs.592136 | Repressed |
| ncRNA_6_583 | | | Repressed |
| SLC38A2 | 54407 | | Repressed |
| ENO3 | 2027 | Hs.224171 | Repressed |
| EST_AA935786 | | | Repressed |
| DONSON | 29980 | Hs.436341 | Repressed |
| C1orf79 | 85028 | Hs.632377 | Repressed |
| GOLGA8B | 440270 | | Repressed |
| LIG1 | 3978 | Hs.1770 | Repressed |
| H2AFX | 3014 | Hs.477879 | Repressed |
| UBE2S | 27338 | Hs.396393 | Repressed |
| SURF5 | 6837 | Hs.78354 | Repressed |
| EST_AA032084 | | | Repressed |
| C6orf111 | 25957 | Hs.520287 | Repressed |
| SPG7 | 6687 | Hs.185597 | Repressed |
| GOLGA8G | 283768 | Hs.525714 | Repressed |
| ncRNA_U81_0 | | | Repressed |
| RNU47 | 26802 | | Repressed |
| LOC91316 | 91316 | Hs.148656 | Repressed |
| CDC25B | 994 | Hs.153752 | Repressed |
| C1orf79 | 85028 | | Repressed |
| C9orf140 | 89958 | Hs.19322 | Repressed |
| FLJ11184 | 55319 | Hs.267446 | Repressed |
| EST_AI334107 | | | Repressed |
| E2F2 | 1870 | Hs.194333 | Repressed |
| TGFB1 | 7040 | Hs.645227 | Repressed |
| IKBKB | 3551 | Hs.656458 | Repressed |
| FNBP4 | 23360 | Hs.6834 | Repressed |
| MT1G | 4495 | Hs.433391 | Repressed |
| AARSL | 57505 | Hs.158381 | Repressed |
| LOC440470 | 440470 | Hs.568305 | Repressed |
| NAGK | 55577 | Hs.7036 | Repressed |
| ZP3 | 7784 | Hs.656137 | Repressed |
| WDR46 | 9277 | Hs.520063 | Repressed |
| TMC6 | 11322 | Hs.632227 | Repressed |
| PTTG3 | 26255 | Hs.545401 | Repressed |
| FAM111A | 63901 | Hs.150651 | Repressed |
| IKBKE | 9641 | Hs.321045 | Repressed |
| RNASE2 | 6036 | Hs.728 | Repressed |
| TRAF5 | 7188 | Hs.523930 | Repressed |
| DENND4B | 9909 | Hs.632480 | Repressed |
| ANKRD52 | 283373 | Hs.524506 | Repressed |
| FASTKD1 | 79675 | Hs.529276 | Repressed |
| NARG1 | 80155 | Hs.555985 | Repressed |
| LRAP | 64167 | Hs.591249 | Repressed |
| DAPP1 | 27071 | Hs.436271 | Repressed |
| KCNG1 | 3755 | Hs.118695 | Repressed |
| CEP110 | 11064 | Hs.653263 | Repressed |
| SYT13 | 57586 | Hs.436643 | Repressed |
| FKBP5 | 2289 | Hs.407190 | Repressed |
| ALAS2 | 212 | Hs.522666 | Repressed |
| TncRNA | 283131 | | Repressed |
| NUSAP1 | 51203 | Hs.615092 | Repressed |
| MT1G | 4495 | Hs.433391 | Repressed |
| CDK5RAP3 | 80279 | Hs.20157 | Repressed |
| NT5DC3 | 51559 | Hs.48428 | Repressed |
| KIAA0226 | 9711 | Hs.478868 | Repressed |
| LSG1 | 55341 | Hs.518505 | Repressed |
| TRIB2 | 28951 | Hs.467751 | Repressed |
| NUP210 | 23225 | Hs.475525 | Repressed |
| ZNF232 | 7775 | Hs.279914 | Repressed |
| HYLS1 | 219844 | Hs.585071 | Repressed |
| CLK1 | 1195 | Hs.433732 | Repressed |
| SRPK2 | 6733 | Hs.285197 | Repressed |
| DDX55 | 57696 | Hs.286173 | Repressed |
| HBA2 | 3040 | Hs.449630 | Repressed |
| C6orf173 | 387103 | | Repressed |
| SCO2 | 9997 | Hs.658057 | Repressed |
| KIAA1245 | 149013 | | Repressed |
| NUP35 | 129401 | Hs.180591 | Repressed |
| GNL3L | 54552 | Hs.29055 | Repressed |
| PAG1 | 55824 | Hs.266175 | Repressed |
| CKS2 | 1164 | Hs.83758 | Repressed |
| MLF1IP | 79682 | Hs.481307 | Repressed |
| HSP90AA2 | 3324 | Hs.523560 | Repressed |
| HSD17B7 | 51478 | Hs.492925 | Repressed |
| PSRC1 | 84722 | Hs.405925 | Repressed |
| PRO1580 | 55374 | Hs.631799 | Repressed |
| RAB8A | 4218 | Hs.631641 | Repressed |
| U2AF1L2 | 8233 | Hs.171909 | Repressed |
| THEM4 | 117145 | Hs.164070 | Repressed |
| TRNM | 4569 | | Repressed |
| DEF6 | 50619 | Hs.15476 | Repressed |
| METTL6 | 131965 | Hs.149487 | Repressed |
| TBC1D10C | 374403 | Hs.534648 | Repressed |
| ZNF278 | 23598 | Hs.517557 | Repressed |
| DDX26B | 203522 | Hs.496829 | Repressed |
| ZNF182 | 7569 | Hs.189690 | Repressed |
| PPHLN1 | 51535 | Hs.444157 | Repressed |
| DCLRE1C | 64421 | Hs.656065 | Repressed |
| C16orf53 | 79447 | Hs.655071 | Repressed |
| EFHD2 | 79180 | Hs.465374 | Repressed |
| CGI-09 | 51605 | Hs.128791 | Repressed |
| AP1GBP1 | 11276 | Hs.655178 | Repressed |
| RNF34 | 80196 | Hs.292804 | Repressed |
| RFWD3 | 55159 | Hs.567525 | Repressed |
| MCM7 | 4176 | Hs.438720 | Repressed |
| NOP5/NOP58 | 51602 | Hs.471104 | Repressed |
| TUSC4 | 10641 | Hs.437083 | Repressed |
| TK1 | 7083 | Hs.515122 | Repressed |
| FNBP4 | 23360 | Hs.6834 | Repressed |
| DDX27 | 55661 | Hs.65234 | Repressed |
| HSPC111 | 51491 | Hs.652195 | Repressed |
| ILF3 | 3609 | Hs.465885 | Repressed |
| MLL5 | 55904 | | Repressed |
| C11orf30 | 56946 | Hs.352588 | Repressed |
| RAPGEF6 | 51735 | Hs.483329 | Repressed |
| FLJ10154 | 55082 | Hs.508644 | Repressed |
| GLT25D1 | 79709 | Hs.418795 | Repressed |
| XM_499148 | 441443 | | Repressed |
| MXD3 | 83463 | Hs.653158 | Repressed |
| SNAP29 | 9342 | Hs.108002 | Repressed |
| ATF7IP2 | 80063 | Hs.513343 | Repressed |
| PHF20L1 | 51105 | Hs.304362 | Repressed |
| TFEC | 22797 | Hs.125962 | Repressed |
| CCDC41 | 51134 | Hs.279209 | Repressed |
| STK4 | 6789 | Hs.472838 | Repressed |
| DNAJC1 | 64215 | Hs.499000 | Repressed |
| MYBL2 | 4605 | Hs.179718 | Repressed |
| NOL5A | 10528 | Hs.376064 | Repressed |
| AKAP1 | 8165 | Hs.463506 | Repressed |
| KIAA1794 | 55215 | Hs.513126 | Repressed |
| CDC20 | 991 | Hs.524947 | Repressed |
| GUSBL2 | 375513 | Hs.561539 | Repressed |
| CCNB2 | 9133 | Hs.194698 | Repressed |
| MCM5 | 4174 | Hs.517582 | Repressed |
| ARL4C | 10123 | Hs.111554 | Repressed |
| FUS | 2521 | Hs.513522 | Repressed |
| ARHGEF1 | 9138 | Hs.631550 | Repressed |
| SFRS7 | 6432 | Hs.309090 | Repressed |
| GLYCTK | 132158 | Hs.415312 | Repressed |
| FANCL | 55120 | Hs.631890 | Repressed |
| EZH2 | 2146 | Hs.444082 | Repressed |
| RRS1 | 23212 | Hs.71827 | Repressed |
| CHORDC1 | 26973 | Hs.22857 | Repressed |
| RBM39 | 9584 | Hs.282901 | Repressed |
| SLC36A1 | 206358 | Hs.269004 | Repressed |
| USP52 | 9924 | Hs.273397 | Repressed |
| XM_376575 | 401307 | | Repressed |
| ncRNA_mir- | | | Induced |
| 320_12 | | | |
| EST_AA885292 | | | Induced |
| SLC9A9 | 285195 | Hs.302257 | Induced |
| PDCD6IP | 10015 | Hs.475896 | Induced |
| ADSSL1 | 122622 | Hs.592327 | Induced |
| TRIM16 | 10626 | Hs.123534 | Induced |
| RAMP2 | 10266 | Hs.514193 | Induced |
| DCTN1 | 1639 | Hs.516111 | Induced |
| ROBO4 | 54538 | Hs.524121 | Induced |
| ANKRD38 | 163782 | Hs.283398 | Induced |
| LOC285812 | 285812 | Hs.593631 | Induced |
| ACACB | 32 | Hs.234898 | Induced |
| POF1B | 79983 | Hs.267038 | Induced |
| NDFIP1 | 80762 | Hs.9788 | Induced |
| KIAA1913 | 114801 | Hs.591341 | Induced |
| FAM10A3 | 144638 | | Induced |
| CCDC35 | 387750 | Hs.647273 | Induced |
| SLC18A2 | 6571 | Hs.654476 | Induced |
| SMPDL3A | 10924 | Hs.486357 | Induced |
| LGI2 | 55203 | Hs.12488 | Induced |
| PHB2 | 11331 | Hs.504620 | Induced |
| EST_AA991868 | | | Induced |
| CDSN | 1041 | Hs.556031 | Induced |
| BTBD6 | 90135 | Hs.7367 | Induced |
| CCL23 | 6368 | Hs.169191 | Induced |
| TSPYL4 | 23270 | Hs.284141 | Induced |
| MGC59937 | 375791 | Hs.512469 | Induced |
| LOC388135 | 388135 | | Induced |
| LCE1A | 353131 | Hs.534645 | Induced |
| FBLN1 | 2192 | Hs.24601 | Induced |
| KRT10 | 3858 | Hs.99936 | Induced |
| NDEL1 | 81565 | Hs.372123 | Induced |
| TOP3B | 8940 | Hs.436401 | Induced |
| SCEL | 8796 | Hs.534699 | Induced |
| EST_AA416628 | | | Induced |
| ANKRD50 | 57182 | Hs.480694 | Induced |
| FADS2 | 9415 | Hs.502745 | Induced |
| PIP | 5304 | Hs.99949 | Induced |
| ELMOD1 | 55531 | Hs.495779 | Induced |
| KIAA1377 | 57562 | Hs.156352 | Induced |
| HSPA12A | 259217 | Hs.372457 | Induced |
| PALM | 5064 | Hs.631841 | Induced |
| SDC1 | 6382 | Hs.224607 | Induced |
| RKHD1 | 399664 | Hs.436495 | Induced |
| FBLN1 | 2192 | Hs.24601 | Induced |
| ZFHX4 | 79776 | Hs.458973 | Induced |
| ELN | 2006 | Hs.647061 | Induced |
| RGMB | 285704 | Hs.526902 | Induced |
| LCE2C | 353140 | Hs.553713 | Induced |
| PCP4 | 5121 | Hs.80296 | Induced |
| MYO10 | 4651 | Hs.43334 | Induced |
| PPP1R14C | 81706 | Hs.486798 | Induced |
| PPP1R15A | 23645 | Hs.631593 | Induced |
| STK24 | 8428 | Hs.508514 | Induced |
| MCC | 4163 | Hs.593171 | Induced |
| CSDA | 8531 | Hs.221889 | Induced |
| PCTK2 | 5128 | Hs.506415 | Induced |
| PCBP2 | 5094 | Hs.546271 | Induced |
| TMEM45A | 55076 | Hs.658956 | Induced |
| HMGA1 | 3159 | Hs.518805 | Induced |
| DSP | 1832 | Hs.519873 | Induced |
| CRNN | 49860 | Hs.242057 | Induced |
| ANTXR1 | 84168 | Hs.165859 | Induced |
| TGM1 | 7051 | Hs.508950 | Induced |
| F3 | 2152 | Hs.62192 | Induced |
| EFNA1 | 1942 | Hs.516664 | Induced |
| RALBP1 | 10928 | Hs.528993 | Induced |
| LARGE | 9215 | Hs.474667 | Induced |
| RUNX1T1 | 862 | Hs.368431 | Induced |
| HSPC159 | 29094 | Hs.372208 | Induced |
| ANKRD15 | 23189 | Hs.306764 | Induced |
| SLPI | 6590 | Hs.517070 | Induced |
| FBLN1 | 2192 | Hs.24601 | Induced |
| K5B | 196374 | Hs.665267 | Induced |
| PEA15 | 8682 | Hs.517216 | Induced |
| CCRL1 | 51554 | Hs.310512 | Induced |
| RHOD | 29984 | Hs.15114 | Induced |
| RAB40C | 57799 | Hs.459630 | Induced |
| PARD6G | 84552 | Hs.654920 | Induced |
| ZNF185 | 7739 | Hs.16622 | Induced |
| SLCO4A1 | 28231 | Hs.235782 | Induced |
| KRT80 | 144501 | Hs.140978 | Induced |
| MYH9 | 4627 | Hs.474751 | Induced |
| TPM2 | 7169 | Hs.300772 | Induced |
| PLXDC2 | 84898 | Hs.658134 | Induced |
| PTPRF | 5792 | Hs.272062 | Induced |
| DHCR24 | 1718 | Hs.498727 | Induced |
| XM_165511 | 220832 | | Induced |
| RBM35A | 54845 | Hs.487471 | Induced |
| BPIL2 | 254240 | Hs.372939 | Induced |
| OR2A1 | 346528 | Hs.528398 | Induced |
| KIAA1614 | 57710 | Hs.647760 | Induced |
| KIF1B | 23095 | Hs.97858 | Induced |
| ARFRP1 | 10139 | Hs.389277 | Induced |
| SLC2A1 | 6513 | Hs.473721 | Induced |
| LEP7 | 353138 | | Induced |
| TMEM86A | 144110 | Hs.502100 | Induced |
| SH3BP4 | 23677 | Hs.516777 | Induced |
| TTC15 | 51112 | Hs.252713 | Induced |
| CYGB | 114757 | Hs.95120 | Induced |
| SIDT2 | 51092 | Hs.410977 | Induced |
| LOC116236 | 116236 | Hs.106510 | Induced |
| PLXNA4A | 57671 | Hs.511454 | Induced |
| TIAM1 | 7074 | Hs.517228 | Induced |
| ATOH8 | 84913 | Hs.135569 | Induced |
| LANCL2 | 55915 | Hs.655117 | Induced |
| DEGS2 | 123099 | Hs.159643 | Induced |
| S100A16 | 140576 | Hs.515714 | Induced |
| ECM2 | 1842 | Hs.117060 | Induced |
| ITPK1 | 3705 | Hs.308122 | Induced |
| BCL2L2 | 599 | Hs.410026 | Induced |
| SERPINB2 | 5055 | Hs.594481 | Induced |
| RPRC1 | 55700 | Hs.356096 | Induced |
| CLIC3 | 9022 | Hs.64746 | Induced |
| TNFRSF10D | 8793 | Hs.213467 | Induced |
| TUSC1 | 286319 | Hs.26268 | Induced |
| MAFF | 23764 | Hs.517617 | Induced |
| SEPT8 | 23176 | Hs.533017 | Induced |
| SCGB1D2 | 10647 | Hs.204096 | Induced |
| DKFZp667G2110 | 131544 | Hs.607776 | Induced |
| ANKRD47 | 256949 | Hs.591401 | Induced |
| ABHD9 | 79852 | Hs.156457 | Induced |
| MAF | 4094 | Hs.134859 | Induced |
| LOC283666 | 283666 | Hs.560343 | Induced |
| FRMD6 | 122786 | Hs.434914 | Induced |
| KLHDC3 | 116138 | Hs.412468 | Induced |
| POLR2L | 5441 | Hs.441072 | Induced |
| KLF11 | 8462 | Hs.12229 | Induced |
| RPS28 | 6234 | Hs.153177 | Induced |
| PVRL2 | 5819 | Hs.110675 | Induced |
| COX6A1 | 1337 | Hs.497118 | Induced |
| BMP7 | 655 | Hs.473163 | Induced |
| DNAJC14 | 85406 | Hs.505676 | Induced |
| EST_AA029434 | | | Induced |
| ELMOD2 | 255520 | Hs.450105 | Induced |
| DAG1 | 1605 | Hs.76111 | Induced |
| RNF141 | 50862 | Hs.44685 | Induced |
| PDLIM2 | 64236 | | Induced |
| IGFBP3 | 3486 | Hs.450230 | Induced |
| UBTD1 | 80019 | Hs.500724 | Induced |
| ID4 | 3400 | Hs.519601 | Induced |
| EXOC4 | 60412 | Hs.321273 | Induced |
| TMEM147 | 10430 | Hs.9234 | Induced |
| EST_AA252511 | | | Induced |
| KRT23 | 25984 | Hs.9029 | Induced |
| SLC29A4 | 222962 | Hs.4302 | Induced |
| COL1A1 | 1277 | Hs.172928 | Induced |
| FOXO3A | 2309 | Hs.220950 | Induced |
| IGFBP4 | 3487 | Hs.462998 | Induced |
| MAL2 | 114569 | Hs.201083 | Induced |
| RAI14 | 26064 | Hs.431400 | Induced |
| PIGT | 51604 | Hs.437388 | Induced |
| PTGDS | 5730 | Hs.446429 | Induced |
| PER1 | 5187 | Hs.445534 | Induced |
| OGN | 4969 | Hs.109439 | Induced |
| TYRO3 | 7301 | Hs.381282 | Induced |
| ENDOD1 | 23052 | Hs.167115 | Induced |
| KLF4 | 9314 | Hs.376206 | Induced |
| RWDD1 | 51389 | Hs.532164 | Induced |
| DDT | 1652 | Hs.632781 | Induced |
| DEGS1 | 8560 | Hs.299878 | Induced |
| CHMP4C | 92421 | Hs.183861 | Induced |
| SCARA3 | 51435 | Hs.128856 | Induced |
| LDOC1 | 23641 | Hs.45231 | Induced |
| IL1R2 | 7850 | Hs.25333 | Induced |
| GJA1 | 2697 | Hs.74471 | Induced |
| PSORS1C2 | 170680 | Hs.146824 | Induced |
| COX1 | 4512 | | Induced |
| EBPL | 84650 | Hs.433278 | Induced |
| SEC15L2 | 23233 | Hs.303454 | Induced |
| CGNL1 | 84952 | Hs.148989 | Induced |
| YPEL2 | 388403 | Hs.463613 | Induced |
| SNF1LK | 150094 | Hs.282113 | Induced |
| TUBB2A | 7280 | Hs.654543 | Induced |
| TMEM19 | 55266 | Hs.653275 | Induced |
| AACS | 65985 | Hs.656073 | Induced |
| ZDHHC9 | 51114 | Hs.193566 | Induced |
| CDKN1C | 1028 | Hs.106070 | Induced |
| RPS24 | 6229 | Hs.356794 | Induced |
| MAP3K9 | 4293 | Hs.445496 | Induced |
| SULT2B1 | 6820 | Hs.369331 | Induced |
| COL12A1 | 1303 | Hs.101302 | Induced |
| LCE5A | 254910 | Hs.516410 | Induced |
| PTP4A1 | 7803 | Hs.227777 | Induced |
| C9orf58 | 83543 | Hs.4944 | Induced |
| CTSF | 8722 | Hs.11590 | Induced |
| TIE1 | 7075 | Hs.78824 | Induced |
| LYPD5 | 284348 | Hs.44289 | Induced |
| GPR81 | 27198 | Hs.610873 | Induced |
| NOV | 4856 | Hs.235935 | Induced |
| SERPINH1 | 871 | Hs.596449 | Induced |
| ARHGAP10 | 79658 | Hs.368631 | Induced |
| MFSD5 | 84975 | Hs.654660 | Induced |
| STOX1 | 219736 | Hs.37636 | Induced |
| tcag7.981 | 221895 | Hs.368944 | Induced |
| LOC342897 | 342897 | Hs.451636 | Induced |
| AEBP1 | 165 | Hs.439463 | Induced |
| CXX1 | 8933 | Hs.522789 | Induced |
| C3orf28 | 26355 | Hs.584881 | Induced |
| DARC | 2532 | Hs.153381 | Induced |
| HYAL2 | 8692 | Hs.76873 | Induced |
| DAB2IP | 153090 | Hs.522378 | Induced |
| NFIA | 4774 | Hs.191911 | Induced |
| LCE1B | 353132 | Hs.375103 | Induced |
| ABHD12 | 26090 | Hs.441550 | Induced |
| RAB42P | 337996 | | Induced |
| KIF13B | 23303 | Hs.444767 | Induced |
| NMNAT3 | 349565 | Hs.208673 | Induced |
| ANKRD37 | 353322 | Hs.508154 | Induced |
| SERPINB8 | 5271 | Hs.368077 | Induced |
| PCDH1 | 5097 | Hs.79769 | Induced |
| TMEM88 | 92162 | Hs.389669 | Induced |
| ATP7A | 538 | Hs.496414 | Induced |
| ABCA12 | 26154 | Hs.134585 | Induced |
| SAMD4A | 23034 | Hs.98259 | Induced |
| FLJ21986 | 79974 | Hs.189652 | Induced |
| NAB1 | 4664 | Hs.570078 | Induced |
| RAI2 | 10742 | Hs.446680 | Induced |
| CAMK1D | 57118 | Hs.659517 | Induced |
| COL1A2 | 1278 | Hs.489142 | Induced |
| KIAA0494 | 9813 | Hs.100874 | Induced |
| SPON1 | 10418 | Hs.643864 | Induced |
| C20orf23 | 55614 | Hs.101774 | Induced |
| LPPR4 | 9890 | Hs.13245 | Induced |
| LOC124976 | 124976 | Hs.567664 | Induced |
| MAP1LC3A | 84557 | Hs.632273 | Induced |
| RNASE7 | 84659 | Hs.525206 | Induced |
| CDKN1A | 1026 | Hs.370771 | Induced |
| HSD11B1 | 3290 | Hs.195040 | Induced |
| GGTLA1 | 2687 | Hs.437156 | Induced |
| LOC199800 | 199800 | Hs.311193 | Induced |
| LOC440449 | 440449 | | Induced |
| IL1F5 | 26525 | Hs.516301 | Induced |
| ADCY4 | 196883 | Hs.443428 | Induced |
| TPM4 | 7171 | Hs.631618 | Induced |
| LOC439994 | 439994 | Hs.534856 | Induced |
| 39510 | 115123 | Hs.132441 | Induced |
| ACSS2 | 55902 | Hs.517034 | Induced |
| AXUD1 | 64651 | Hs.370950 | Induced |
| PARVA | 55742 | Hs.436319 | Induced |
| KIAA1467 | 57613 | Hs.132660 | Induced |
| NUDT16 | 131870 | Hs.591313 | Induced |
| PLS3 | 5358 | Hs.496622 | Induced |
| PPM1F | 9647 | Hs.112728 | Induced |
| B3GALT4 | 8705 | Hs.534375 | Induced |
| MPST | 4357 | Hs.248267 | Induced |
| CEBPD | 1052 | Hs.440829 | Induced |
| LOC145853 | 145853 | Hs.438385 | Induced |
| GAN | 8139 | Hs.112569 | Induced |
| KIAA0372 | 9652 | Hs.482868 | Induced |
| SASH1 | 23328 | Hs.193133 | Induced |
| CALM1 | 801 | Hs.282410 | Induced |
| COL5A2 | 1290 | Hs.445827 | Induced |
| RAI17 | 57178 | Hs.193118 | Induced |
| GATM | 2628 | Hs.75335 | Induced |
| PLLP | 51090 | Hs.632215 | Induced |
| KIAA2002 | 79834 | Hs.9587 | Induced |
| SLC44A1 | 23446 | Hs.573495 | Induced |
| KIAA1344 | 57544 | Hs.532609 | Induced |
| ANK3 | 288 | Hs.499725 | Induced |
| LCE1C | 353133 | Hs.516429 | Induced |
| KIF26A | 26153 | Hs.134970 | Induced |
| CHL1 | 10752 | Hs.148909 | Induced |
| PPFIBP1 | 8496 | Hs.172445 | Induced |
| EST_AA664003 | | | Induced |
| TEF | 7008 | Hs.181159 | Induced |
| MCC | 4163 | Hs.593171 | Induced |
| ZUBR1 | 23352 | Hs.148078 | Induced |
| PLVAP | 83483 | Hs.107125 | Induced |
| USP2 | 9099 | Hs.524085 | Induced |
| HSPA2 | 3306 | Hs.432648 | Induced |
| RAB6A | 5870 | Hs.503222 | Induced |
| ANKRD57 | 65124 | Hs.355455 | Induced |
| LOC441158 | 441158 | | Induced |
| PI16 | 221476 | Hs.25391 | Induced |
| MGLL | 11343 | Hs.277035 | Induced |
| TGFA | 7039 | Hs.170009 | Induced |
| GAS6 | 2621 | Hs.646346 | Induced |
| MYL9 | 10398 | Hs.504687 | Induced |
| MGC22014 | 200424 | Hs.516107 | Induced |
| DPYSL2 | 1808 | Hs.173381 | Induced |
| B3GNT5 | 84002 | Hs.208267 | Induced |
| PHPT1 | 29085 | Hs.409834 | Induced |
| TMEM16K | 55129 | Hs.656657 | Induced |
| PBX1 | 5087 | Hs.654412 | Induced |
| SESN1 | 27244 | Hs.591336 | Induced |
| COBLL1 | 22837 | Hs.470457 | Induced |
| MRPS27 | 23107 | Hs.482491 | Induced |
| ATP9A | 10079 | Hs.592144 | Induced |
| NMT2 | 9397 | Hs.60339 | Induced |
| PODXL | 5420 | Hs.16426 | Induced |
| PTPRM | 5797 | Hs.49774 | Induced |
| MIR16 | 51573 | Hs.512607 | Induced |
| VPS24 | 51652 | Hs.591582 | Induced |
| FBXL3 | 26224 | Hs.508284 | Induced |
| GNG12 | 55970 | Hs.431101 | Induced |
| MTMR2 | 8898 | Hs.181326 | Induced |
| FLJ20701 | 55022 | Hs.409352 | Induced |
| EST_AA463463 | | | Induced |
| BTD | 686 | Hs.517830 | Induced |
| NEDD9 | 4739 | Hs.37982 | Induced |
| ATP1A2 | 477 | Hs.34114 | Induced |
| MMRN2 | 79812 | Hs.524479 | Induced |
| GPAM | 57678 | Hs.42586 | Induced |
| LDB2 | 9079 | Hs.23748 | Induced |
| ARHGAP21 | 57584 | Hs.524195 | Induced |
| GALNT1 | 2589 | Hs.514806 | Induced |
| UBE2E2 | 7325 | Hs.475688 | Induced |
| CITED2 | 10370 | Hs.82071 | Induced |
| INSIG2 | 51141 | Hs.7089 | Induced |
| EFNB2 | 1948 | Hs.149239 | Induced |
| C1QTNF5 | 114902 | Hs.632102 | Induced |
| NMB | 4828 | Hs.386470 | Induced |
| NGRN | 51335 | Hs.513145 | Induced |
| SERPINB3 | 6317 | Hs.227948 | Induced |
| PSPHL | 8781 | Hs.536913 | Induced |
| BRP44 | 25874 | Hs.517768 | Induced |
| FEM1A | 55527 | Hs.515082 | Induced |
| TMEM99 | 147184 | Hs.353163 | Induced |
| CLDN5 | 7122 | Hs.505337 | Induced |
| RPS8 | 6202 | Hs.512675 | Induced |
| LAMC1 | 3915 | Hs.497039 | Induced |
| HSPB1 | 3315 | Hs.520973 | Induced |
| COL6A2 | 1292 | Hs.420269 | Induced |
| TCEAL4 | 79921 | Hs.194329 | Induced |
| SPFH1 | 10613 | Hs.150087 | Induced |
| TOB1 | 10140 | Hs.531550 | Induced |
| TPST2 | 8459 | Hs.655859 | Induced |
| KIAA1128 | 54462 | Hs.461988 | Induced |
| TNXB | 7148 | Hs.485104 | Induced |
| IDE | 3416 | Hs.500546 | Induced |
| SPAG1 | 6674 | Hs.591866 | Induced |
| HIG2 | 29923 | Hs.433213 | Induced |
| JAK1 | 3716 | Hs.207538 | Induced |
| LHX6 | 26468 | Hs.103137 | Induced |
| MORF4L1 | 10933 | Hs.374503 | Induced |
| C10orf10 | 11067 | Hs.93675 | Induced |
| CLDN4 | 1364 | Hs.647036 | Induced |
| EHBP1 | 23301 | Hs.271667 | Induced |
| PRSS23 | 11098 | Hs.25338 | Induced |
| LOC130678 | 130678 | | Induced |
| NR2F2 | 7026 | Hs.347991 | Induced |
| 39702 | 55752 | Hs.128199 | Induced |
| GTF2F2 | 2963 | Hs.654582 | Induced |
| PRDM1 | 639 | Hs.436023 | Induced |
| CD36 | 948 | Hs.120949 | Induced |
| STS | 412 | Hs.522578 | Induced |
| TWIST2 | 117581 | Hs.422585 | Induced |
| CCDC80 | 151887 | Hs.477128 | Induced |
| ITGB1 | 3688 | Hs.429052 | Induced |
| FSTL1 | 11167 | Hs.269512 | Induced |
| PJA2 | 9867 | Hs.483036 | Induced |
| NGFRAP1 | 27018 | Hs.448588 | Induced |
| RPL27 | 6155 | Hs.514196 | Induced |
| FLJ10357 | 55701 | Hs.35125 | Induced |
| TMEM23 | 259230 | Hs.654698 | Induced |
| CYB5A | 1528 | Hs.465413 | Induced |
| ATXN1 | 6310 | Hs.434961 | Induced |
| ENG | 2022 | Hs.76753 | Induced |
| FBXO45 | 200933 | Hs.518526 | Induced |
| NFE2L2 | 4780 | Hs.155396 | Induced |
| LMCD1 | 29995 | Hs.475353 | Induced |
| SLC39A6 | 25800 | Hs.79136 | Induced |
| CNN3 | 1266 | Hs.483454 | Induced |
| UBC | 7316 | Hs.520348 | Induced |
| BLMH | 642 | Hs.371914 | Induced |
| TCEAL8 | 90843 | Hs.389734 | Induced |
| H19 | 283120 | Hs.533566 | Induced |
| MESP1 | 55897 | Hs.447531 | Induced |
| FBXO28 | 23219 | Hs.64691 | Induced |
| MAOB | 4129 | Hs.654473 | Induced |
| EHD4 | 30844 | Hs.143703 | Induced |
| RAB18 | 22931 | Hs.406799 | Induced |
| C15orf48 | 84419 | Hs.112242 | Induced |
| SPTLC1 | 10558 | Hs.90458 | Induced |
| SEMA3G | 56920 | Hs.59729 | Induced |
| CD55 | 1604 | Hs.527653 | Induced |
| MMP2 | 4313 | Hs.513617 | Induced |
| CMKOR1 | 57007 | Hs.471751 | Induced |
| HSPB8 | 26353 | Hs.400095 | Induced |
| EPB41L2 | 2037 | Hs.486470 | Induced |
| CFD | 1675 | Hs.155597 | Induced |
| SORD | 6652 | Hs.878 | Induced |
| CALD1 | 800 | Hs.490203 | Induced |
| TCEAL1 | 9338 | Hs.95243 | Induced |
| PICALM | 8301 | Hs.163893 | Induced |
| FLJ36070 | 284358 | Hs.191815 | Induced |
| EBPL | 84650 | Hs.433278 | Induced |
| ACOT2 | 10965 | Hs.446685 | Induced |
| CXXC5 | 51523 | Hs.189119 | Induced |
| HTRA1 | 5654 | Hs.501280 | Induced |
| PPP3CA | 5530 | Hs.435512 | Induced |
| PSMB5 | 5693 | Hs.422990 | Induced |
| PLXND1 | 23129 | Hs.301685 | Induced |
| EBPL | 84650 | Hs.433278 | Induced |
| PEPD | 5184 | Hs.36473 | Induced |
| TSPAN31 | 6302 | Hs.632708 | Induced |
| RAB11A | 8766 | Hs.321541 | Induced |
| ALDH1A1 | 216 | Hs.76392 | Induced |
| H19 | 283120 | | Induced |
| CASK | 8573 | Hs.495984 | Induced |
| LANCL1 | 10314 | Hs.13351 | Induced |
| TJP1 | 7082 | Hs.510833 | Induced |
| APP | 351 | Hs.434980 | Induced |
| RPL41 | 6171 | Hs.112553 | Induced |
| CD1A | 909 | Hs.1309 | Induced |
| CYP51A1 | 1595 | Hs.417077 | Induced |
| PDGFRB | 5159 | Hs.509067 | Induced |
| MFAP4 | 4239 | Hs.296049 | Induced |
| RASD1 | 51655 | Hs.25829 | Induced |
| CYBRD1 | 79901 | Hs.221941 | Induced |
| NID2 | 22795 | Hs.369840 | Induced |
| CD24 | 934 | Hs.644105 | Induced |
| DBI | 1622 | Hs.78888 | Induced |
| PDE2A | 5138 | Hs.503163 | Induced |
| MTMR12 | 54545 | Hs.481836 | Induced |
| CTNNA1 | 1495 | Hs.534797 | Induced |
| DCN | 1634 | Hs.156316 | Induced |
| IGFBP5 | 3488 | Hs.369982 | Induced |
| ADAMTS5 | 11096 | Hs.58324 | Induced |
| LAMA4 | 3910 | Hs.654572 | Induced |
| LUM | 4060 | Hs.406475 | Induced |
| COQ2 | 27235 | Hs.144304 | Induced |
| TIMP2 | 7077 | Hs.633514 | Induced |
| C14orf112 | 51241 | Hs.137108 | Induced |
| C2orf30 | 27248 | Hs.438336 | Induced |
| FADS3 | 3995 | Hs.21765 | Induced |
| NID1 | 4811 | Hs.356624 | Induced |
| COX7B | 1349 | Hs.522699 | Induced |
| DPYSL3 | 1809 | Hs.519659 | Induced |
| GJA1 | 2697 | Hs.74471 | Induced |
| MME | 4311 | Hs.307734 | Induced |
| SH3BGRL2 | 83699 | Hs.302772 | Induced |
| SH3BP5 | 9467 | Hs.257761 | Induced |
| SPARC | 6678 | Hs.111779 | Induced |
| RNASE4 | 6038 | Hs.283749 | Induced |
| MOSC1 | 64757 | Hs.497816 | Induced |
| RAB6C | 84084 | Hs.591552 | Induced |
| HSDL2 | 84263 | Hs.59486 | Induced |
| F13A1 | 2162 | Hs.335513 | Induced |
| LAPTM4A | 9741 | Hs.467807 | Induced |
| TGFBR3 | 7049 | Hs.482390 | Induced |
| SC4MOL | 6307 | Hs.105269 | Induced |
| CES1 | 1066 | Hs.558865 | Induced |
| ANGPTL2 | 23452 | Hs.642746 | Induced |
| FABP7 | 2173 | Hs.26770 | Induced |
| UBL3 | 5412 | Hs.145575 | Induced |
| THY1 | 7070 | Hs.653181 | Induced |
| RBP4 | 5950 | Hs.50223 | Induced |
| GPRC5B | 51704 | Hs.148685 | Induced |
| MSMB | 4477 | Hs.255462 | Induced |
| RARRES2 | 5919 | Hs.647064 | Induced |
| ATP6V1H | 51606 | Hs.491737 | Induced |
| CDC42 | 998 | Hs.597524 | Induced |
| C3orf57 | 165679 | Hs.369104 | Induced |
| SQLE | 6713 | Hs.71465 | Induced |
| AKR7A2 | 8574 | Hs.571886 | Induced |
| PFKFB3 | 5209 | Hs.195471 | Induced |
| SOX18 | 54345 | Hs.8619 | Induced |
| MAPRE2 | 10982 | Hs.532824 | Induced |
| SPON2 | 10417 | Hs.302963 | Induced |
| AQP7 | 364 | Hs.455323 | Induced |
| GLTP | 51228 | Hs.381256 | Induced |
| YIPF3 | 25844 | Hs.440950 | Induced |
| YIF1A | 10897 | Hs.446445 | Induced |
| NEBL | 10529 | Hs.5025 | Induced |
| TMEPAI | 56937 | Hs.517155 | Induced |
| MBOAT2 | 129642 | Hs.467634 | Induced |
| FBLN1 | 2192 | Hs.24601 | Induced |
| CD99 | 4267 | Hs.495605 | Induced |
| DGAT2 | 84649 | Hs.334305 | Induced |
| SPTLC2L | 140911 | Hs.425023 | Induced |
| ATP5I | 521 | Hs.85539 | Induced |
| FBLN1 | 2192 | Hs.24601 | Induced |
| LRP1 | 4035 | Hs.162757 | Induced |
| EST_AA708719 | | | Induced |
| C10orf116 | 10974 | Hs.642660 | Induced |
| PER2 | 8864 | Hs.58756 | Induced |
| LOC442133 | 442133 | | Induced |
| TM9SF2 | 9375 | Hs.654824 | Induced |
| TMOD3 | 29766 | Hs.4998 | Induced |
| SERTAD2 | 9792 | Hs.591569 | Induced |
| EMP1 | 2012 | Hs.436298 | Induced |
| FLJ10986 | 55277 | Hs.444301 | Induced |
| PIGC | 5279 | Hs.188456 | Induced |
| MXI1 | 4601 | Hs.501023 | Induced |
| RETSAT | 54884 | Hs.440401 | Induced |
| CTGF | 1490 | Hs.591346 | Induced |
| LOC143381 | 143381 | Hs.388347 | Induced |
| AGTRL1 | 187 | Hs.438311 | Induced |
| ANKRD15 | 23189 | Hs.306764 | Induced |
| DBN1 | 1627 | Hs.130316 | Induced |
| THBS1 | 7057 | Hs.164226 | Induced |
| LOC400843 | 400843 | | Induced |
| PDK4 | 5166 | Hs.8364 | Induced |
| COL5A1 | 1289 | Hs.210283 | Induced |
| RASA4 | 10156 | Hs.530089 | Induced |
| COPG2 | 26958 | Hs.532231 | Induced |
| DUSP14 | 11072 | Hs.91448 | Induced |
| CTDP1 | 9150 | Hs.465490 | Induced |
| RSN | 6249 | Hs.524809 | Induced |
| FKBP9L | 360132 | Hs.446691 | Induced |
| SNX19 | 399979 | Hs.444024 | Induced |
| GPD1 | 2819 | Hs.524418 | Induced |
| FCGBP | 8857 | Hs.111732 | Induced |
| SERPING1 | 710 | Hs.384598 | Induced |
| APOD | 347 | Hs.522555 | Induced |
| CRY2 | 1408 | Hs.532491 | Induced |
| RPLP1 | 6176 | Hs.356502 | Induced |
| MPEG1 | 219972 | Hs.643518 | Induced |
| SYNE1 | 23345 | Hs.12967 | Induced |
| FBXO45 | 200933 | Hs.518526 | Induced |
| CHIC2 | 26511 | Hs.335393 | Induced |
| SPARCL1 | 8404 | Hs.62886 | Induced |
| COL3A1 | 1281 | Hs.443625 | Induced |
| C4orf18 | 51313 | Hs.567498 | Induced |
| LOC389305 | 389305 | Hs.567966 | Induced |
| C10orf57 | 80195 | Hs.169982 | Induced |
| HEBP2 | 23593 | Hs.486589 | Induced |
| KRT77 | 374454 | Hs.334989 | Induced |
| UBE2N | 7334 | Hs.524630 | Induced |
| TMEM54 | 113452 | Hs.534521 | Induced |
| EDNRA | 1909 | Hs.183713 | Induced |
| DYNLRB1 | 83658 | Hs.593920 | Induced |
| STEAP4 | 79689 | Hs.521008 | Induced |
| RGS5 | 8490 | Hs.24950 | Induced |
| GAB2 | 9846 | Hs.429434 | Induced |
| COL6A1 | 1291 | Hs.474053 | Induced |
| MSRB3 | 253827 | Hs.339024 | Induced |
| GALNT1 | 2589 | Hs.514806 | Induced |
| CIRBP | 1153 | Hs.501309 | Induced |
| EDG1 | 1901 | Hs.154210 | Induced |
| LRP10 | 26020 | Hs.525232 | Induced |
| EIIs1 | 222166 | Hs.200100 | Induced |
| TGFBR2 | 7048 | Hs.82028 | Induced |
| CTHRC1 | 115908 | Hs.405614 | Induced |
| LOC196264 | 196264 | Hs.15396 | Induced |
| AYP1 | 84153 | Hs.397010 | Induced |
| ADD1 | 118 | Hs.183706 | Induced |
| HSPB6 | 126393 | Hs.534538 | Induced |
| IRS2 | 8660 | Hs.442344 | Induced |
| AOC3 | 8639 | Hs.198241 | Induced |
| NDUFA3 | 4696 | Hs.198269 | Induced |
| FZD1 | 8321 | Hs.94234 | Induced |
| TINAGL1 | 64129 | Hs.199368 | Induced |
| SPTLC2L | 140911 | | Induced |
| AK095567 | 284014 | Hs.131035 | Induced |
| TMBIM1 | 64114 | Hs.591605 | Induced |
| CAV1 | 857 | Hs.74034 | Induced |
| CTSL2 | 1515 | Hs.660866 | Induced |
| CFH | 3075 | Hs.363396 | Induced |
| MMD | 23531 | Hs.463483 | Induced |
| FLJ20160 | 54842 | Hs.418581 | Induced |
| LMO2 | 4005 | Hs.34560 | Induced |
| EST_AA479967 | | | Induced |
| COX7B | 1349 | Hs.522699 | Induced |
| LYPLA1 | 10434 | Hs.435850 | Induced |
| DKFZP564M1416 | 25869 | | Induced |
| GPD1L | 23171 | Hs.82432 | Induced |
| GOLGA5 | 9950 | Hs.104320 | Induced |
| MGC4677 | 112597 | Hs.446688 | Induced |
| NCKAP1 | 10787 | Hs.603732 | Induced |
| GABARAPL1 | 23710 | Hs.524250 | Induced |
| LOC441114 | 441114 | Hs.519738 | Induced |
| PIR | 8544 | Hs.495728 | Induced |
| FYTTD1 | 84248 | Hs.277533 | Induced |
| TPD52L1 | 7164 | Hs.591347 | Induced |
| SURF4 | 6836 | Hs.512465 | Induced |
| H3F3B | 3021 | Hs.180877 | Induced |
| EST_AA447504 | | | Induced |
| CYFIP1 | 23191 | Hs.26704 | Induced |
| ATP5H | 10476 | Hs.514465 | Induced |
| VAMP3 | 9341 | Hs.66708 | Induced |
| GNS | 2799 | Hs.334534 | Induced |
| RPL7 | 6129 | Hs.571841 | Induced |
| PNPLA2 | 57104 | Hs.654697 | Induced |
| WIPI1 | 55062 | Hs.463964 | Induced |
| CIRBP | 1153 | Hs.634522 | Induced |
| KCTD11 | 147040 | Hs.592112 | Induced |
| INPP5A | 3632 | Hs.523360 | Induced |
| PREPL | 9581 | Hs.444349 | Induced |
| IRS1 | 3667 | Hs.471508 | Induced |
| KPNA3 | 3839 | Hs.527919 | Induced |
| DYNC1I2 | 1781 | Hs.546250 | Induced |
| CETN2 | 1069 | Hs.82794 | Induced |
| C1orf128 | 57095 | Hs.31819 | Induced |
| CRABP2 | 1382 | Hs.405662 | Induced |
| EST_AA399253 | | | Induced |
| C20orf11 | 54994 | Hs.353013 | Induced |
| ICMT | 23463 | Hs.515688 | Induced |
| CHMP2B | 25978 | Hs.476930 | Induced |
| DNAJB6 | 10049 | Hs.490745 | Induced |
| FAM62A | 23344 | Hs.632729 | Induced |
| RSNL2 | 79745 | Hs.122927 | Induced |
| GORASP2 | 26003 | Hs.431317 | Induced |
| LOC339984 | 339984 | Hs.592482 | Induced |
| C1orf24 | 116496 | Hs.518662 | Induced |
| COL15A1 | 1306 | Hs.409034 | Induced |
| LOC286058 | 286058 | Hs.638582 | Induced |
| SRPK1 | 6732 | Hs.443861 | Induced |
| TGFB1I1 | 7041 | Hs.513530 | Induced |
| ANXA9 | 8416 | Hs.653223 | Induced |
| CFHR1 | 3078 | Hs.575869 | Induced |
| HBP1 | 26959 | Hs.162032 | Induced |
| DGAT1 | 8694 | Hs.521954 | Induced |
| ALDH9A1 | 223 | Hs.2533 | Induced |
| LTF | 4057 | Hs.529517 | Induced |
| GALNTL1 | 57452 | Hs.21035 | Induced |
| ELTD1 | 64123 | Hs.132314 | Induced |
| AQP1 | 358 | Hs.76152 | Induced |
| RAB3IL1 | 5866 | Hs.13759 | Induced |
| SMOC2 | 64094 | Hs.487200 | Induced |
| ABCA1 | 19 | Hs.429294 | Induced |
| SLC44A1 | 23446 | Hs.573495 | Induced |
| SYNE1 | 23345 | Hs.12967 | Induced |
| DKFZP564B147 | 26071 | Hs.460924 | Induced |
| TM9SF1 | 10548 | Hs.91586 | Induced |
| GBE1 | 2632 | Hs.436062 | Induced |
| LOC286170 | 286170 | Hs.370312 | Induced |
| LOC619208 | 619208 | | Induced |
| FOXC1 | 2296 | Hs.348883 | Induced |
| SLC24A3 | 57419 | Hs.654790 | Induced |
| REV3L | 5980 | Hs.232021 | Induced |
| PRDM2 | 7799 | Hs.371823 | Induced |
| EVI5 | 7813 | Hs.656836 | Induced |
| MYST3 | 7994 | Hs.491577 | Induced |
| STEAP1 | 26872 | Hs.61635 | Induced |
| EPB41L1 | 2036 | Hs.437422 | Induced |
| PPP1R3C | 5507 | Hs.303090 | Induced |
| MAP1A | 4130 | Hs.194301 | Induced |
| ABLIM3 | 22885 | Hs.49688 | Induced |
| HINT3 | 135114 | Hs.72325 | Induced |
| EML1 | 2009 | Hs.12451 | Induced |
| CORO2B | 10391 | Hs.551213 | Induced |
| MYH10 | 4628 | Hs.16355 | Induced |
| DOC1 | 11259 | | Induced |
| GAMT | 2593 | Hs.81131 | Induced |
| PLEKHA5 | 54477 | Hs.188614 | Induced |
| GLIS2 | 84662 | Hs.592087 | Induced |
| EBF | 1879 | Hs.657753 | Induced |
| CCDC109A | 90550 | Hs.591366 | Induced |
| YME1L1 | 10730 | Hs.499145 | Induced |
| SORBS1 | 10580 | Hs.38621 | Induced |
| SDCCAG8 | 10806 | Hs.591530 | Induced |
| GFM1 | 85476 | Hs.518355 | Induced |
| COX6A1 | 1337 | Hs.497118 | Induced |
| TSR2 | 90121 | Hs.522662 | Induced |
| PPP2R5A | 5525 | Hs.497684 | Induced |
| C4orf14 | 84273 | Hs.8715 | Induced |
| EST_AA424653 | | | Induced |
| C20orf7 | 79133 | Hs.472165 | Induced |
| SMC3 | 9126 | Hs.24485 | Induced |
| SGPL1 | 8879 | Hs.499984 | Induced |
| GPR124 | 25960 | Hs.274136 | Induced |
| GPR157 | 80045 | Hs.31181 | Induced |
| KBTBD11 | 9920 | Hs.5333 | Induced |
| FKBP9 | 11328 | Hs.103934 | Induced |
| KLF10 | 7071 | Hs.435001 | Induced |
| GNAI3 | 2773 | Hs.73799 | Induced |
| MEGF9 | 1955 | Hs.494977 | Induced |
| SMARCA2 | 6595 | Hs.298990 | Induced |
| TFF3 | 7033 | Hs.82961 | Induced |
| NR2F6 | 2063 | Hs.466148 | Induced |
| SVEP1 | 79987 | Hs.522334 | Induced |
| PTRH1 | 138428 | Hs.643598 | Induced |
| ACLY | 47 | Hs.387567 | Induced |
| KLB | 152831 | Hs.90756 | Induced |
| TMEM131 | 23505 | Hs.469376 | Induced |
| PDE4B | 5142 | Hs.198072 | Induced |
| ANGPTL2 | 23452 | Hs.653262 | Induced |
| SREBF1 | 6720 | Hs.592123 | Induced |
| KHDRBS3 | 10656 | Hs.444558 | Induced |
| EST_AA620591 | | | Induced |
| ERG | 2078 | Hs.473819 | Induced |
| SFRP2 | 6423 | Hs.481022 | Induced |
| CALU | 813 | Hs.643549 | Induced |
| MPP7 | 143098 | Hs.499159 | Induced |
| USMG5 | 84833 | Hs.500921 | Induced |
| MAP2K3 | 5606 | Hs.514012 | Induced |
| TMEM119 | 338773 | Hs.449718 | Induced |
| MYCL1 | 4610 | Hs.437922 | Induced |
| DEGS1 | 8560 | Hs.299878 | Induced |
| MANSC1 | 54682 | Hs.591145 | Induced |
| KLF5 | 688 | Hs.508234 | Induced |
| NOL3 | 8996 | Hs.513667 | Induced |
| MLLT4 | 4301 | Hs.644024 | Induced |
| PHYHD1 | 254295 | Hs.308340 | Induced |
| INADL | 10207 | Hs.478125 | Induced |
| mtRNA_ND2 | | | Induced |
| UBE2M | 9040 | Hs.406068 | Induced |
| ZAK | 51776 | Hs.444451 | Induced |
| EREG | 2069 | Hs.115263 | Induced |
| Gcom1 | 145781 | Hs.437256 | Induced |
| NES | 10763 | Hs.527971 | Induced |
| LIN7B | 64130 | Hs.221737 | Induced |
| ATP2B4 | 493 | Hs.343522 | Induced |
| XM_496099 | 400470 | | Induced |
| EST_AA495812 | | | Induced |
| SSFA2 | 6744 | Hs.591602 | Induced |
| CYTB | 4519 | | Induced |
| PLAGL1 | 5325 | Hs.444975 | Induced |
| ADIPOR2 | 79602 | Hs.371642 | Induced |
| GPR146 | 115330 | Hs.585007 | Induced |
| MYLK | 4638 | Hs.556600 | Induced |
| FAM80B | 57494 | Hs.504670 | Induced |
| ARHGEF7 | 8874 | Hs.508738 | Induced |
| CAV2 | 858 | Hs.212332 | Induced |
| PLIN | 5346 | Hs.103253 | Induced |
| ST7OT1 | 93653 | Hs.597516 | Induced |
| ZNF407 | 55628 | Hs.536490 | Induced |
| MPDZ | 8777 | Hs.169378 | Induced |
| ZDHHC23 | 254887 | Hs.21902 | Induced |
| EST_AA291159 | | | Induced |
| WFS1 | 7466 | Hs.518602 | Induced |
| RAB5C | 5878 | Hs.127764 | Induced |
| ACTA2 | 59 | Hs.500483 | Induced |
| ARF6 | 382 | Hs.525330 | Induced |
| DDAH1 | 23576 | Hs.379858 | Induced |
| ATP2A2 | 488 | Hs.506759 | Induced |
| POR | 5447 | Hs.354056 | Induced |
| DMKN | 93099 | Hs.417795 | Induced |
| JAM3 | 83700 | Hs.150718 | Induced |
| RBMS1 | 5937 | Hs.470412 | Induced |
| BMP4 | 652 | Hs.68879 | Induced |
| GSTA4 | 2941 | Hs.485557 | Induced |
| TIMM8B | 26521 | Hs.279915 | Induced |
| CSNK2A2 | 1459 | Hs.82201 | Induced |
| mtRNA_ND4L | | | Induced |
| MKL2 | 57496 | Hs.592047 | Induced |
| PPP2R3A | 5523 | Hs.518155 | Induced |
| CDH11 | 1009 | Hs.116471 | Induced |
| QKI | 9444 | Hs.510324 | Induced |
| KDELC2 | 143888 | Hs.83286 | Induced |
| RTN3 | 10313 | Hs.473761 | Induced |
| LHFP | 10186 | Hs.507798 | Induced |
| ENPP2 | 5168 | Hs.190977 | Induced |
| SLC29A4 | 222962 | Hs.4302 | Induced |
| CHRDL1 | 91851 | Hs.496587 | Induced |
| DDEF2 | 8853 | Hs.555902 | Induced |
| ITSN1 | 6453 | Hs.160324 | Induced |
| ALDH1A3 | 220 | Hs.459538 | Induced |
| SDCCAG10 | 10283 | Hs.371372 | Induced |
| WDR47 | 22911 | Hs.654760 | Induced |
| ITGB1BP1 | 9270 | Hs.467662 | Induced |
| GNAI1 | 2770 | Hs.134587 | Induced |
| MEGF9 | 1955 | Hs.494977 | Induced |
| PXDN | 7837 | Hs.332197 | Induced |
| C12orf47 | 51275 | Hs.333120 | Induced |
| FLJ14834 | 84935 | Hs.616329 | Induced |
| SBEM | 118430 | Hs.348419 | Induced |
| RPL3 | 6122 | Hs.119598 | Induced |
| HSPA12A | 259217 | Hs.654682 | Induced |
| P2RY14 | 9934 | Hs.2465 | Induced |
| WWTR1 | 25937 | Hs.477921 | Induced |
| MSH3 | 4437 | Hs.280987 | Induced |

### Example 5

### Refining the PDGFR, Kit and Abl TKI Responsive Signature

The PDGFR, Kit, and Abl TKI Responsive Signature described in Example 4 and Table 2 was further refined using statistical parameters to identify a TKI Responsive Signatures comprising 49 genes (Table 3). The genetic sequences set forth in Table 2 and Example 4 were shown to be altered in the autoimmune disease tissue (SSc skin) following exposure to the TKI imatinib. A useful response profile may be obtained from all or a part of the gene dataset, usually the TKI Responsive Signature will comprise information from at least about 5 genes, more usually at least about 10 genes, at least about 15 genes, at least about 20 genes, at least about 25 genes, at least about 30, at least about 35, at least about 40, or more, up to the complete dataset. Where a subset of the dataset is used, the subset may comprise induced genes, repressed genes, or a combination thereof. The microarray analysis results presented in Table 2 were further used to identify genes with two-fold reduced and two-fold elevated expression in SSc skin biopsy samples obtained pre-treatment as compared to 1+ months post-treatment. The performance characteristics of 1050, 102, 49 and 10 gene TKI Responsive Signatures are presented in Table 4.

TABLE 3

| | | Expression pattern |
|---|---|---|
| Gene Symbol | Locus Link | before Imatinib treatment |
| CDC20 | 991 | Induced |
| HBA1 | 3039 | Induced |
| SFRS7 | 6432 | Induced |
| EST_AI791445 | 28566 | Induced |
| KIAA1794 | 55215 | Induced |
| CLK1 | 1195 | Induced |
| HBA2 | 3040 | Induced |
| MKI67 | 4288 | Induced |
| CCNB2 | 9133 | Induced |
| ARL4C | 10123 | Induced |
| NOL5A | 10528 | Induced |
| UBE2C | 11065 | Induced |
| NUSAP1 | 51203 | Induced |
| CTA-246H3.1 | 91353 | Induced |
| TBC1D10C | 374403 | Induced |
| PDK4 | 5166 | Repressed |
| DEGS1 | 8560 | Repressed |
| KLF4 | 9314 | Repressed |
| PSORS1C2 | 170680 | Repressed |
| LYPD5 | 284348 | Repressed |
| SERPING1 | 710 | Repressed |
| CALM1 | 801 | Repressed |
| CD1A | 909 | Repressed |
| COL5A2 | 1290 | Repressed |
| COL12A1 | 1303 | Repressed |
| CTGF | 1490 | Repressed |
| ERG | 2078 | Repressed |
| FBLN1 | 2192 | Repressed |
| GATM | 2628 | Repressed |
| H3F3B | 3021 | Repressed |
| ID4 | 3400 | Repressed |
| PER1 | 5187 | Repressed |
| SFRP2 | 6423 | Repressed |
| SLC2A1 | 6513 | Repressed |
| SULT2B1 | 6820 | Repressed |
| THBS1 | 7057 | Repressed |
| IL1R2 | 7850 | Repressed |
| SORBS1 | 10580 | Repressed |
| ENDOD1 | 23052 | Repressed |
| ANKRD15 | 23189 | Repressed |
| SEC15L2 | 23233 | Repressed |
| RAI14 | 26064 | Repressed |
| ELMOD1 | 55531 | Repressed |
| SVEP1 | 79987 | Repressed |
| CGNL1 | 84952 | Repressed |
| MPP7 | 143098 | Repressed |
| LOC143381 | 143381 | Repressed |
| LCE5A | 254910 | Repressed |
| LOC342897 | 342897 | Repressed |

### Example 6

### Identification of a PDGFR, Kit and Abl TKI Responsive Signatures in Other Autoimmune or Inflammatory Diseases

The refined 49 gene PDGFR, Kit, and Abl TKI Responsive Signature identified in Example 5 can be used to interrogate gene expression datasets from a variety of diseases and individual patients to identify specific diseases and individual patients likely to respond to therapy with a PDGFR, Kit, and Abl TKI.

The 49 gene PDGFR, Kit and Abl TKI Responsive Signature in Table 3 was compared against gene expression analyses from independent patient populations (referred to as the patient datasets), including datasets obtained from autoimmune or other inflammatory disease targeted tissues. These datasets are deposited in and publicly available from the NCBI's Gene Expression Omnibus (GEO). The gene expression datasets were obtained for RA, Crohn's/Colitis, and IPF, and hierarchical clustering was carried out to determine if the 49 gene PDGFR, Kit, and Abl TKI Responsive Signature (Table 3) is present in these diseases. Using hierarchical clustering, selected patients with Rheumatoid arthritis, Crohn's/Colitis, and Idiopathic pulmonary fibrosis were identified as possessing the PDGFR, Kit, and Abl TKI Responsive Signature (FIG. 5).

### Example 7

### Identification of Core PDGFR-Abl-Kit and PDGFR-Abl-Kit-Fms TKI Responsive Signatures

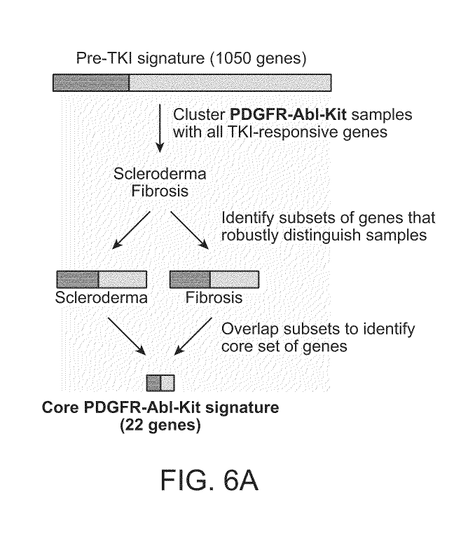

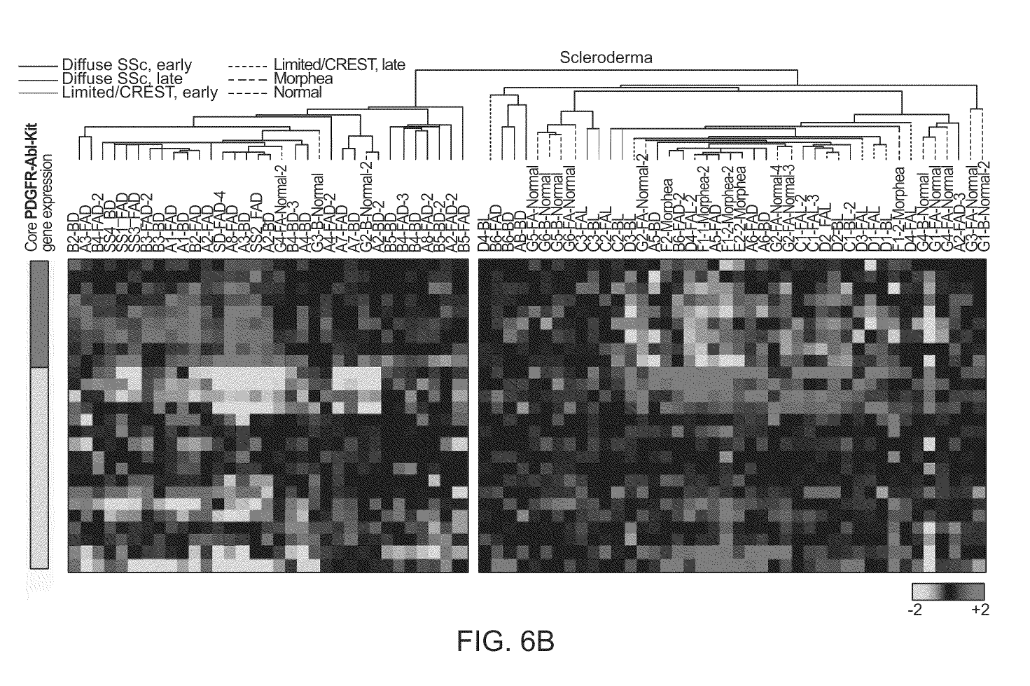

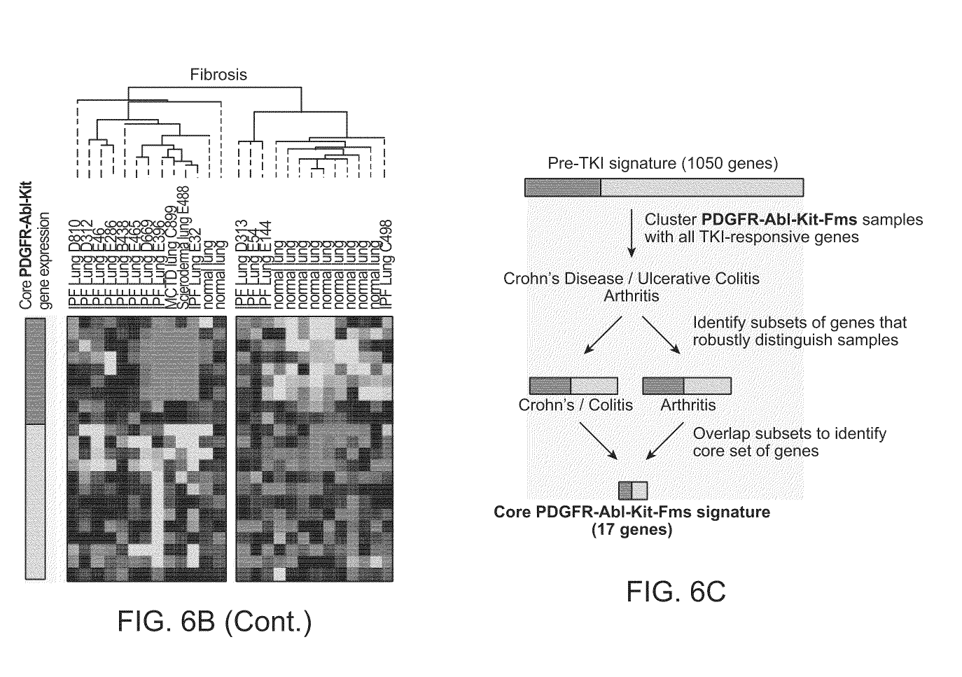

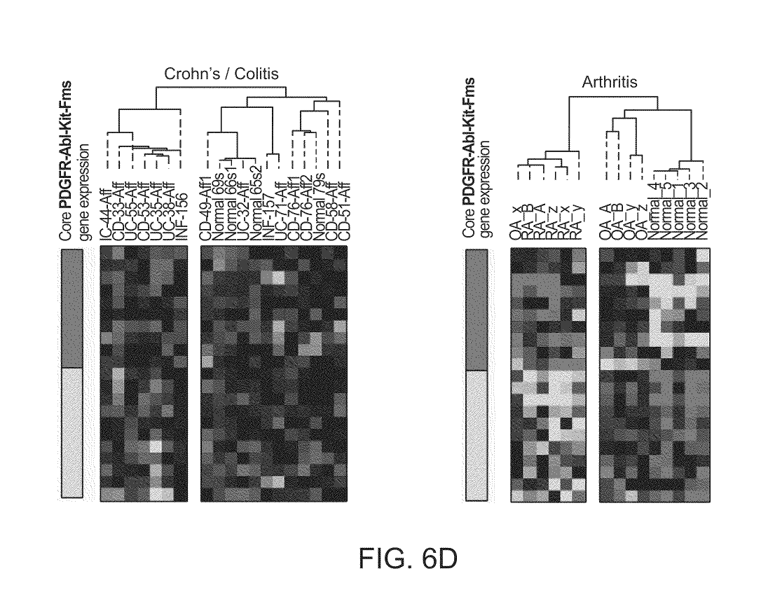

To identify core gene signatures that distinguish autoimmune diseases driven by the PDGFR, Abl, and Kit tyrosine kinases, Scleroderma (Milano et al., PLoS ONE, 2008) and Idiopathic pulmonary fibrosis (Pardo et al., PLoS Med, 2005) samples were clustered with all 1050 genes comprising the TKI Responsive Signature (Table 2). Genes that robustly distinguish the disease samples from normal controls were identified for each disease type, and the overlap between the two gene lists formed a core PDGFR-Abl-Kit gene signature composed of 22 genes (FIG. 6 A) (Table 7). Seventy-five gene expression profiles of Scleroderma samples and 26 gene expression profiles of Fibrosis samples were analyzed by unsupervised hierarchical clustering of the 22 PDGFR-Abl-Kit signature genes (FIG. 6 B).

To identify genes that distinguish autoimmune diseases driven by the PDGFR, Abl, Kit, and Fms tyrosine kinases, Crohn's disease and Ulcerative colitis (Wu et al., Inflamm Bowel Dis, 2007) as well as Rheumatoid arthritis and Osteoarthritis (Lorenz et al., Proteomics, 2003) samples were clustered with all 1050 genes comprising the TKI Responsive Signature. Genes that robustly distinguish the disease samples and normal controls were identified for each disease type, and the overlap between the two gene lists formed a core PDGFR-Abl-Kit-Fms Responsive Signature comprising 17 genes (FIG. 6 C) (Table 8). Nineteen gene expression profiles of Crohn's disease and Ulcerative colitis samples, and 15 gene expression profiles of Rheumatoid arthritis and Osteoarthritis samples, were analyzed by unsupervised hierarchical clustering of the 17 genes comprising the PDGFR-Abl-Kit-Fms Responsive Signature (FIG. 6 D). The performance characteristics of these TKI Responsive Gene Signatures are detailed in Table 4.

TABLE 4
Performance of TKI Responsive Gene Signatures.

| | | | | | Score (0-3, see below for | | | | | |
|---|---|---|---|---|---|---|---|---|---|---|
| | | | | | explanation of scores) | | | | | |
| | | | | | Orig. | | SET2 | SET3 | Core | Core |
| | | Samples being | # of | Receptors | (1050 | SET1 (102 | (49 | (10 | PDGFR-Abl- | PDGFR-Abl- |
| Disease | Author | clustered & compared | samples | Involved | genes) | genes) | genes) | genes) | Kit (22 genes) | Kit-Fms (17 genes) |
| Scleroderma | Milano | Diffuse scleroderma vs. | 75 | PDGFR- | 3 | 3 | 3 | 2 | 3 | 3 |
| | | normal/CREST/morphea | | Abl-Kit | | | | | | |
| Idiopathic | Pardo | IPF vs. normal | 26 | PDGFR- | 3 | 3 | 2 | 2 | 3 | 1 |
| Pulmonary | | | | Abl-Kit | | | | | | |
| Fibrosis | | | | | | | | | | |
| Crohn's | Wu | CD & UC vs. normal | 19 | PDGFR- | 3 | 2 | 3 | 1 | 0 | 3 |
| Disease, | | | | Abl-Kit- | | | | | | |
| Ulcerative | | | | Fms | | | | | | |
| Colitis | | | | | | | | | | |
| Rheumatoid- | Lorenz | RA & OA vs. normal | 15 | PDGFR- | 3 | 3 | 3 | 3 | 3 | 3 |
| & Osteo- | | | | Abl-Kit- | | | | | | |
| Arthritis | | | | Fms | | | | | | |

| | |
|---|---|
| Scoring | |
| key: | |
| 0 - Poor | A majority of patients with the disease did not posses the |
| | TKI Responsive Signature |
| 1 - Fair | Approximately 50% of the samples from patients with the |
| | disease who are likely to respond have the TKI Responsive |
| | Signature compared to patients with another disease or |
| | people without disease (normals) |
| 2 - Good | Approximately 75% of the samples from patients with the |
| | disease who are likely to respond have the TKI Responsive |
| | Signature compared to patients with another disease or |
| | people without disease (normals) |
| 3 - Excellent | Approximately 90% of the samples from patients with the |
| | disease who are likely to respond have the TKI Responsive |
| | Signature compared to patients with another disease or |
| | people without disease (normals) |

TABLE 5
10 gene TKI Responsive Signature

| | | Expression pattern |
|---|---|---|
| Gene Symbol | Locus Link | before Imatinib treatment |
| CDC20 | 991 | Induced |
| HBA1 | 3039 | Induced |
| SFRS7 | 6432 | Induced |
| EST_AI791445 | 28566 | Induced |
| KIAA1794 | 55215 | Induced |
| PDK4 | 5166 | Repressed |
| DEGS1 | 8560 | Repressed |
| KLF4 | 9314 | Repressed |
| PSORS1C2 | 170680 | Repressed |
| LYPD5 | 284348 | Repressed |

TABLE 6
102 gene TKI Responsive Signature

| | | Expression pattern |
|---|---|---|
| Gene Symbol | Locus Link | before Imatinib treatment |
| CCNB1 | 891 | Induced |
| CDC20 | 991 | Induced |
| CKS2 | 1164 | Induced |
| CLK1 | 1195 | Induced |
| EZH2 | 2146 | Induced |
| FKBP5 | 2289 | Induced |
| FUS | 2521 | Induced |
| HBA1 | 3039 | Induced |
| HBA2 | 3040 | Induced |
| MCM5 | 4174 | Induced |
| MKI67 | 4288 | Induced |
| MT1F | 4494 | Induced |
| SFRS7 | 6432 | Induced |
| CCNB2 | 9133 | Induced |
| SDCCAG1 | 9147 | Induced |
| ARL4C | 10123 | Induced |
| NOL5A | 10528 | Induced |
| UBE2C | 11065 | Induced |
| NUP210 | 23225 | Induced |
| EST_AI791445 | 28566 | Induced |
| NUSAP1 | 51203 | Induced |
| KIAA1794 | 55215 | Induced |
| DDX55 | 57696 | Induced |
| ATF7IP2 | 80063 | Induced |
| CTA-246H3.1 | 91353 | Induced |
| KIAA1245 | 149013 | Induced |
| TBC1D10C | 374403 | Induced |
| HSUP1 | 441951 | Induced |
| AGTRL1 | 187 | Repressed |
| APOD | 347 | Repressed |
| ATP2B4 | 493 | Repressed |
| SERPING1 | 710 | Repressed |
| CALM1 | 801 | Repressed |
| CD1A | 909 | Repressed |
| COL1A1 | 1277 | Repressed |
| COL1A2 | 1278 | Repressed |
| COL5A1 | 1289 | Repressed |
| COL5A2 | 1290 | Repressed |
| COL12A1 | 1303 | Repressed |
| CTGF | 1490 | Repressed |
| DBN1 | 1627 | Repressed |
| EBF | 1879 | Repressed |
| ERG | 2078 | Repressed |
| FBLN1 | 2192 | Repressed |
| GALNT1 | 2589 | Repressed |
| GATM | 2628 | Repressed |
| GJA1 | 2697 | Repressed |
| H3F3B | 3021 | Repressed |
| ID4 | 3400 | Repressed |
| IGFBP3 | 3486 | Repressed |
| MYCL1 | 4610 | Repressed |
| PDE2A | 5138 | Repressed |
| PDGFRB | 5159 | Repressed |
| PDK4 | 5166 | Repressed |
| PER1 | 5187 | Repressed |
| PTGDS | 5730 | Repressed |
| SFRP2 | 6423 | Repressed |
| SLC2A1 | 6513 | Repressed |
| SULT2B1 | 6820 | Repressed |
| THBS1 | 7057 | Repressed |
| TIE1 | 7075 | Repressed |
| IL1R2 | 7850 | Repressed |
| DEGS1 | 8560 | Repressed |
| FCGBP | 8857 | Repressed |
| KLF4 | 9314 | Repressed |
| SORBS1 | 10580 | Repressed |
| NES | 10763 | Repressed |
| NCKAP1 | 10787 | Repressed |
| ENDOD1 | 23052 | Repressed |
| ANKRD15 | 23189 | Repressed |
| SEC15L2 | 23233 | Repressed |
| SASH1 | 23328 | Repressed |
| KRT23 | 25984 | Repressed |
| RAI14 | 26064 | Repressed |
| KIF26A | 26153 | Repressed |
| RASD1 | 51655 | Repressed |
| SOX18 | 54345 | Repressed |
| ELMOD1 | 55531 | Repressed |
| POF1B | 79983 | Repressed |
| SVEP1 | 79987 | Repressed |
| C9orf58 | 83543 | Repressed |
| B3GNT5 | 84002 | Repressed |
| EBPL | 84650 | Repressed |
| CGNL1 | 84952 | Repressed |
| TMEM88 | 92162 | Repressed |
| CHMP4C | 92421 | Repressed |
| MAL2 | 114569 | Repressed |
| GPR146 | 115330 | Repressed |
| CTHRC1 | 115908 | Repressed |
| HSPB6 | 126393 | Repressed |
| SPTLC2L | 140911 | Repressed |
| MPP7 | 143098 | Repressed |
| LOC143381 | 143381 | Repressed |
| SNF1LK | 150094 | Repressed |
| PSORS1C2 | 170680 | Repressed |
| MSRB3 | 253827 | Repressed |
| LCE5A | 254910 | Repressed |
| LYPD5 | 284348 | Repressed |
| LOC286170 | 286170 | Repressed |
| TMEM119 | 338773 | Repressed |
| LOC342897 | 342897 | Repressed |
| LOC441158 | 441158 | Repressed |

TABLE 7
22 gene core PDGFR-Abl-Kit Signature

| | | Expression pattern |
|---|---|---|
| Gene Symbol | Locus Link | before Imatinib treatment |
| CCNB1 | 891 | Induced |
| CKS2 | 1164 | Induced |
| KIF11 | 3832 | Induced |
| LIG1 | 3978 | Induced |
| KIF20A | 10112 | Induced |
| UBE2C | 11065 | Induced |
| UBE2T | 29089 | Induced |
| NUSAP1 | 51203 | Induced |
| CDCA8 | 55143 | Induced |
| ARF6 | 382 | Repressed |
| BCL2L2 | 599 | Repressed |
| SERPING1 | 710 | Repressed |
| CAV1 | 857 | Repressed |
| CLDN5 | 7122 | Repressed |
| RGS5 | 8490 | Repressed |
| RAI2 | 10742 | Repressed |
| C10orf116 | 10974 | Repressed |
| CYFIP1 | 23191 | Repressed |
| CXCR7 | 57007 | Repressed |
| C1orf128 | 57095 | Repressed |
| CGNL1 | 84952 | Repressed |
| FAM129A | 116496 | Repressed |

TABLE 8
17 gene core PDGFR-Abl-Kit-Fms Signature

| | | Expression pattern |
|---|---|---|
| Gene Symbol | Locus Link | before Imatinib treatment |
| CDC20 | 991 | Induced |
| SLC37A4 | 2542 | Induced |
| IGLL1 | 3543 | Induced |
| TK1 | 7083 | Induced |
| TYMS | 7298 | Induced |
| ARL4C | 10123 | Induced |
| NOL5A | 10528 | Induced |
| AQP7 | 364 | Repressed |
| GBE1 | 2632 | Repressed |
| PDE2A | 5138 | Repressed |
| TGFA | 7039 | Repressed |
| TGFBR3 | 7049 | Repressed |
| CSDA | 8531 | Repressed |
| AOC3 | 8639 | Repressed |
| KHDRBS3 | 10656 | Repressed |
| KANK1 | 23189 | Repressed |
| ADIPOR2 | 79602 | Repressed |

### Example 8

### Independent Identification of a PDGFR, Abl, Kit and Fms TKI Responsive Signatures in Rheumatoid Arthritis

A PDGFR, Abl, Kit, and Fms TKI Responsive Signature is identified in rheumatoid arthritis by performing gene expression analysis on synovial biopsies obtained pre- and post-treatment with a PDGFR, Abl, Kit, and Fms TKI. Prior to treatment, a needle is used to obtain synovial fluid containing inflammatory cells or a trochar system is used to obtain a biopsy of the synovial lining from inflamed knees or other joints in patients with RA. These patients are then treated with a PDGFR, Abl, Kit, and Fms TKI, and their responses to therapy assessed based on Disease Activity Scores (DAS) and American College of Rheumatology Response Scores (ACR Response) at baseline and following 3 or 6 months of treatment. At 3 or 6 months of treatment, a repeat synovial fluid sample or trochar system synovial biopsy is obtained. RNA is isolated from both the pre-treatment and post-treatment synovial fluid or biopsy samples, and DNA array analysis is performed to determine the gene expression profiles. Statistical algorithms are applied to identify a gene profile associated with a positive clinical response to the PDGFR, Kit, Abl, and Fms TKI. Hierarchical clustering and Pearson correlation analysis are performed to determine the specific RA patients, as well as samples derived from patients with other autoimmune or other inflammatory diseases, that possess the PDGFR, Kit, Abl, and Fms TKI response profile.

### Example 7

### Independent Identification of a PDGFR, Abl, and Kit TKI Responsive Signatures in Graft-Versus-Host-Disease

The PDGFR, Abl, Kit and Fms TKI Responsive Signature is identified in graft-versus-host-disease (GVHD) by performing gene expression analysis on skin, gastrointestinal tract, liver, or other tissue biopsies obtained pre- and post-treatment with a PDGFR, Abl, and Kit TKI. Prior to treatment, a needle is used to obtain synovial fluid containing inflammatory cells or a trochar system is used to obtain a biopsy of an inflamed tissue in a patient with GVHD. These patients are then treated with a PDGFR, Abl, and Kit TKI, and their responses to therapy assessed based on Disease Activity Scores (DAS) and American College of Rheumatology Response Scores (ACR Response) at baseline and following 3 or 6 months of treatment. At 3 or 6 months of treatment, a repeat biopsy is obtained. RNA is isolated from both the pre-treatment and post-treatment synovial fluid or biopsy samples, and DNA array analysis is performed to determine the gene expression profiles. Statistical algorithms are applied to identify a GVHD Responsive Signature based on its statistical association with a positive clinical response to the PDGFR, Abl, and Kit TKI. Hierarchical clustering and Pearson correlation analysis are performed to determine the specific GVHD patients, as well as samples derived from patients with other autoimmune or other inflammatory diseases, that possess the PDGFR, Abl, and Kit TKI response profile.

### Example 8

### Identification of TKI Responsive Signatures in Autoimmune or Other Inflammatory Diseases

The TKI Responsive Signatures in Examples 6 and 7 can be further refined and used to identify individual patients with autoimmune and other inflammatory diseases likely to respond to TKI therapy. To identify other TKI responsive diseases, the TKI Responsive Signatures identified in Example 6 or 7 are compared against gene expression analyses from independent patient populations (referred to as the patient datasets), including datasets obtained from autoimmune or other inflammatory disease targeted tissues. These datasets are deposited in and publicly available from the NCBI's Gene Expression Omnibus (GEO). The gene expression datasets from a wide variety of autoimmune or other inflammatory diseases including Crohn's, IPF, psoriasis, multiple sclerosis, primary biliary cirrhosis, autoimmune hepatitis, and other autoimmune or other inflammatory diseases are obtained. Hierarchical clustering is performed to determine if the PDGFR, Abl, Kit, and Fms or PDGFR, Abl, and Kit TKI Responsive Signatures are present in these diseases. The approach is demonstrated in FIGS. 5 and 6, and summarized in Table 4.

### Example 9

### Identification of a PDGFR, Kit, Fms, and Abl TKI Responsive Signature in Autoimmune or Other Inflammatory Diseases

Certain autoimmune or other inflammatory diseases possess diverse tyrosine kinases and cellular responses contributing to pathogenesis, and as a result exhibit TKI Responsive Signatures that encompass both the PDGFR, Kit, and Abl signature as well as the PDGFR, Abl, Kit, and Fms signature. Examples of such diseases include rheumatoid arthritis and Crohn's disease. Both diseases are characterized by excessive fibroblast proliferation, in part mediated by PDGFR and Abl, which results in the formation of pannus tissue that invades cartilage and bone in RA as well as the formation of strictures which causes bowel dysfunction in Crohn's. Both RA and Crohn's also exhibit infiltration of mast cells, and activation of mast cells by Kit results in release of pro-inflammatory mediators and degradative enzymes. Further, Fms-mediated macrophage production of TNF-alpha plays a central role in the pathogenesis of RA and Crohn's. Thus, RA, Crohn's and certain other inflammatory diseases are expected to exhibit TKI Response Signatures that include genes in both the PDGFR, Kit, and Abl signature as well as the PDGFR, Abl, Kit, and Fms signature.

### Example 10

### Use of the TKI Responsive Signature to Identify Individual Systemic Sclerosis Patients Likely to Respond to PDGFR, Kit, and Abl TKI Therapy

Individual patients with SSc or possible SSc undergo skin biopsy of the forearm, and DNA microarray analysis is performed to determine the individual patients' gene expression profile. The individual patients' gene expression profile is then compared with the TKI Responsive Signature to predict whether the patient will respond to PDGFR, Kit, and Abl TKI therapy. Based on the comparison, the individual is determined to be low-responsive or non-responsive to TKI treatment, or likely to be responsive, or responsive to TKI treatment. Based on the predicted response, the physician determines whether to treat the individual patient with the TKI, or to not treat the patient with a TKI.

### Example 11

### Use of the TKI Responsive Signature to Identify Individuals with Other Autoimmune or Inflammatory Diseases Likely to Respond to TKI Therapy

Individual patients with autoimmune or other inflammatory diseases known to possess a TKI Response Signature, as well as patients with poorly defined inflammatory processes (such as lung or liver inflammation), can be further characterized for likelihood to respond to TKI therapy using the TKI Responsive Signatures. The individual with an autoimmune or other inflammatory disease undergoes biopsy of the tissue (or cells) involved in the inflammatory process, RNA is extracted from the biopsied tissue or cells, and DNA array analysis is performed to determine the patient's TKI Responsive Signature profile. The individual patient's TKI Responsive Signature profile is then statistically compared with the PDGFR, Kit, and Abl TKI Responsive Signature and the PDGFR, Abl, Kit, and Fms TKI Responsive Signature to determine the best match. Based on the best match of the patient's TKI Responsive Signature profile, the corresponding TKI is selected to treat the individual patient. The physician then prescribes the selected TKI for the patient.

### Example 12

### Use of the TKI Responsive Signature to Select Patients for Enrollment in Human Clinical Trials

A TKI Responsive Signature can be used to streamline clinical trials in SSc and/or other autoimmune or inflammatory diseases. For SSc, the Phase II and/or Phase III trial is designed to enroll patients based on: (i) meeting the diagnostic criteria for SSc, (ii) failing conventional immunomodulatory drug therapy, and (iii) possessing the PDGFR, Kit, and Abl TKI Responsive Signature. After the patient undergoes initial screening based on the diagnostic criteria for SSc and having failed therapy with other drugs, a skin biopsy is obtained, RNA isolated, DNA array analysis performed, and the individual patient's TKI Responsive Signature profile determined. The patients TKI Responsive Signature profile is then statistically matched to the PDGFR, Kit, and Abl TKI Responsive Signatures presented in Tables 2 and 3. If the patient's TKI Responsive Signature profile sufficiently matches the PDGFR, Kit, and Abl signature, then the patient is enrolled in the Phase II or Phase III trial.

### Example 13

### Use of the TKI Responsive Signature as a Pharmacodynamic Marker in Human Clinical Trials

A major challenge in drug development is identifying the correct dose and obtaining early insights into whether a drug is exhibiting efficacy. In addition to pre-selecting patients likely to respond to TKI therapy (Example 12), the TKI Responsive Signatures can also be applied as pharmacodynamic (PD) markers in TKI development. Specifically, it is demonstrated that the TKI Responsive Signature normalizes following initiation of effective TKI therapy, with genes that are over-expressed in SSc exhibiting a decrease in expression towards expression levels present in normal skin while genes that are under-expressed in SSc exhibit an increase in expression towards expression levels present in normal skin. Thus, by obtaining serial tissue biopsies in SSc or other human trials the TKI Responsive Signature can be serially followed to determine if the TKI treated patients are responding to TKI therapy or not. This information can facilitate selection of an effective dose, and can be used for early identification of TKI drug candidates likely to show efficacy in larger trials. Beyond human clinical trials, in clinical practice such PD response profiles can also be used to follow patients being treated with TKIs to determine if they are manifesting meaningful responses, or if they are not experiencing benefit from treatment with a particular TKI.

### Example 14

### Characterization of the Specificity of Small Molecule TKIs

Small molecule TKIs are characterized to determine the specific receptor tyrosine kinases they inhibit, and based on their inhibitory profile they can be utilized to treat autoimmune or other inflammatory diseases exhibiting activation of the corresponding receptor tyrosine kinases. The specific tyrosine kinases inhibited by a particular small molecule inhibitor are determined using (i) in vitro kinase assays, (ii) in vitro cellular response assays, and (iii) other kinase inhibitor profiling methodologies. In vitro kinase assays involve incubating the specific tyrosine kinase with its substrate in the presence of a range of concentrations of the TKI, and determining the concentration of the inhibitor necessary to inhibit phosphorylation of the substrate. In vitro cellular response assays involve stimulating cells with ligands that activate specific tyrosine kinases in the presence of a range of TKI concentrations, and determining the concentration of the TKI necessary to inhibit a cellular response (proliferation, cytokine production, etc). Based on the specific kinases inhibited by a particular TKI, the TKI can be classified as inhibiting PDGFR, Kit, and Abl; inhibiting PDGFR, Kit, and Fms; inhibiting PDGFR, Kit, Fms, and Abl; inhibiting these kinases plus Flt3; and/or inhibiting other kinases. The TKI Responsive Signature of the individual patient or the TKI Responsive Signature for a particular autoimmune or other inflammatory disease is then matched with TKIs that inhibit the involved kinases, to identify the specific TKI(s) most likely to provide benefit to a patient or a particular autoimmune disease or other inflammatory disease.

### Example 15

### Identification of Gene Signatures for Tyrosine Kinase-Mediated Cellular Responses that Contribute to the Pathogenesis of Autoimmune Disease or Other Inflammatory Disease

Gene signatures for small molecule TKIs are generated using in vitro cell-based assays.

Cell lines or primary cell cultures representing the cell type(s) mediating pathogenesis are used for such studies. The following are examples of cell lines or primary cell cultures that can be used: 1) synovial fibroblasts or other fibroblast lines which mediate pannus formation in rheumatoid arthritis, bowel strictures in Crohn's, formation of plaques in multiple sclerosis, and the fibrosis and hardening of the skin in SSc; 2) tissue macrophage or murine peritoneal macrophage which produce pro-inflammatory cytokines including TNFα which contributes to Crohn's, rheumatoid arthritis, psoriasis, psoriatic arthritis, ankylosing spondylitis, and other autoimmune and inflammatory diseases; 3) mast cell line which is thought to contribute to inflammation in rheumatoid arthritis, Crohn's disease, and other autoimmune and inflammatory diseases; or 4) hematopoietic multipotent progenitors (MPP) and common lymphoid progenitors (CLP) that express Flt3 (CD135) and give rise to B cells, T cells, and other immune cells that contribute to the pathogenesis of autoimmune and other inflammatory diseases. A TKI Responsive Signature can be obtained by: stimulating the selected cell lines or primary cells with disease-relevant stimuli (molecules or ligands present and involved in activating these cells to contribute to the disease process) in the absence or presence of a TKI; measuring the cellular responses using standard read-outs; detecting genes in the cells; and comparing the gene profiles in the pre-stimulated, post-stimulated, and TKI-treated cells. Examples of cellular response read-outs include: measurement of: fibroblast proliferation by 3H-thymidine incorporation or cytokine production by ELISA; macrophage TNFα and other cytokine production by ELISA analysis of culture supernatants; mast cell TNFα, IL-6, and other inflammatory mediator release by ELISA analysis of culture supernatants; and hematopoietic multipotent progenitor (MPP) and common lymphoid progenitor (CLP) (stimulated by Flt3-ligand) development and maturation into B, T, and other immune cells. Changes in cellular genes can be assessed using DNA microarray analysis performed on RNA isolated from the cell lines or primary cell cultures pre-stimulation, as well as post-stimulation in the presence or absence of a TKI. As described above, bioinformatic analysis is applied to identify TKI Responsive Signatures.

For example, rheumatoid synovial fibroblasts are stimulated with PDGF ligand, or TGFβ, or TNFα, or other stimuli, or a combination of these stimuli, in the absence or presence of a TKI, and the cellular gene expression is determined pre-stimulation, post-stimulation and then in the presence of the TKI. Bioinformatic analysis is applied to determine the gene profile associated with and predictive of the response to the TKI by: identifying the upregulated or down-regulated genes in response to the stimuli, and identifying the aberrantly upregulated or down-regulated genes that are altered by the TKI. Those genes comprise the TKI Gene Signature that is associated with and predictive of a response to the TKI for the selected cell type. For tissue macrophage and murine peritoneal macrhopage, examples of stimuli include LPS, M-CSF, IL-34, and anti-FcR antibodies. For mast cells, examples of stimuli include stem cell factor (SCF) and anti-FcR antibodies. For hematopoietic precursors and other immune cells, examples of stimuli include Flt3-ligand and anti-antigen receptor antibodies.

In most autoimmune and other inflammatory diseases, multiple different cell types contribute to pathogenesis. In order to generate a TKI Gene Signature for an autoimmune or other inflammatory disease, changes in cellular gene expression would be determined for the cells contributing to the pathogenesis of a disease. For example, in rheumatoid arthritis four cell types are involved: 1) fibroblasts contribute to the formation of invasive pannus; 2) macrophages produce TNFα and other cytokines; 3) mast cells produce TNFα and other inflammatory mediators; and 4) B cells produce cytokines and autoantibodies. In contrast, in SSc two cell types are involved: fibroblasts contribute to skin fibrosis and hardening (sclerosis), while macrophage TNFα production does not play a central role in pathogenesis. Based on the specific cellular responses contributing to pathogenesis in a particular disease, bioinformatically one constructs a TKI Gene Signature profile representative of the autoimmune or inflammatory disease that is a composite of cellular changes in the various cell types contributing to the disease that is then predictive of a response to the selected TKI. For example, in RA the TKI Gene Signature is bioinformatically constructed from and incorporates genes aberrantly expressed in fibroblasts, macrophages, mast cells, and B cells—reflecting aberrant activity of class III receptor tyrosine kinases including PDGFR (and Abl) (fibroblasts), Fms (macrophage), Kit (mast and B cells), and Flt3 or Abl (B cells). In contrast, in SSc, the TKI Gene Signature is based on genes dysregulated in expression in fibroblasts and other inflammatory cells—reflecting aberrant activity of the class III receptor tyrosine kinases PDGFR (and Abl) and Kit (mast and B cells). Based on the datasets generated from relevant cell lines and primary cells for a particular disease, bioinformatic analysis can be utilized to integrate and combine the gene expression profiles from the individual cell types to generate a TKI Responsive Gene Signature for that particular disease for a particular TKI. In the example of rheumatoid arthritis, the TKI Responsive Gene Signature would be bioinformatically generated from the gene expression profiles obtained from the pre- and post-stimulated, and plus/minus TKI treated fibroblasts, macrophage, mast cells, and B cells. For SSc, the TKI Responsive Gene Signature would be bioinformatically generated from the gene expression profiles obtained from the pre- and post-stimulated, and plus/minus TKI treated fibroblasts and possibly other inflammatory cells.

1. A method for determining TKI therapy responsiveness of a patient with an autoimmune disease or other inflammatory disease comprising:
(a) determining expression levels for at least a subset of genes from the TKI Responsive Signature in a biological sample of the patient; and,
(b) comparing the expression levels of at least the subset of genes in the tissue sample to a pre-determined TKI responsive expression profile.

2. The method of claim 1 including the additional step of classifying the patient from which the biological sample was obtained as responsive if the comparison in (b) is positively correlated.

3. A method for determining TKI therapy responsiveness of a patient afflicted with an autoimmune disease or other inflammatory disease, the method comprising:
(a) determining expression levels of one or more genes in a biological sample of the patient afflicted with an autoimmune disease or other inflammatory disease wherein the one or more gene(s) are selected from a TKI Responsive Signature;
(b) comparing the expression levels of the one or more gene(s) in the biological sample of the patient in (a) to the expression levels of the one or more gene(s) comprising the TKI Responsive Signature; and,
(c) classifying the patient afflicted with the autoimmune or other inflammatory disease to either a non-responsive or responsive group based on the comparison in (b).

4. The method of claim 1 wherein determining the expression levels of one or more genes selected from the TKI Responsive Signature is by determining gene transcription levels, mRNA levels, translation levels, or protein or polypeptide levels or activity, or a combination thereof.

5. The method of claim 4 wherein the protein or polypeptide is detected by immunohistochemical analysis on the biological sample using an antibody that binds to the protein or polypeptide.

6. The method of claim 4 wherein the protein or polypeptide is detected by ELISA assay using an antibody that specifically binds to the protein or polypeptide.

7. The method of claim 4 wherein the protein or polypeptide is detected using an antibody array comprising an antibody that specifically binds to the protein or polypeptide.

8. The method of claim 4 wherein the mRNA is detected using a polynucleotide array comprising polynucleotides that hybridize to the mRNA.

9. The method of claim 4 wherein the mRNA is detected using polymerase chain reaction comprising polynucleotide primers to amplify the mRNA.

10. The method of claim 1, wherein the group of genes include at least 3, 4, 5, 6, 7, 8, 9, 10, 15, 20, 25, 30, 35, or 40 genes.

11. The method of claim 1 wherein the TKI Responsive Signature comprises genes in Table 2, 3, 5, 6, 7, or 8.

12. The method of claim 1, wherein the group of genes is selected from TKI Responsive Signature listed in Table 7 and 8 and wherein the TKI therapy includes one or more inhibitors for PDGFR, Abl, and Kit.

13. An array comprising polynucleotides hybridizing to TKI Responsive Signature genes in Table 2, 3, 5, 6, 7, or 8.

14. The method of claim 1 wherein the autoimmune or other inflammatory disease is systemic sclerosis.

15. A method for determining TKI therapy responsiveness of a patient afflicted with an autoimmune disease or other inflammatory disease comprising
determining in a biological sample of the patient the expression levels for a group of genes selected from the TKI Responsive Signature,
providing the expression levels to an entity for determining TKI responsiveness and selection of TKI therapy.

16. The method of claim 15, wherein the entity is a hospital, clinical center, or physician treating the patient.

17. An array comprising polynucleotides hybridizing to a group of genes selected from the TKI Responsive Signature.

18. A kit comprising primers or probes suitable for detecting the expression levels of a group of genes selected from the TKI Responsive Signature.

19. (canceled)

20. A method for identifying a TKI responsive gene expression profile comprising determining gene expression levels for a group of genes selected from the TKI Responsive Signature in a biological sample from a patient who is a candidate for TKI therapy.

21. The method of claim 20, wherein the group of genes are selected from the TKI Responsive Signature listed in Table 2, 3, 5, 6, 7, or 8.

22. The method of claim 20, wherein the patient has an autoimmune disease or another inflammatory disease.

23. The method of claim 1, wherein the group of genes is selected from TKI Responsive Signature listed in Table 7 and 8 and wherein the TKI therapy includes one or more inhibitors for PDGFR, Abl, and Kit.

24. The method of claim 1, wherein the collection of the mRNA expression levels within the predetermined responsive expression profile includes at least 50%, 60%, 70%, 80%, 90% or 95% of the genes within the group of genes selected from the TKI Responsive Signature having their expression levels within the predetermined responsive expression profile.

25. The method of claim 1 further comprising selecting or recommending a TKI therapy based on the expression levels for the group of genes selected from the TKI Responsive Signature in comparison to the TKI Responsive Signature.
